(19) United States
(12) Patent Application Publication
(10) Pub. No.: US 2025/0034109 A1
(43) Pub. Date: Jan. 30, 2025

(54) SALTS, COCRYSTALS, PHARMACEUTICAL COMPOSITIONS THEREOF, AND METHODS OF TREATMENT INVOLVING THE SAME

(71) Applicant: Servier Pharmaceuticals LLC, Boston, MA (US)

(72) Inventor: Julien Mahieux, Orléans (FR)

(21) Appl. No.: 18/783,908

(22) Filed: Jul. 25, 2024

(30) Foreign Application Priority Data
Jul. 25, 2023 (EP) 23306287.6

(51) Int. Cl. C07D 401/04; A61K 9/14; A61K 31/53

(52) U.S. Cl. CPC C07D 401/04; A61K 9/146; A61K 31/53

(57) ABSTRACT

Provided are solid forms of a compound useful for treating cancer, such as pharmaceutically acceptable salts and cocrystals, pharmaceutical compositions thereof, and use for the treatment cancer comprising administering the solid forms described herein to a patient in need thereof.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684 (1998); Geisbrecht et al., J. Biol. Chem. 274:30527-30533 (1999); Wiemann et al., Genome Res. 11:422-435 (2001); The MGC Project Team, Genome Res. 14:2121-2127 (2004); Lubec et al., Submitted (DEC-2008) to UniProtKB; Kullmann et al., Submitted (JUN-1996) to the EMBL/GenBank/DDBJ databases; and Sjoeblom et al., Science 314:268-274 (2006).

Non-mutant, e.g., wild type, IDH1 catalyzes the oxidative decarboxylation of isocitrate to α-ketoglutarate.

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of 2HG is believed to contribute to the formation and progression of cancer (Dang, L et al., Nature 2009, 462:739-44).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (NOV-1992) to the EMBL/GenBank/DDBJ databases; and The MGC Project Team, Genome Res. 14:2121-2127 (2004).

Non-mutant, e.g., wild type, IDH2 catalyzes the oxidative decarboxylation of isocitrate to α-ketoglutarate (α-KG).

It has been discovered that mutations of IDH2 present in certain cancer cells result in a new ability of the enzyme to catalyze the NADPH-dependent reduction of α-ketoglutarate to R(−)-2-hydroxyglutarate (2HG). 2HG is not formed by wild-type IDH2. The production of 2HG is believed to contribute to the formation and progression of cancer (Dang, L et al, Nature 2009, 462:739-44).

U.S. Publication No. 2015/0018328 A1 discloses a compound described by the chemical name 6-(6-chloropyridin-2-yl)-N²,N⁴-bis((R)-1,1,1-trifluoropropan-2-yl)-1,3,5-triazine-2,4-diamine, which has been shown to act as an inhibitor of mutant IDH1 and IDH2 proteins in biochemical and cellular assays.

### CROSS-REFERENCE TO RELATED APPLICATIONS

This application claims the right of priority based on EP application Ser. No. 23/306,287.6, filed Jul. 25, 2023, which is herein incorporated in its entirety by reference.

### SUMMARY

The present disclosure relates to solid forms (e.g., salts, cocrystals and other crystalline forms) of a compound of formula (I)

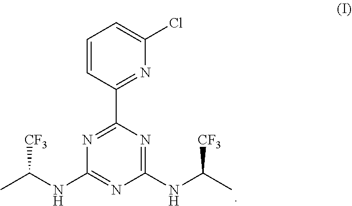 [SMILES: C[C@@H](Nc1nc(N[C@H](C)C(F)(F)F)nc(-c2cccc(Cl)n2)n1)C(F)(F)F]

In one aspect, the disclosure relates to solid forms of compound of formula (I).

In one aspect, the disclosure relates to pharmaceutically acceptable salts of the compound of formula (I).

In one aspect, the disclosure relates to crystalline forms of pharmaceutically acceptable salts of the compound of formula (I).

In one aspect, the disclosure relates to cocrystals comprising the compound of formula (I).

In one aspect, the present application relates to an amorphous solid dispersion comprising a compound of formula (I).

In one aspect, the present application relates to an amorphous solid dispersion prepared from a pharmaceutically acceptable salt of a compound of formula (I).

In one aspect, the present application relates to an amorphous solid dispersion prepared from a cocrystal comprising a compound of formula (I).

In another aspect, the present application relates to a pharmaceutical composition comprising a solid form of the compound of formula (I) and one or more pharmaceutical excipients.

In another aspect, the present application relates to a pharmaceutical composition comprising a pharmaceutically acceptable salt of the compound of formula (I) and one or more pharmaceutical excipients.

In another aspect, the present application relates to a pharmaceutical composition comprising a cocrystal of the compound of formula (I) and one or more pharmaceutical excipients.

In another aspect, the present application relates to a pharmaceutical composition comprising an amorphous solid dispersion comprising a solid form of the compound of formula (I), and one or more pharmaceutical excipients. In another aspect, the present application relates to a pharmaceutical composition comprising an amorphous solid dispersion comprising a pharmaceutically acceptable salt of the compound of formula (I), and one or more pharmaceutical excipients.

In another aspect, the present application relates to a pharmaceutical composition comprising an amorphous solid dispersion prepared from a cocrystal of the compound of formula (I), and one or more pharmaceutical excipients.

In another aspect, the present application relates to a method of preparation of a solid form of the compound of formula (I).

In another aspect, the present application relates to a method of preparation of a pharmaceutically acceptable salt of the compound of formula (I).

In another aspect, the present application relates to a method of preparation of a cocrystal of the compound of formula (I).

In another aspect, the present application relates to a method of treating a cancer characterized by the presence of an IDH1 and/or IDH2 mutation in a patient in need thereof, comprising administering a therapeutically effective amount of a solid form of the compound of formula (I), or a pharmaceutical composition thereof, to the patient.

In another aspect, the present application relates to a method of treating a cancer characterized by the presence of an IDH1 and/or IDH2 mutation in a patient in need thereof, comprising administering a therapeutically effective amount of a pharmaceutically acceptable salt of the compound of formula (I), or a pharmaceutical composition thereof, to the patient.

In another aspect, the present application relates to a method of treating a cancer characterized by the presence of an IDH1 and/or IDH2 mutation in a patient in need thereof, comprising administering a therapeutically effective amount of a cocrystal of the compound of formula (I), or a pharmaceutical composition thereof, to the patient.

### BRIEF DESCRIPTION OF THE DRAWINGS

FIG. 1 depicts the X-ray diffraction spectrum of the benzenesulfonic acid salt of compound of formula (I).

FIG. 2 depicts the ¹H NMR spectrum of the benzenesulfonic acid salt of compound of formula (I).

FIG. 3 depicts the X-ray diffraction spectrum of the (+)-camphor-10-sulfonic acid salt of compound of formula (I).

FIG. 4 depicts the ¹H NMR spectrum of the (+)-camphor-10-sulfonic acid salt of compound of formula (I).

FIG. 5 depicts the X-ray diffraction spectrum of the ethane-1,2-disulfonic acid salt of compound of formula (I).

FIG. 6 depicts the ¹H NMR spectrum of the ethane-1,2-disulfonic acid salt of compound of formula (I).

FIG. 7 depicts the X-ray diffraction spectrum of the ethanesulfonic acid salt of compound of formula (I).

FIG. 8 depicts the ¹H NMR spectrum of the ethanesulfonic acid salt of compound of formula (I).

FIG. 9 depicts the X-ray diffraction spectrum of the hydrobromic acid salt of compound of formula (I).

FIG. 10 depicts the ¹H NMR spectrum of the hydrobromic acid salt of compound of formula (I).

FIG. 11 depicts the X-ray diffraction spectrum of the hydrochloric acid salt of compound of formula (I).

FIG. 12 depicts the ¹H NMR spectrum of the hydrochloric acid salt of compound of formula (I).

FIG. 13 depicts the X-ray diffraction spectrum of the naphthalene-1,5-disulfonic acid salt of compound of formula (I).

FIG. 14 depicts the ¹H NMR spectrum of the naphthalene-1,5-disulfonic acid salt of compound of formula (I).

FIG. 15 depicts the X-ray diffraction spectrum of the naphthalene-2-sulfonic acid salt of compound of formula (I).

FIG. 16 depicts the ¹H NMR spectrum of the naphthalene-2-sulfonic acid salt of compound of formula (I).

FIG. 17 depicts the X-ray diffraction spectrum of the p-toluenesulfonic acid salt of compound of formula (I).

FIG. 18 depicts the ¹H NMR spectrum of the p-toluenesulfonic acid salt of compound of formula (I).

FIG. 19 depicts the X-ray diffraction spectrum of the sulfuric acid salt of compound of formula (I).

FIG. 20 depicts the ¹H NMR spectrum of the sulfuric acid salt of compound of formula (I).

FIG. 21 depicts the X-ray diffraction spectrum of the 3-hydroxy-2-naphtoic acid cocrystal of compound of formula (I).

FIG. 22 depicts the ¹H NMR spectrum of the 3-hydroxy-2-naphtoic acid cocrystal of compound of formula (I).

FIG. 23 depicts the X-ray diffraction spectrum of the L-serine cocrystal of compound of formula (I).

FIG. 24 depicts the ¹H NMR spectrum of the L-serine cocrystal of compound of formula (I).

FIG. 25 depicts the X-ray diffraction spectrum of the glycine cocrystal of compound of formula (I).

FIG. 26 depicts the ¹H NMR spectrum of the glycine cocrystal of compound of formula (I).

FIG. 27 depicts the X-ray diffraction spectrum of the D-gluconic acid cocrystal of compound of formula (I).

FIG. 28 depicts the ¹H NMR spectrum of the D-gluconic acid cocrystal of compound of formula (I).

FIG. 29 depicts Differential Scanning calorimetry (DSC) thermogram of the ethane-1,2-disulfonic acid salt of compound of formula (I).

FIG. 30 depicts DSC thermogram of the naphthalene-1,5-disulfonic acid salt of compound of formula (I).

FIG. 31 depicts DSC thermogram of the 3-hydroxy-2-naphtoic acid cocrystal of compound of formula (I).

FIG. 32 depicts the ¹H NMR spectrum of the glycolic acid cocrystal of compound of formula (I).

FIG. 33 depicts the X-ray diffraction spectrum of the glycolic acid cocrystal of compound of formula (I).

FIG. 34 depicts the ¹H NMR spectrum of the L-malic acid cocrystal of compound of formula (I).

FIG. 35 depicts the X-ray diffraction spectrum of the L-malic acid cocrystal of compound of formula (I).

FIG. 36 depicts the ¹H NMR spectrum of the oxalic acid cocrystal of compound of formula (I).

FIG. 37 depicts the X-ray diffraction spectrum of the oxalic acid cocrystal of compound of formula (I).

FIG. 38 depicts the ¹H NMR spectrum of the benzoic acid cocrystal of compound of formula (I).

FIG. 39 depicts the X-ray diffraction spectrum of the benzoic acid cocrystal of compound of formula (I).

FIG. 40 depicts DSC thermogram of the benzoic acid cocrystal of compound of formula (I).

FIG. 41 depicts the ¹H NMR spectrum of the fumaric acid cocrystal of compound of formula (I).

FIG. 42 depicts the X-ray diffraction spectrum of the fumaric acid cocrystal of compound of formula (I).

FIG. 43 depicts the ¹H NMR spectrum of the gentisic acid cocrystal of compound of formula (I).

FIG. 44 depicts the X-ray diffraction spectrum of the gentisic acid cocrystal of compound of formula (I).

FIG. 45 depicts the ¹H NMR spectrum of the glutaric acid cocrystal of compound of formula (I).

FIG. 46 depicts the X-ray diffraction spectrum of the glutaric acid cocrystal of compound of formula (I).

FIG. 47 depicts the ¹H NMR spectrum of the ortho phosphoric acid salt of compound of formula (I).

FIG. 48 depicts the X-ray diffraction spectrum of the ortho phosphoric acid salt of compound of formula (I).

FIG. 49 depicts the ¹H NMR spectrum of the 4-hydroxybenzoic acid cocrystal of compound of formula (I).

FIG. 50 depicts the X-ray diffraction spectrum of the 4-hydroxybenzoic acid cocrystal of compound of formula (I).

FIG. 51 depicts the ¹H NMR spectrum of the alpha-ketoglutaric acid cocrystal of compound of formula (I).

FIG. 52 depicts the X-ray diffraction spectrum of the alpha-ketoglutaric acid cocrystal of compound of formula (I).

FIG. 53 depicts the ¹H NMR spectrum of the malonic acid cocrystal of compound of formula (I).

FIG. 54 depicts the X-ray diffraction spectrum of the malonic acid cocrystal of compound of formula (I).

FIG. 55 depicts the ¹H NMR spectrum of the salicylic acid cocrystal of compound of formula (I).

FIG. 56 depicts the X-ray diffraction spectrum of the salicylic acid cocrystal of compound of formula (I).

FIG. 57 depicts the ¹H NMR spectrum of the L-tartaric acid cocrystal of compound of formula (I).

FIG. 58 depicts the X-ray diffraction spectrum of the L-tartaric acid cocrystal of compound of formula (I).

FIG. 59 depicts the ¹H NMR spectrum of the urea cocrystal of compound of formula (I).

FIG. 60 depicts the X-ray diffraction spectrum of the urea cocrystal of compound of formula (I).

FIG. 61 depicts the ¹H NMR spectrum of the pyroglutamic acid cocrystal of compound of formula (I).

FIG. 62 depicts the X-ray diffraction spectrum of the pyroglutamic acid cocrystal of compound of formula (I).

FIG. 63 depicts the ¹H NMR spectrum of the caproic acid cocrystal of compound of formula (I).

FIG. 64 depicts the X-ray diffraction spectrum of the caproic acid cocrystal of compound of formula (I).

FIG. 65 depicts the ¹H NMR spectrum of the glycerol cocrystal of compound of formula (I).

FIG. 66 depicts the X-ray diffraction spectrum of the glycerol cocrystal of compound of formula (I).

FIG. 67 depicts the ¹H NMR spectrum of the L-lysine cocrystal of compound of formula (I).

FIG. 68 depicts the X-ray diffraction spectrum of the L-lysine cocrystal of compound of formula (I).

FIG. 69 depicts the ¹H NMR spectrum of the S-proline cocrystal of compound of formula (I).

FIG. 70 depicts the X-ray diffraction spectrum of the S-proline cocrystal of compound of formula (I).

FIG. 71 depicts the ¹H NMR spectrum of the pyruvic acid cocrystal of compound of formula (I).

FIG. 72 depicts the X-ray diffraction spectrum of the pyruvic acid cocrystal of compound of formula (I).

### DETAILED DESCRIPTION

The present disclosure relates to solid forms of a compound of formula (I), as defined herein, pharmaceutical compositions comprising same, methods of preparing same, and methods of treatment involving same.

A synthesis of compound of formula (I), that is 6-(6-chloropyridin-2-yl)-N²,N⁴-bis((R)-1,1,1-trifluoropropan-2-yl)-1,3,5-triazine-2,4-diamine, is described in paragraphs [1032]-[1036] of U.S. Publication No. 2015/0018328 A1.

An alternate method for the synthesis of compound of formula (I) is disclosed in paragraphs [0343]-[0349] of US Publication No. 2021/0198234.

Under some conditions, compound of formula (I) exists at least in part in one or more tautomeric forms, including without limitation one or more of the following:

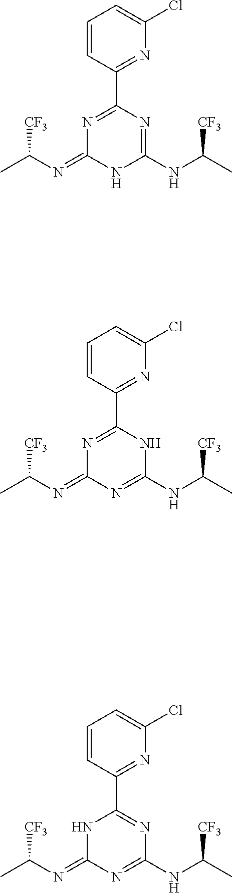

As used herein, the term compound of formula (I) shall be understood to refer to 6-(6-chloropyridin-2-yl)-N²,N⁴-bis((R)-1,1,1-trifluoropropan-2-yl)-1,3,5-triazine-2,4-diamine or any tautomer(s) thereof. The double bond geometries of the foregoing tautomers were not determined, and therefore the chemical structures representing the foregoing tautomers are not intended to imply a particular double bond geometry.

The existence of one or more tautomers of compound of formula (I) was investigated in paragraphs [0321]-[0325] of US Publication No 2021/0198234.

As used herein, the compound of formula (I) includes the compound having the identified chemical structure, as well as any rotamer thereof.

In the specification and claims, each atom of the compound of formula (I) is meant to represent any stable isotope of the specified element. In the Examples, no effort was made to enrich any atom of compound of formula (I) in a particular isotope, and therefore each atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

In some embodiments, the compound of formula (I) includes each constituent atom at approximately the natural abundance isotopic composition of the specified element.

In one aspect, the disclosure relates to solid forms of a compound of formula (I)

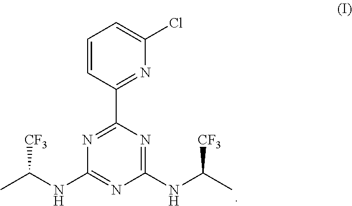 [SMILES: C[C@@H](Nc1nc(N[C@H](C)C(F)(F)F)nc(-c2cccc(Cl)n2)n1)C(F)(F)F]

In one aspect, the disclosure relates to pharmaceutically acceptable salts of the compound of formula (I).

In another aspect, the disclosure relates to crystalline forms of pharmaceutically acceptable salts of the compound of formula (I).

In one aspect, the pharmaceutically acceptable salts of the compound of formula (I) are formed using a pharmaceutically acceptable acid selected from the group consisting of benzene sulfonic acid, (+)-camphor-10-sulfonic acid, ethane-1,2-disulfonic acid, ethanesulfonic acid, hydrobromic acid, hydrochloric acid, naphthalene-1,5-disulfonic acid, naphthalene-2-sulfonic acid, p-toluenesulfonic acid sulfuric acid or ortho phosphoric acid.

In one aspect, the disclosure relates to cocrystals comprising the compound of formula (I).

In one aspect, the disclosure relates to cocrystals comprising the compound of formula (I) and a coformer selected the group consisting of from 3-hydroxy-2-naphtoic acid, L-serine, glycine, D-gluconic acid, glycolic acid, L-malic acid, oxalic acid, benzoic acid, fumaric acid, gentisic acid, glutaric acid, 4-hydroxybenzoic acid, alpha-ketoglutaric acid, malonic acid, salicylic acid, L-tartaric acid, urea, pyroglutamic acid caproic acid, glycerol, L-lysine, S-proline and pyruvic acid.

Pyroglutamic acid is used herein to designate 2-pyrolidone-5-carboxylic acid,

As used herein, the term “cocrystal” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species.

The species making up a cocrystal typically are linked by hydrogen bonding and other non-covalent and non-ionic interactions. Thus, a pharmaceutical cocrystal of a drug typically comprises the drug and one or more coformers. The combinations of drug and coformer(s) that will form cocrystals generally cannot be predicted ab initio, and cocrystal formation typically affects the physicochemical properties of a drug in unpredictable ways.

As used herein, the term “crystalline” refers to a solid material whose constituent particles (e.g., molecules) are arranged spatially in a regular and repeating lattice.

In one aspect, the pharmaceutically acceptable salt of the compound of formula (I) is a benzenesulfonic acid salt.

In another aspect, the benzenesulfonic acid salt of the compound of formula (I) is crystalline.

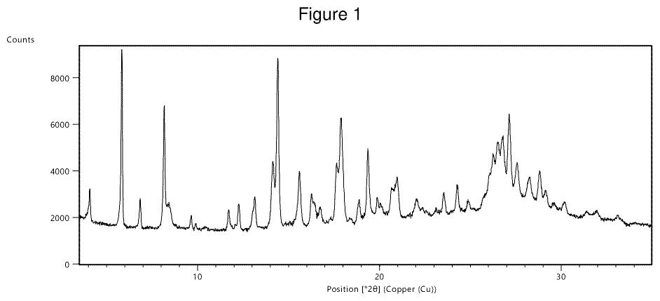

In some embodiments, the crystalline benzenesulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 3 below. In other embodiments, the X-ray powder diffraction pattern comprises at least one peak position, in degrees 2-theta (±0.2 degrees 2-theta), selected from the group consisting of 5.83, 8.17, 14.40 and 17.90. In other embodiments, the X-ray powder diffraction pattern comprises at least two peak positions, in degrees 2-theta (±0.2 degrees 2-theta), selected from the group consisting of 5.83, 8.17, 14.40 and 17.90. In other embodiments, the X-ray powder diffraction pattern comprises at least three peak positions, in degrees 2-theta (±0.2 degrees 2-theta), selected from the group consisting of 5.83, 8.17, 14.40 and 17.90. In other embodiments, the X-ray powder diffraction pattern comprises the four peak positions, in degrees 2-theta (±0.2 degrees 2-theta) 5.83, 8.17, 14.40 and 17.90. In other embodiments, the X-ray powder diffraction pattern comprises peak positions, in degrees 2-theta (±0.2 degrees 2-theta), of 5.83 and 14.40 and, and at least one peak position selected from the group consisting of 8.17 and 17.90. In other embodiments, the X-ray powder diffraction pattern comprises the peak positions, in degrees 2-theta (±0.2 degrees 2-theta), set forth in Table 3. In other embodiments, the X-ray powder diffraction pattern is similar to the X-ray powder diffraction pattern shown in FIG. 1.

As used herein, where an X-ray powder diffraction pattern is described as having a specified number of peak positions, “in degrees 2-theta (±0.2 degrees 2-theta),” selected from a specified group of peak positions, the margin of error (±0.2 degrees 2-theta) shall be understood to apply to each peak position within the group.

As used herein, the term “similar,” when referring to two or more X-ray powder diffraction patterns, means that the patterns would be understood by a person of ordinary skill in the art to represent the same crystalline form and that the patterns are the same, except for the types of variations that would be expected by a person of ordinary skill in the art to arise from experimental variations, such as instrumentation used, time of day, humidity, season, pressure, temperature, etc.

In one aspect, the pharmaceutically acceptable salt of the compound of formula (I) is a (+)-camphor-10-sulfonic acid salt.

In another aspect, the (+)-camphor-10-sulfonic acid salt of the compound of formula (I) is crystalline.

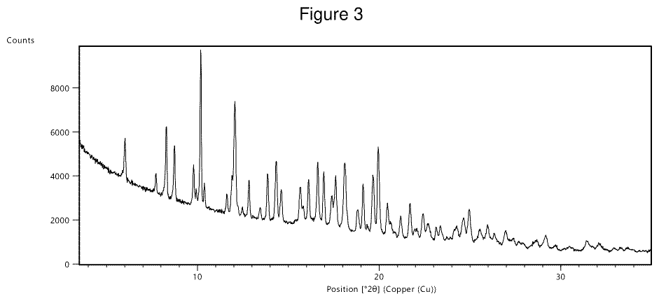

In some embodiments, the crystalline (+)-camphor-10-sulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 4 below. In other embodiments, the X-ray powder diffraction pattern comprises at least one peak position, in degrees 2-theta (±0.2 degrees 2-theta), selected from the group consisting of 8.29, 10.19, 12.07, 18.12 and 19.97. In other embodiments, the X-ray powder diffraction pattern comprises at least two peak positions, in degrees 2-theta (±0.2 degrees 2-theta), selected from the group consisting of 8.29, 10.19, 12.07, 18.12 and 19.97. In other embodiments, the X-ray powder diffraction pattern comprises at least three peak positions, in degrees 2-theta (±0.2 degrees 2-theta), selected from the group consisting of 8.29, 10.19, 12.07, 18.12 and 19.97. In other embodiments, the X-ray powder diffraction pattern comprises at least four peak positions, in degrees 2-theta (±0.2 degrees 2-theta), selected from the group consisting of 8.29, 10.19, 12.07, 18.12 and 19.97. In other embodiments, the X-ray powder diffraction pattern comprises peak positions, in degrees 2-theta (±0.2 degrees 2-theta), of 10.19 and 12.07, and at least one peak position selected from the group consisting of 8.29, 18.12 and 19.97. In other embodiments, the X-ray powder diffraction pattern comprises the peak positions, in degrees 2-theta (±0.2 degrees 2-theta), set forth in Table 4. In other embodiments, the X-ray powder diffraction pattern is similar to the X-ray powder diffraction pattern shown in FIG. 3.

In one aspect, the pharmaceutically acceptable salt of the compound of formula (I) is an ethane-1,2-disulfonic acid salt.

In another aspect, the ethane-1,2-disulfonic acid salt of the compound of formula (I) is crystalline.

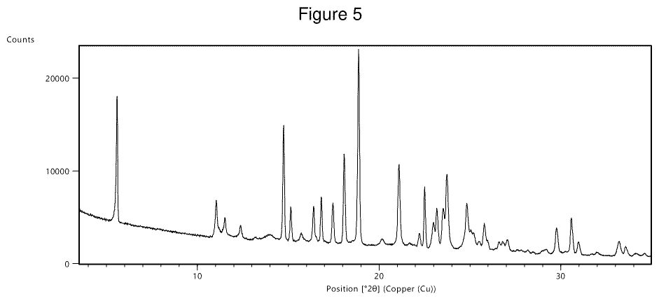

In some embodiments, the crystalline ethane-1,2-disulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 5 below. In other embodiments, the X-ray powder diffraction pattern comprises at least one peak position, in degrees 2-theta (±0.2 degrees 2-theta), selected from the group consisting of 5.59, 14.75, 18.08 and 18.87. In other embodiments, the X-ray powder diffraction pattern comprises at least two peak positions, in degrees 2-theta (±0.2 degrees 2-theta), selected from the group consisting of 5.59, 14.75, 18.08 and 18.87. In other embodiments, the X-ray powder diffraction pattern comprises at least three peak positions, in degrees 2-theta (±0.2 degrees 2-theta), selected from the group consisting of 5.59, 14.75, 18.08 and 18.87. In other embodiments, the X-ray powder diffraction pattern comprises four peak positions 5.59, 14.75, 18.08 and 18.87. In other embodiments, the X-ray powder diffraction pattern comprises peak positions, in degrees 2-theta (±0.2 degrees 2-theta), of 5.59 and 18.87, and at least one peak position selected from the group consisting of 14.75 and 18.08. In other embodiments, the X-ray powder diffraction pattern comprises the peak positions, in degrees 2-theta (±0.2 degrees 2-theta), set forth in Table 5. In other embodiments, the X-ray powder diffraction pattern is similar to the X-ray powder diffraction pattern shown in FIG. 5.

In some embodiments, the ethane-1,2-disulfonic acid salt of the compound of formula (I) is characterized by a differential scanning calorimetry thermogram comprising an endothermic peak having an onset temperature of 304.30° C. (+5.0° C.). In other embodiments, the ethane-1,2-disulfonic acid salt of the compound of formula (I) is characterized by a differential scanning calorimetry thermogram comprising an endothermic peak having an onset temperature of 304.30° C. (±2.0° C.).

In one aspect, the pharmaceutically acceptable salt of the compound of formula (I) is an ethanesulfonic acid salt.

In another aspect, the ethanesulfonic acid salt of the compound of formula (I) is crystalline.

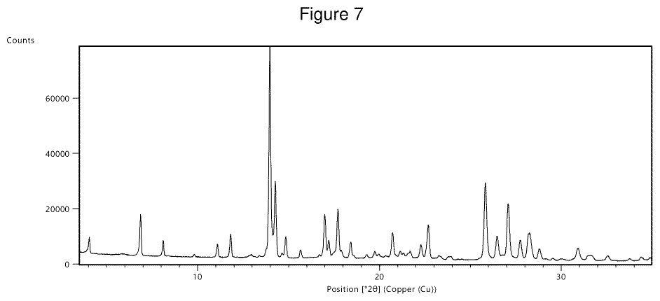

In some embodiments, the crystalline ethanesulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 6 below. In other embodiments, the X-ray powder diffraction pattern comprises at least one peak position, in degrees 2-theta (±0.2 degrees 2-theta), selected from the group consisting of 6.86, 13.97, 14.27, 17.00 and 17.72. In other embodiments, the X-ray powder diffraction pattern comprises at least two peak positions, in degrees 2-theta (±0.2 degrees 2-theta), selected from the group consisting of 6.86, 13.97, 14.27, 17.00 and 17.72. In other embodiments, the X-ray powder diffraction pattern comprises at least three peak positions, in degrees 2-theta (±0.2 degrees 2-theta), selected from the group consisting of 6.86, 13.97, 14.27, 17.00 and 17.72. In other embodiments, the X-ray powder diffraction pattern comprises at least four peak positions, in degrees 2-theta (±0.2 degrees 2-theta), selected from the group consisting of 6.86, 13.97, 14.27, 17.00 and 17.72. In other embodiments, the X-ray powder diffraction pattern comprises peak positions, in degrees 2-theta (±0.2 degrees 2-theta), of 13.97 and 14.27, and at least one peak position selected from the group consisting of 6.86, 17.00 and 17.72. In other embodiments, the X-ray powder diffraction pattern comprises the peak positions, in degrees 2-theta (±0.2 degrees 2-theta), set forth in Table 6. In other embodiments, the X-ray powder diffraction pattern is similar to the X-ray powder diffraction pattern shown in FIG. 7.

In one aspect, the pharmaceutically acceptable salt of the compound of formula (I) is a hydrobromic acid salt.

In another aspect, the hydrobromic acid salt of the compound of formula (I) is crystalline.

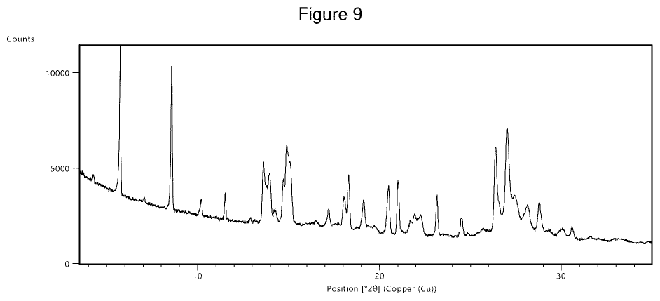

In some embodiments, the crystalline hydrobromic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 7 below. In other embodiments, the X-ray powder diffraction pattern comprises at least one peak position, in degrees 2-theta (±0.2 degrees 2-theta), selected from the group consisting of 5.74, 8.56, 13.63, 14.90 and 15.08. In other embodiments, the X-ray powder diffraction pattern comprises at least two peak positions, in degrees 2-theta (±0.2 degrees 2-theta), selected from the group consisting of 5.74, 8.56, 13.63, 14.90 and 15.08. In other embodiments, the X-ray powder diffraction pattern comprises at least three peak positions, in degrees 2-theta (±0.2 degrees 2-theta), selected from the group consisting of 5.74, 8.56, 13.63, 14.90 and 15.08. In other embodiments, the X-ray powder diffraction pattern comprises at least four peak positions, in degrees 2-theta (±0.2 degrees 2-theta), selected from the group consisting of 5.74, 8.56, 13.63, 14.90 and 15.08. In other embodiments, the X-ray powder diffraction pattern comprises peak positions, in degrees 2-theta (±0.2 degrees 2-theta), of 5.74 and 8.56, and at least one peak position selected from the group consisting of 13.63, 14.90 and 15.08. In other embodiments, the X-ray powder diffraction pattern comprises the peak positions, in degrees 2-theta (±0.2 degrees 2-theta), set forth in Table 7. In other embodiments, the X-ray powder diffraction pattern is similar to the X-ray powder diffraction pattern shown in FIG. 9.

In one aspect, the pharmaceutically acceptable salt of the compound of formula (I) is a hydrochloric acid salt.

In another aspect, the hydrochloric acid salt of the compound of formula (I) is crystalline.

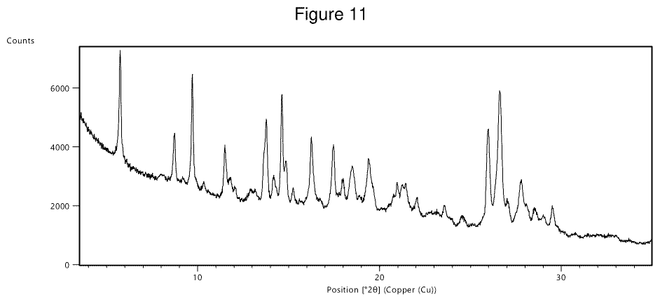

In some embodiments, the crystalline hydrochloric acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 8 below. In other embodiments, the X-ray powder diffraction pattern comprises at least one peak position, in degrees 2-theta (±0.2 degrees 2-theta), selected from the group consisting of 5.73, 9.71, 13.78, 14.64 and 16.25. In other embodiments, the X-ray powder diffraction pattern comprises at least two peak positions, in degrees 2-theta (±0.2 degrees 2-theta), selected from the group consisting of 5.73, 9.71, 13.78, 14.64 and 16.25. In other embodiments, the X-ray powder diffraction pattern comprises at least three peak positions, in degrees 2-theta (±0.2 degrees 2-theta), selected from the group consisting of 5.73, 9.71, 13.78, 14.64 and 16.25. In other embodiments, the X-ray powder diffraction pattern comprises at least four peak positions, in degrees 2-theta (±0.2 degrees 2-theta), selected from the group consisting of 5.73, 9.71, 13.78, 14.64 and 16.25. In other embodiments, the X-ray powder diffraction pattern comprises peak positions, in degrees 2-theta (±0.2 degrees 2-theta), of 5.73 and 14.64, and at least one peak position selected from the group consisting of 9.71, 13.78 and 16.25. In other embodiments, the X-ray powder diffraction pattern comprises the peak positions, in degrees 2-theta (±0.2 degrees 2-theta), set forth in Table 8. In other embodiments, the X-ray powder diffraction pattern is similar to the X-ray powder diffraction pattern shown in FIG. 11.

In one aspect, the pharmaceutically acceptable salt of the compound of formula (I) is a naphthalene-1,5-disulfonic acid salt.

In another aspect, the naphthalene-1,5-disulfonic acid salt of the compound of formula (I) is crystalline.

In some embodiments, the crystalline naphthalene-1,5-disulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 9 below. In other embodiments, the X-ray powder diffraction pattern comprises at least one peak position, in degrees 2-theta (±0.2 degrees 2-theta), selected from the group consisting of 5.52, 13.79, 14.17, 19.28, 19.63 and 19.97. In other embodiments, the X-ray powder diffraction pattern comprises at least two peak positions, in degrees 2-theta (±0.2 degrees 2-theta), selected from the group consisting of 5.52, 13.79, 14.17, 19.28, 19.63 and 19.97.

In other embodiments, the X-ray powder diffraction pattern comprises at least three peak positions, in degrees 2-theta (±0.2 degrees 2-theta), selected from the group consisting of 5.52, 13.79, 14.17, 19.28, 19.63 and 19.97. In other embodiments, the X-ray powder diffraction pattern comprises at least four peak positions, in degrees 2-theta (±0.2 degrees 2-theta), selected from the group consisting of 5.52, 13.79, 14.17, 19.28, 19.63 and 19.97.

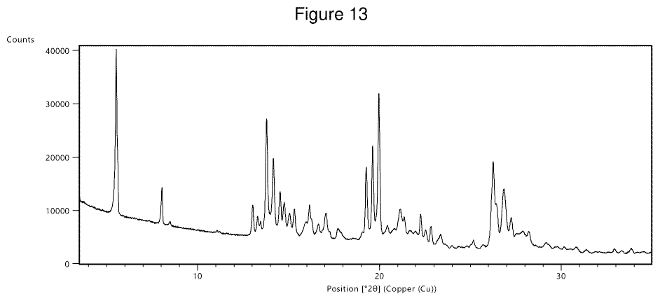

In other embodiments, the X-ray powder diffraction pattern comprises peak positions, in degrees 2-theta (±0.2 degrees 2-theta), of 5.52 and 19.97, and at least one peak position selected from the group consisting of 13.79, 14.17, 19.28 and 19.63. In other embodiments, the X-ray powder diffraction pattern comprises the peak positions, in degrees 2-theta (±0.2 degrees 2-theta), set forth in Table 9. In other embodiments, the X-ray powder diffraction pattern is similar to the X-ray powder diffraction pattern shown in FIG. 13.

In some embodiments, the naphthalene-1,5-disulfonic acid salt of the compound of formula (I) is characterized by a differential scanning calorimetry thermogram comprising an endothermic peak having an onset temperature of 324.30° C. (+5.0° C.). In other embodiments, the naphthalene-1,5-disulfonic acid salt of the compound of formula (I) is characterized by a differential scanning calorimetry thermogram comprising an endothermic peak having an onset temperature of 324.30° C. (±2.0° C.).

In one aspect, the pharmaceutically acceptable salt of the compound of formula (I) is a naphthalene-2-sulfonic acid salt.

In another aspect, the naphthalene-2-sulfonic acid salt of the compound of formula (I) is crystalline.

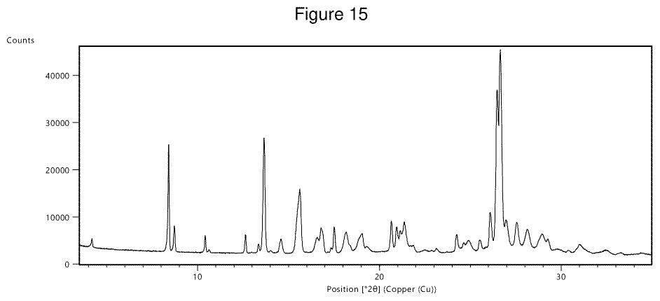

In some embodiments, the crystalline naphthalene-2-sulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 10 below. In other embodiments, the X-ray powder diffraction pattern comprises at least one peak position, in degrees 2-theta (±0.2 degrees 2-theta), selected from the group consisting of 8.41, 13.65, 26.09 and 26.47. In other embodiments, the X-ray powder diffraction pattern comprises at least two peak positions, in degrees 2-theta (±0.2 degrees 2-theta), selected from the group consisting of 8.41, 13.65, 26.09 and 26.47. In other embodiments, the X-ray powder diffraction pattern comprises at least three peak positions, in degrees 2-theta (±0.2 degrees 2-theta), selected from the group consisting of 8.41, 13.65, 26.09 and 26.47. In other embodiments, the X-ray powder diffraction pattern comprises the following four peak positions, in degrees 2-theta (±0.2 degrees 2-theta) selected from the group consisting of 8.41, 13.65, 26.09 and 26.47. In other embodiments, the X-ray powder diffraction pattern comprises peak positions, in degrees 2-theta (±0.2 degrees 2-theta), of 8.41 and 26.47, and at least one peak position selected from the group consisting of 13.65 and 26.09. In other embodiments, the X-ray powder diffraction pattern comprises the peak positions, in degrees 2-theta (±0.2 degrees 2-theta), set forth in Table 10. In other embodiments, the X-ray powder diffraction pattern is similar to the X-ray powder diffraction pattern shown in FIG. 15.

In one aspect, the pharmaceutically acceptable salt of the compound of formula (I) is a p-toluenesulfonic acid salt.

In another aspect, the p-toluenesulfonic acid salt of the compound of formula (I) is crystalline.

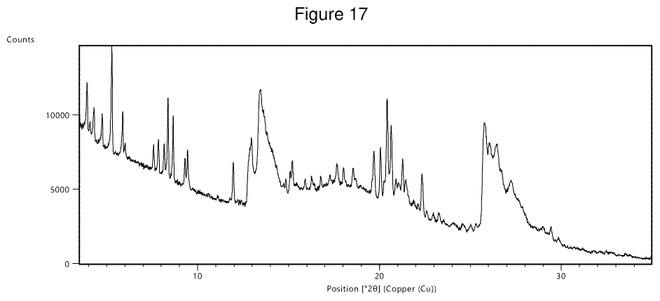

In some embodiments, the crystalline p-toluenesulfonic acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 11 below. In other embodiments, the X-ray powder diffraction pattern comprises at least one peak position, in degrees 2-theta (±0.2 degrees 2-theta), selected from the group consisting of 5.29, 8.37, 8.66, 12.96, 13.47, 20.42 and 20.64. In other embodiments, the X-ray powder diffraction pattern comprises at least two peak positions, in degrees 2-theta (±0.2 degrees 2-theta), selected from the group consisting of 5.29, 8.37, 8.66, 12.96, 13.47, 20.42 and 20.64. In other embodiments, the X-ray powder diffraction pattern comprises at least three peak positions, in degrees 2-theta (±0.2 degrees 2-theta), selected from the group consisting of 5.29, 8.37, 8.66, 12.96, 13.47, 20.42 and 20.64. In other embodiments, the X-ray powder diffraction pattern comprises at least four peak positions, in degrees 2-theta (±0.2 degrees 2-theta), selected from the group consisting of 5.29, 8.37, 8.66, 12.96, 13.47, 20.42 and 20.64. In other embodiments, the X-ray powder diffraction pattern comprises peak positions, in degrees 2-theta (±0.2 degrees 2-theta), of 5.29 and 12.96, and at least one peak position selected from the group consisting of 8.37, 8.66, 13.47, 20.42 and 20.64. In other embodiments, the X-ray powder diffraction pattern comprises the peak positions, in degrees 2-theta (±0.2 degrees 2-theta), set forth in Table 11. In other embodiments, the X-ray powder diffraction pattern is similar to the X-ray powder diffraction pattern shown in FIG. 17.

In one aspect, the pharmaceutically acceptable salt of the compound of formula (I) is a sulfuric acid salt.

In another aspect, the sulfuric acid salt of the compound of formula (I) is crystalline.

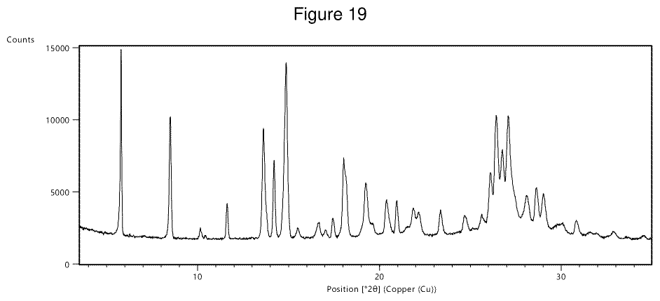

In some embodiments, the crystalline sulfuric acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 12 below. In other embodiments, the X-ray powder diffraction pattern comprises at least one peak position, in degrees 2-theta (±0.2 degrees 2-theta), selected from the group consisting of 5.80, 8.50, 13.62, 14.21, 14.87, and 18.04. In other embodiments, the X-ray powder diffraction pattern comprises at least two peak positions, in degrees 2-theta (±0.2 degrees 2-theta), selected from the group consisting of 5.80, 8.50, 13.62, 14.21, 14.87, and 18.04. In other embodiments, the X-ray powder diffraction pattern comprises at least three peak positions, in degrees 2-theta (±0.2 degrees 2-theta), selected from the group consisting of 5.80, 8.50, 13.62, 14.21, 14.87, and 18.04. In other embodiments, the X-ray powder diffraction pattern comprises at least four peak positions, in degrees 2-theta (±0.2 degrees 2-theta), selected from the group consisting of 5.80, 8.50, 13.62, 14.21, 14.87, and 18.04. In other embodiments, the X-ray powder diffraction pattern comprises peak positions, in degrees 2-theta (±0.2 degrees 2-theta), of 5.80 and 14.87, and at least one peak position selected from the group consisting of 8.50, 13.62, 14.21 and 18.04. In other embodiments, the X-ray powder diffraction pattern comprises the peak positions, in degrees 2-theta (±0.2 degrees 2-theta), set forth in Table 12. In other embodiments, the X-ray powder diffraction pattern is similar to the X-ray powder diffraction pattern shown in FIG. 19.

In one aspect, the pharmaceutically acceptable salt of the compound of formula (I) is an ortho phosphoric acid salt.

In another aspect, the ortho phosphoric acid salt of the compound of formula (I) is crystalline.

In some embodiments, the crystalline ortho phosphoric acid salt of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 24 below. In other embodiments, the X-ray powder diffraction pattern comprises at least one peak position, in degrees 2-theta (±0.2 degrees 2-theta), selected from the group consisting of 5.47, 14.27, 14.91, 18.35, 19.72 and 21.29. In other embodiments, the X-ray powder diffraction pattern comprises at least two peak positions, in degrees 2-theta (±0.2 degrees 2-theta), selected from the group consisting of 5.47, 14.27, 14.91, 18.35, 19.72 and 21.29. In other embodiments, the X-ray powder diffraction pattern comprises at least three peak positions, in degrees 2-theta (±0.2 degrees 2-theta), selected from the group consisting of 5.47, 14.27, 14.91, 18.35, 19.72 and 21.29. In other embodiments, the X-ray powder diffraction pattern comprises at least four peak positions, in degrees 2-theta (±0.2 degrees 2-theta), selected from the group consisting of 5.47, 14.27, 14.91, 18.35, 19.72 and 21.29.

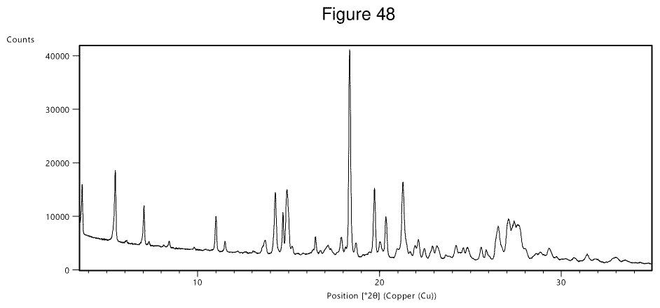

In other embodiments, the X-ray powder diffraction pattern comprises peak positions, in degrees 2-theta (±0.2 degrees 2-theta), of 18.35 and 21.29, and at least one peak position selected from the group consisting of 5.47, 14.27, 14.91 and 19.72. In other embodiments, the X-ray powder diffraction pattern comprises the peak positions, in degrees 2-theta (±0.2 degrees 2-theta), set forth in Table 24. In other embodiments, the X-ray powder diffraction pattern is similar to the X-ray powder diffraction pattern shown in FIG. 48.

In one aspect, the cocrystal comprising the compound of formula (I) is a 3-hydroxy-2-naphtoic acid cocrystal.

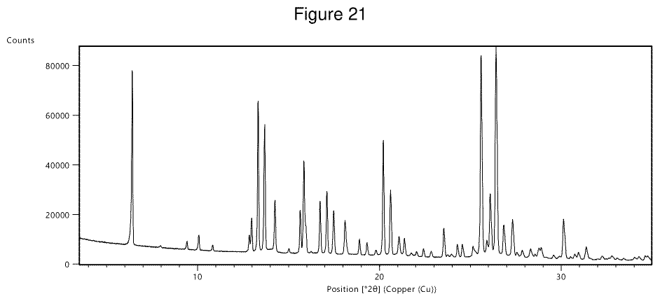

In some embodiments, the 3-hydroxy-2-naphtoic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 13 below. In other embodiments, the X-ray powder diffraction pattern comprises at least one peak position, in degrees 2-theta (±0.2 degrees 2-theta), selected from the group consisting of 6.41, 13.34, 13.69, 15.84 and 20.21. In other embodiments, the X-ray powder diffraction pattern comprises at least two peak positions, in degrees 2-theta (±0.2 degrees 2-theta), selected from the group consisting of 6.41, 13.34, 13.69, 15.84 and 20.21. In other embodiments, the X-ray powder diffraction pattern comprises at least three peak positions, in degrees 2-theta (±0.2 degrees 2-theta), selected from the group consisting of 6.41, 13.34, 13.69, 15.84 and 20.21. In other embodiments, the X-ray powder diffraction pattern comprises at least four peak positions, in degrees 2-theta (±0.2 degrees 2-theta), selected from the group consisting of 6.41, 13.34, 13.69, 15.84 and 20.21. In other embodiments, the X-ray powder diffraction pattern comprises peak positions, in degrees 2-theta (±0.2 degrees 2-theta), of 6.41 and 13.34, and at least one peak position selected from the group consisting of 13.69, 15.84 and 20.21. In other embodiments, the X-ray powder diffraction pattern comprises peak positions, in degrees 2-theta (±0.2 degrees 2-theta), of. In other embodiments, the X-ray powder diffraction pattern comprises the peak positions, in degrees 2-theta (±0.2 degrees 2-theta), set forth in Table 13. In other embodiments, the X-ray powder diffraction pattern is similar to the X-ray powder diffraction pattern shown in FIG. 21.

In some embodiments, the 3-hydroxy-2-naphtoic acid cocrystal of the compound of formula (I) is characterized by a differential scanning calorimetry thermogram comprising an endothermic peak having an onset temperature of 202.79° C. (+5.0° C.). In other embodiments, the 3-hydroxy-2-naphtoic acid cocrystal of the compound of formula (I) is characterized by a differential scanning calorimetry thermogram comprising an endothermic peak having an onset temperature of 202.79° C. (±2.0° C.).

In one aspect, the cocrystal comprising the compound of formula (I) is a L-serine cocrystal.

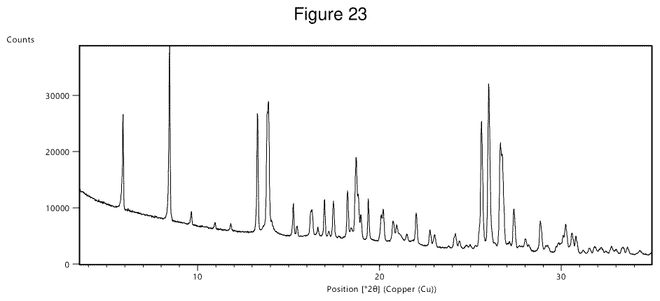

In some embodiments, the L-serine cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 14 below. In other embodiments, the X-ray powder diffraction pattern comprises at least one peak position, in degrees 2-theta (±0.2 degrees 2-theta), selected from the group consisting of 5.90, 8.46, 13.28, 13.88 and 18.72. In other embodiments, the X-ray powder diffraction pattern comprises at least two peak positions, in degrees 2-theta (±0.2 degrees 2-theta), selected from the group consisting of 5.90, 8.46, 13.28, 13.88 and 18.72. In other embodiments, the X-ray powder diffraction pattern comprises at least three peak positions, in degrees 2-theta (±0.2 degrees 2-theta), selected from the group consisting of 5.90, 8.46, 13.28, 13.88 and 18.72. In other embodiments, the X-ray powder diffraction pattern comprises at least four peak positions, in degrees 2-theta (±0.2 degrees 2-theta), selected from the group consisting of 5.90, 8.46, 13.28, 13.88 and 18.72. In other embodiments, the X-ray powder diffraction pattern comprises peak positions, in degrees 2-theta (±0.2 degrees 2-theta), of 8.46 and 13.88, and at least one peak position selected from the group consisting of 5.90, 13.28 and 18.72. In other embodiments, the X-ray powder diffraction pattern comprises peak positions, in degrees 2-theta (±0.2 degrees 2-theta), of. In other embodiments, the X-ray powder diffraction pattern comprises the peak positions, in degrees 2-theta (±0.2 degrees 2-theta), set forth in Table 14. In other embodiments, the X-ray powder diffraction pattern is similar to the X-ray powder diffraction pattern shown in FIG. 23.

In one aspect, the cocrystal comprising the compound of formula (I) is a glycine cocrystal.

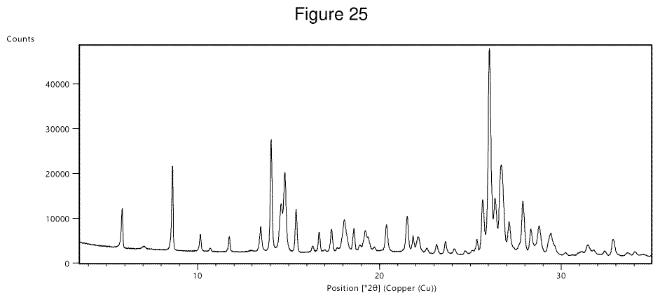

In some embodiments, the glycine cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 15 below. In other embodiments, the X-ray powder diffraction pattern comprises at least one peak position, in degrees 2-theta (±0.2 degrees 2-theta), selected from the group consisting of 8.62, 14.04, 14.79 and 26.04. In other embodiments, the X-ray powder diffraction pattern comprises at least two peak positions, in degrees 2-theta (±0.2 degrees 2-theta), selected from the group consisting of 8.62, 14.04, 14.79 and 26.04. In other embodiments, the X-ray powder diffraction pattern comprises at least three peak positions, in degrees 2-theta (±0.2 degrees 2-theta), selected from the group consisting of 8.62, 14.04, 14.79 and 26.04. In other embodiments, the X-ray powder diffraction pattern comprises the four following peak positions, in degrees 2-theta (±0.2 degrees 2-theta): 8.62, 14.04, 14.79 and 26.04. In other embodiments, the X-ray powder diffraction pattern comprises the peak positions, in degrees 2-theta (±0.2 degrees 2-theta), set forth in Table 15. In other embodiments, the X-ray powder diffraction pattern is similar to the X-ray powder diffraction pattern shown in FIG. 25.

In one aspect, the cocrystal comprising the compound of formula (I) is a D-gluconic acid cocrystal.

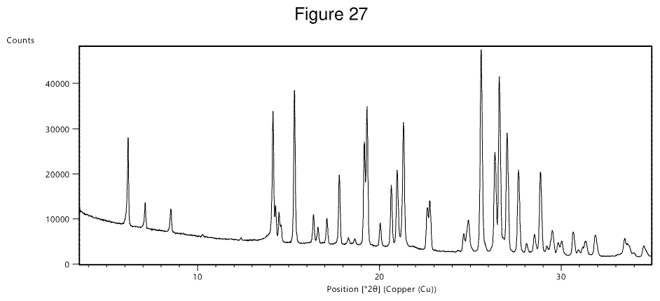

In some embodiments, the D-gluconic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 16 below. In other embodiments, the X-ray powder diffraction pattern comprises at least one peak position, in degrees 2-theta (±0.2 degrees 2-theta), selected from the group consisting of 6.17, 14.14, 15.32, 19.17 and 19.32. In other embodiments, the X-ray powder diffraction pattern comprises at least two peak positions, in degrees 2-theta (±0.2 degrees 2-theta), selected from the group consisting of 6.17, 14.14, 15.32, 19.17 and 19.32. In other embodiments, the X-ray powder diffraction pattern comprises at least three peak positions, in degrees 2-theta (±0.2 degrees 2-theta), selected from the group consisting of 6.17, 14.14, 15.32, 19.17 and 19.32. In other embodiments, the X-ray powder diffraction comprises at least four peak positions, in degrees 2-theta (±0.2 degrees 2-theta), selected from the group consisting of 6.17, 14.14, 15.32, 19.17 and 19.32. In other embodiments, the X-ray powder diffraction pattern comprises peak positions, in degrees 2-theta (±0.2 degrees 2-theta), of 15.32 and 19.32, and at least one peak position selected from the group consisting of 6.17, 14.14 and 19.17. In other embodiments, the X-ray powder diffraction pattern comprises the peak positions, in degrees 2-theta (±0.2 degrees 2-theta), set forth in Table 16. In other embodiments, the X-ray powder diffraction pattern is similar to the X-ray powder diffraction pattern shown in FIG. 27.

In one aspect, the cocrystal comprising the compound of formula (I) is a glycolic acid cocrystal.

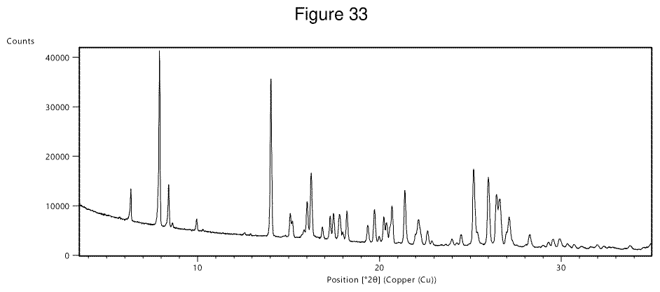

In some embodiments, the glycolic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 17 below. In other embodiments, the X-ray powder diffraction pattern comprises at least one peak position, in degrees 2-theta (±0.2 degrees 2-theta), selected from the group consisting of 7.90, 14.04 and 16.25. In other embodiments, the X-ray powder diffraction pattern comprises at least two peak positions, in degrees 2-theta (±0.2 degrees 2-theta), selected from the group consisting of 7.90, 14.04 and 16.25. In other embodiments, the X-ray powder diffraction pattern comprises the three following peak positions, in degrees 2-theta (±0.2 degrees 2-theta): 7.90, 14.04 and 16.25. In other embodiments, the X-ray powder diffraction pattern comprises the peak positions, in degrees 2-theta (±0.2 degrees 2-theta), set forth in Table 17. In other embodiments, the X-ray powder diffraction pattern is similar to the X-ray powder diffraction pattern shown in FIG. 33.

In one aspect, the cocrystal comprising the compound of formula (I) is a L-malic acid cocrystal.

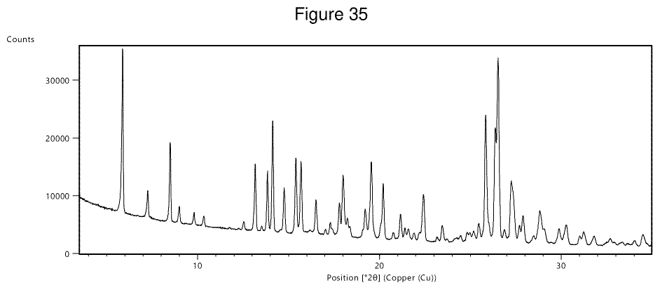

In some embodiments, the L-malic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 18 below. In other embodiments, the X-ray powder diffraction pattern comprises at least one peak position, in degrees 2-theta (±0.2 degrees 2-theta), selected from the group consisting of 5.87, 8.49, 14.13, 15.40 and 19.55. In other embodiments, the X-ray powder diffraction pattern comprises at least two peak positions, in degrees 2-theta (±0.2 degrees 2-theta), selected from the group consisting of 5.87, 8.49, 14.13, 15.40 and 19.55. In other embodiments, the X-ray powder diffraction pattern comprises at least three peak positions, in degrees 2-theta (±0.2 degrees 2-theta) selected from the group consisting of 5.87, 8.49, 14.13, 15.40 and 19.55. In other embodiments, the X-ray powder diffraction pattern comprises at least four peak positions, in degrees 2-theta (±0.2 degrees 2-theta) selected from the group consisting of 5.87, 8.49, 14.13, 15.40 and 19.55. In other embodiments, the X-ray powder diffraction pattern comprises peak positions, in degrees 2-theta (±0.2 degrees 2-theta), of 5.87 and 14.13, and at least one peak position selected from the group consisting of 8.49, 15.40 and 19.55. In other embodiments, the X-ray powder diffraction pattern comprises the peak positions, in degrees 2-theta (±0.2 degrees 2-theta), set forth in Table 18. In other embodiments, the X-ray powder diffraction pattern is similar to the X-ray powder diffraction pattern shown in FIG. 35.

In one aspect, the cocrystal comprising the compound of formula (I) is an oxalic acid cocrystal.

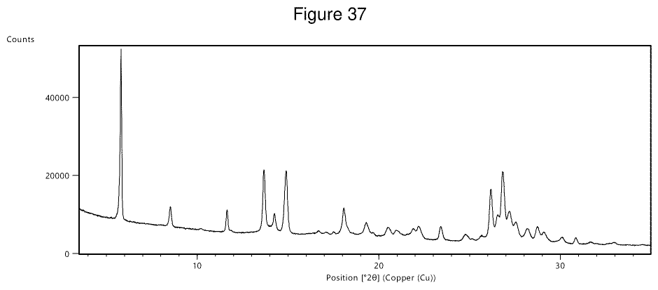

In some embodiments, the oxalic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 19 below. In other embodiments, the X-ray powder diffraction pattern comprises at least one peak position, in degrees 2-theta (±0.2 degrees 2-theta), selected from the group consisting of 5.81, 13.67 and 14.90. In other embodiments, the X-ray powder diffraction pattern comprises at least two peak positions, in degrees 2-theta (±0.2 degrees 2-theta), selected from the group consisting of 5.81, 13.67 and 14.90. In other embodiments, the X-ray powder diffraction pattern comprises the three following peak positions, in degrees 2-theta (±0.2 degrees 2-theta): 5.81, 13.67 and 14.90. In other embodiments, the X-ray powder diffraction pattern comprises the peak positions, in degrees 2-theta (±0.2 degrees 2-theta), set forth in Table 19. In other embodiments, the X-ray powder diffraction pattern is similar to the X-ray powder diffraction pattern shown in FIG. 37.

In one aspect, the cocrystal comprising the compound of formula (I) is a benzoic acid cocrystal.

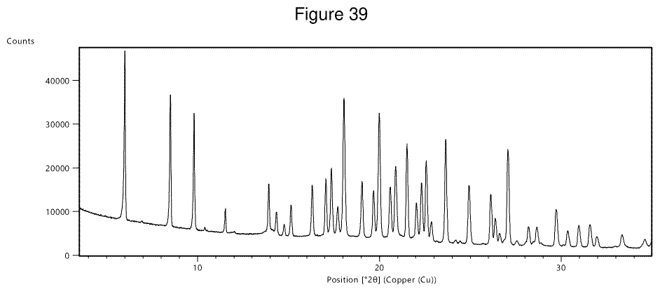

In some embodiments, the benzoic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 20 below. In other embodiments, the X-ray powder diffraction pattern comprises at least one peak position, in degrees 2-theta (±0.2 degrees 2-theta), selected from the group consisting of 6.00, 8.50, 9.80, 18.05, and 19.99. In other embodiments, the X-ray powder diffraction pattern comprises at least two peak positions, in degrees 2-theta (±0.2 degrees 2-theta), selected from the group consisting of 6.00, 8.50, 9.80, 18.05, and 19.99. In other embodiments, the X-ray powder diffraction pattern comprises at least three peak positions, in degrees 2-theta (±0.2 degrees 2-theta) selected from the group consisting of 6.00, 8.50, 9.80, 18.05, and 19.99. In other embodiments, the X-ray powder diffraction pattern comprises at least four peak positions, in degrees 2-theta (±0.2 degrees 2-theta) selected from the group consisting of 6.00, 8.50, 9.80, 18.05, and 19.99. In other embodiments, the X-ray powder diffraction pattern comprises peak positions, in degrees 2-theta (±0.2 degrees 2-theta), of 6.00 and 18.05, and at least one peak position selected from the group consisting of 8.50, 9.80, and 19.99. In other embodiments, the X-ray powder diffraction pattern comprises the peak positions, in degrees 2-theta (±0.2 degrees 2-theta), set forth in Table 20. In other embodiments, the X-ray powder diffraction pattern is similar to the X-ray powder diffraction pattern shown in FIG. 39.

In some embodiments, the benzoic acid cocrystal of the compound of formula (I) is characterized by a differential scanning calorimetry thermogram comprising an endothermic peak having an onset temperature of 128.06° C. (+5.0° C.). In other embodiments, the benzoic acid cocrystal of the compound of formula (I) is characterized by a differential scanning calorimetry thermogram comprising an endothermic peak having an onset temperature of 128.06° C. (±2.0° C.).

In one aspect, the cocrystal comprising the compound of formula (I) is a fumaric acid cocrystal.

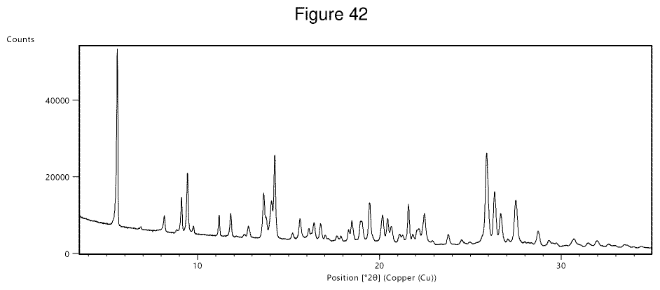

In some embodiments, the fumaric acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 21 below. In other embodiments, the X-ray powder diffraction pattern comprises at least one peak position, in degrees 2-theta (±0.2 degrees 2-theta), selected from the group consisting of 5.59, 9.45, 13.63, 14.25 and 19.46. In other embodiments, the X-ray powder diffraction pattern comprises at least two peak positions, in degrees 2-theta (±0.2 degrees 2-theta), selected from the group consisting of 5.59, 9.45, 13.63, 14.25 and 19.46. In other embodiments, the X-ray powder diffraction pattern comprises at least three peak positions, in degrees 2-theta (±0.2 degrees 2-theta) selected from the group consisting of 5.59, 9.45, 13.63, 14.25 and 19.46. In other embodiments, the X-ray powder diffraction pattern comprises at least four peak positions, in degrees 2-theta (±0.2 degrees 2-theta) selected from the group consisting of 5.59, 9.45, 13.63, 14.25 and 19.46. In other embodiments, the X-ray powder diffraction pattern comprises peak positions, in degrees 2-theta (±0.2 degrees 2-theta), of 5.59 and 14.25, and at least one peak position selected from the group consisting of 9.45, 13.36 and 19.46. In other embodiments, the X-ray powder diffraction pattern comprises the peak positions, in degrees 2-theta (±0.2 degrees 2-theta), set forth in Table 21. In other embodiments, the X-ray powder diffraction pattern is similar to the X-ray powder diffraction pattern shown in FIG. 42.

In one aspect, the cocrystal comprising the compound of formula (I) is a gentisic acid cocrystal.

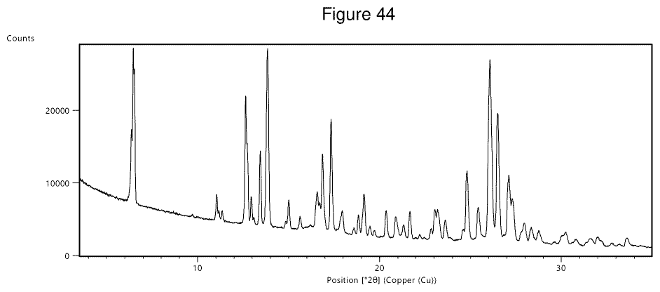

In some embodiments, the gentisic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 22 below. In other embodiments, the X-ray powder diffraction pattern comprises at least one peak position, in degrees 2-theta (±0.2 degrees 2-theta), selected from the group consisting of 6.46, 12.63, 13.84, 17.34 and 26.08. In other embodiments, the X-ray powder diffraction pattern comprises at least two peak positions, in degrees 2-theta (±0.2 degrees 2-theta), selected from the group consisting of 6.46, 12.63, 13.84, 17.34 and 26.08. In other embodiments, the X-ray powder diffraction pattern comprises at least three peak positions, in degrees 2-theta (±0.2 degrees 2-theta) selected from the group consisting of 6.46, 12.63, 13.84, 17.34 and 26.08. In other embodiments, the X-ray powder diffraction pattern comprises at least four peak positions, in degrees 2-theta (±0.2 degrees 2-theta) selected from the group consisting of 6.46, 12.63, 13.84, 17.34 and 26.08. In other embodiments, the X-ray powder diffraction pattern comprises peak positions, in degrees 2-theta (±0.2 degrees 2-theta), of 13.84 and 26.08, and at least one peak position selected from the group consisting of 6.46, 12.63 and 17.34. In other embodiments, the X-ray powder diffraction pattern comprises the peak positions, in degrees 2-theta (±0.2 degrees 2-theta), set forth in Table 22. In other embodiments, the X-ray powder diffraction pattern is similar to the X-ray powder diffraction pattern shown in FIG. 44.

In one aspect, the cocrystal comprising the compound of formula (I) is a glutaric acid cocrystal.

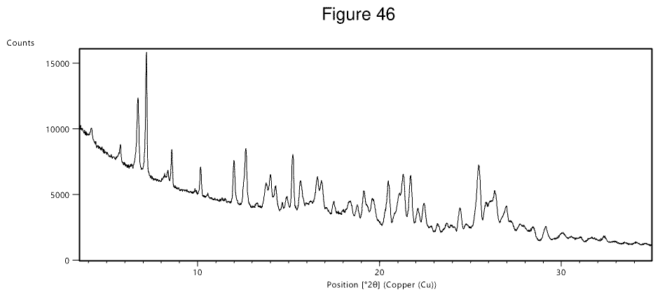

In some embodiments, the glutaric acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 23 below. In other embodiments, the X-ray powder diffraction pattern comprises at least one peak position, in degrees 2-theta (±0.2 degrees 2-theta), selected from the group consisting of 6.72, 7.19, 12.00, 12.65 and 15.23. In other embodiments, the X-ray powder diffraction pattern comprises at least two peak positions, in degrees 2-theta (±0.2 degrees 2-theta), selected from the group consisting of 6.72, 7.19, 12.00, 12.65 and 15.23. In other embodiments, the X-ray powder diffraction pattern comprises at least three peak positions, in degrees 2-theta (±0.2 degrees 2-theta) selected from the group consisting of 6.72, 7.19, 12.00, 12.65 and 15.23. In other embodiments, the X-ray powder diffraction pattern comprises at least four peak positions, in degrees 2-theta (±0.2 degrees 2-theta) selected from the group consisting of 6.72, 7.19, 12.00, 12.65 and 15.23. In other embodiments, the X-ray powder diffraction pattern comprises peak positions, in degrees 2-theta (±0.2 degrees 2-theta), of 6.72 and 7.19, and at least one peak position selected from the group consisting of 12.00, 12.65 and 15.23. In other embodiments, the X-ray powder diffraction pattern comprises the peak positions, in degrees 2-theta (±0.2 degrees 2-theta), set forth in Table 23. In other embodiments, the X-ray powder diffraction pattern is similar to the X-ray powder diffraction pattern shown in FIG. 46.

In one aspect, the cocrystal comprising the compound of formula (I) is a 4-hydroxybenzoic acid cocrystal.

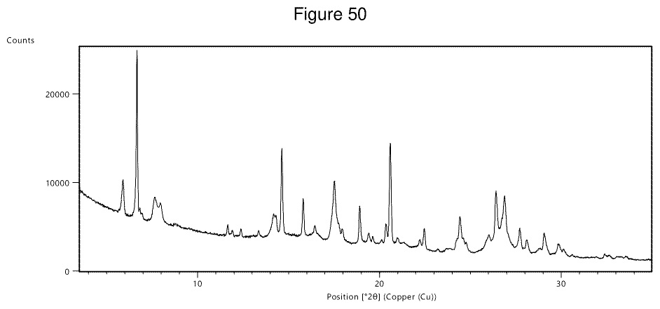

In some embodiments, the 4-hydroxybenzoic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 25 below. In other embodiments, the X-ray powder diffraction pattern comprises at least one peak position, in degrees 2-theta (±0.2 degrees 2-theta), selected from the group consisting of 6.67, 14.64, 17.52, 20.59, 26.40 and 26.88. In other embodiments, the X-ray powder diffraction pattern comprises at least two peak positions, in degrees 2-theta (±0.2 degrees 2-theta), selected from the group consisting of 6.67, 14.64, 17.52, 20.59, 26.40 and 26.88. In other embodiments, the X-ray powder diffraction pattern comprises at least three peak positions, in degrees 2-theta (±0.2 degrees 2-theta) selected from the group consisting of 6.67, 14.64, 17.52, 20.59, 26.40 and 26.88. In other embodiments, the X-ray powder diffraction pattern comprises at least four peak positions, in degrees 2-theta (±0.2 degrees 2-theta) selected from the group consisting of 6.67, 14.64, 17.52, 20.59, 26.40 and 26.88. In other embodiments, the X-ray powder diffraction pattern comprises peak positions, in degrees 2-theta (±0.2 degrees 2-theta), of 6.67 and 14.64, and at least one peak position selected from the group consisting of 17.52, 20.59, 26.40 and 26.88. In other embodiments, the X-ray powder diffraction pattern comprises the peak positions, in degrees 2-theta (±0.2 degrees 2-theta), set forth in Table 25. In other embodiments, the X-ray powder diffraction pattern is similar to the X-ray powder diffraction pattern shown in FIG. 50.

In one aspect, the cocrystal comprising the compound of formula (I) is an alpha-ketoglutaric acid cocrystal.

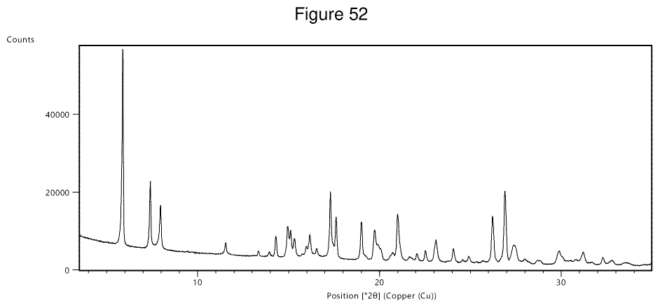

In some embodiments, the alpha-ketoglutaric acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 26 below. In other embodiments, the X-ray powder diffraction pattern comprises at least one peak position, in degrees 2-theta (±0.2 degrees 2-theta), selected from the group consisting of 5.89, 7.39, 17.30 and 26.89. In other embodiments, the X-ray powder diffraction pattern comprises at least two peak positions, in degrees 2-theta (±0.2 degrees 2-theta), selected from the group consisting of 5.89, 7.39, 17.30 and 26.89. In other embodiments, the X-ray powder diffraction pattern comprises at least three peak positions, in degrees 2-theta (±0.2 degrees 2-theta) selected from the group consisting of 5.89, 7.39, 17.30 and 26.89. In other embodiments, the X-ray powder diffraction pattern comprises the four following peak positions, in degrees 2-theta (±0.2 degrees 2-theta) selected from the group consisting of 5.89, 7.39, 17.30 and 26.89. In other embodiments, the X-ray powder diffraction pattern comprises peak positions, in degrees 2-theta (±0.2 degrees 2-theta), of 5.89 and 17.30, and at least one peak position selected from the group consisting of 7.39 and 26.89. In other embodiments, the X-ray powder diffraction pattern comprises the peak positions, in degrees 2-theta (±0.2 degrees 2-theta), set forth in Table 26. In other embodiments, the X-ray powder diffraction pattern is similar to the X-ray powder diffraction pattern shown in FIG. 52.

In one aspect, the cocrystal comprising the compound of formula (I) is a malonic acid cocrystal.

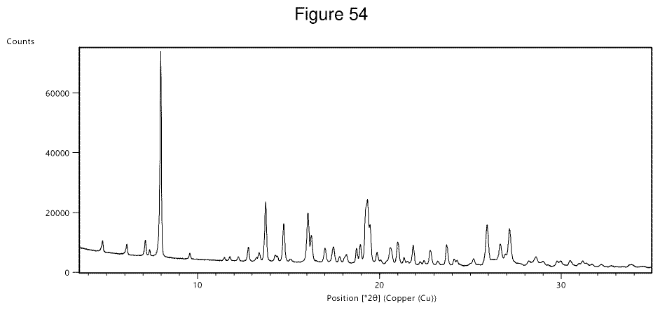

In some embodiments, the malonic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 27 below. In other embodiments, the X-ray powder diffraction pattern comprises at least one peak position, in degrees 2-theta (±0.2 degrees 2-theta), selected from the group consisting of 7.97, 13.74, 16.07 and 19.34. In other embodiments, the X-ray powder diffraction pattern comprises at least two peak positions, in degrees 2-theta (±0.2 degrees 2-theta), selected from the group consisting of 7.97, 13.74, 16.07 and 19.34. In other embodiments, the X-ray powder diffraction pattern comprises at least three peak positions, in degrees 2-theta (±0.2 degrees 2-theta) selected from the group consisting of 7.97, 13.74, 16.07 and 19.34. In other embodiments, the X-ray powder diffraction pattern comprises the following four peak positions, in degrees 2-theta (±0.2 degrees 2-theta) selected from the group consisting of 7.97, 13.74, 16.07 and 19.34. In other embodiments, the X-ray powder diffraction pattern comprises peak positions, in degrees 2-theta (±0.2 degrees 2-theta), of 7.97 and 19.34, and at least one peak position selected from the group consisting of 13.74 and 16.07. In other embodiments, the X-ray powder diffraction pattern comprises the peak positions, in degrees 2-theta (±0.2 degrees 2-theta), set forth in Table 27. In other embodiments, the X-ray powder diffraction pattern is similar to the X-ray powder diffraction pattern shown in FIG. 54.

In one aspect, the cocrystal comprising the compound of formula (I) is a salicylic acid cocrystal.

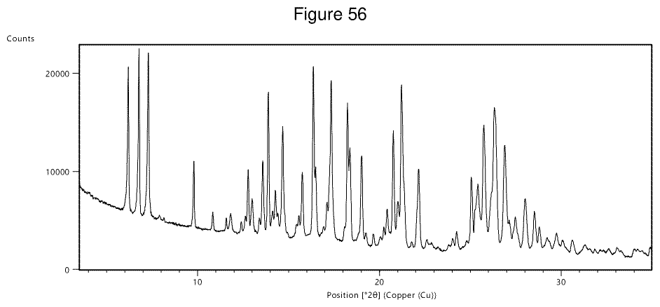

In some embodiments, the salicylic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 28 below. In other embodiments, the X-ray powder diffraction pattern comprises at least one peak position, in degrees 2-theta (±0.2 degrees 2-theta), selected from the group consisting of 6.77, 7.29, 16.36, 18.24 and 21.21. In other embodiments, the X-ray powder diffraction pattern comprises at least two peak positions, in degrees 2-theta (±0.2 degrees 2-theta), selected from the group consisting of 6.77, 7.29, 16.36, 18.24 and 21.21. In other embodiments, the X-ray powder diffraction pattern comprises at least three peak positions, in degrees 2-theta (±0.2 degrees 2-theta) selected from the group consisting of 6.77, 7.29, 16.36, 18.24 and 21.21. In other embodiments, the X-ray powder diffraction pattern comprises at least four peak positions, in degrees 2-theta (±0.2 degrees 2-theta) selected from the group consisting of 6.77, 7.29, 16.36, 18.24 and 21.21. In other embodiments, the X-ray powder diffraction pattern comprises peak positions, in degrees 2-theta (±0.2 degrees 2-theta), of 6.77 and 16.36, and at least one peak position selected from the group consisting of 7.29, 18.24 and 21.21. In other embodiments, the X-ray powder diffraction pattern comprises the peak positions, in degrees 2-theta (±0.2 degrees 2-theta), set forth in Table 28. In other embodiments, the X-ray powder diffraction pattern is similar to the X-ray powder diffraction pattern shown in FIG. 56.

In one aspect, the cocrystal comprising the compound of formula (I) is a L-tartaric acid cocrystal.

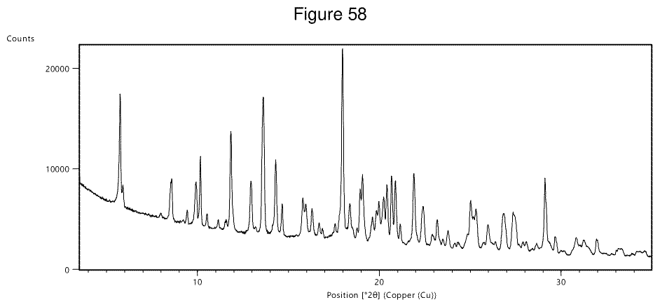

In some embodiments, the L-tartaric acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 29 below. In other embodiments, the X-ray powder diffraction pattern comprises at least one peak position, in degrees 2-theta (±0.2 degrees 2-theta), selected from the group consisting of 5.74, 11.84, 13.62 and 17.98. In other embodiments, the X-ray powder diffraction pattern comprises at least two peak positions, in degrees 2-theta (±0.2 degrees 2-theta), selected from the group consisting of 5.74, 11.84, 13.62 and 17.98. In other embodiments, the X-ray powder diffraction pattern comprises at least three peak positions, in degrees 2-theta (±0.2 degrees 2-theta) selected from the group consisting of 5.74, 11.84, 13.62 and 17.98. In other embodiments, the X-ray powder diffraction pattern comprises the following four peak positions, in degrees 2-theta (±0.2 degrees 2-theta) selected from the group consisting of 5.74, 11.84, 13.62 and 17.98. In other embodiments, the X-ray powder diffraction pattern comprises peak positions, in degrees 2-theta (±0.2 degrees 2-theta), of 13.62 and 17.98, and at least one peak position selected from the group consisting of 5.74 and 11.84. In other embodiments, the X-ray powder diffraction pattern comprises the peak positions, in degrees 2-theta (±0.2 degrees 2-theta), set forth in Table 29. In other embodiments, the X-ray powder diffraction pattern is similar to the X-ray powder diffraction pattern shown in FIG. 58.

In one aspect, the cocrystal comprising the compound of formula (I) is a urea cocrystal.

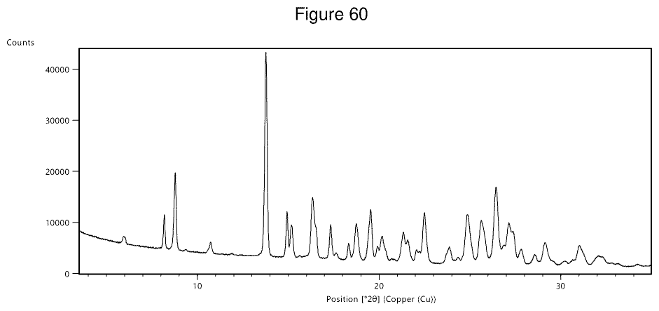

In some embodiments, the urea cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 30 below. In other embodiments, the X-ray powder diffraction pattern comprises at least one peak position, in degrees 2-theta (±0.2 degrees 2-theta), selected from the group consisting of 8.79, 13.76, 16.34 and 26.43. In other embodiments, the X-ray powder diffraction pattern comprises at least two peak positions, in degrees 2-theta (±0.2 degrees 2-theta), selected from the group consisting of 8.79, 13.76, 16.34 and 26.43. In other embodiments, the X-ray powder diffraction pattern comprises at least three peak positions, in degrees 2-theta (±0.2 degrees 2-theta) selected from the group consisting of 8.79, 13.76, 16.34 and 26.43. In other embodiments, the X-ray powder diffraction pattern comprises the following four peak positions, in degrees 2-theta (±0.2 degrees 2-theta) selected from the group consisting of 8.79, 13.76, 16.34 and 26.43. In other embodiments, the X-ray powder diffraction pattern comprises peak positions, in degrees 2-theta (±0.2 degrees 2-theta), of 8.79 and 13.76, and at least one peak position selected from the group consisting of 16.34 and 26.43. In other embodiments, the X-ray powder diffraction pattern comprises the peak positions, in degrees 2-theta (±0.2 degrees 2-theta), set forth in Table 30. In other embodiments, the X-ray powder diffraction pattern is similar to the X-ray powder diffraction pattern shown in FIG. 60.

In one aspect, the cocrystal comprising the compound of formula (I) is a pyroglutamic acid cocrystal.

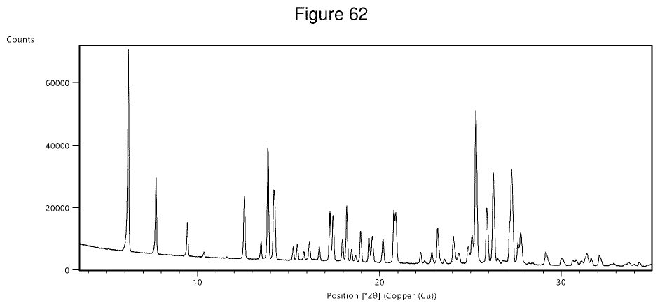

In some embodiments, the pyroglutamic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 31 below. In other embodiments, the X-ray powder diffraction pattern comprises at least one peak position, in degrees 2-theta (±0.2 degrees 2-theta), selected from the group consisting of 6.20, 13.87, 25.30, 26.24 and 27.26. In other embodiments, the X-ray powder diffraction pattern comprises at least two peak positions, in degrees 2-theta (±0.2 degrees 2-theta), selected from the group consisting of 6.20, 13.87, 25.30, 26.24 and 27.26. In other embodiments, the X-ray powder diffraction pattern comprises at least three peak positions, in degrees 2-theta (±0.2 degrees 2-theta) selected from the group consisting of 6.20, 13.87, 25.30, 26.24 and 27.26. In other embodiments, the X-ray powder diffraction pattern comprises at least four peak positions, in degrees 2-theta (±0.2 degrees 2-theta) selected from the group consisting of 6.20, 13.87, 25.30, 26.24 and 27.26. In other embodiments, the X-ray powder diffraction pattern comprises peak positions, in degrees 2-theta (±0.2 degrees 2-theta), of 6.20 and 13.87, and at least one peak position selected from the group consisting of 25.30, 26.24 and 27.26. In other embodiments, the X-ray powder diffraction pattern comprises the peak positions, in degrees 2-theta (±0.2 degrees 2-theta), set forth in Table 31. In other embodiments, the X-ray powder diffraction pattern is similar to the X-ray powder diffraction pattern shown in FIG. 62.

In one aspect, the cocrystal comprising the compound of formula (I) is a caproic acid cocrystal.

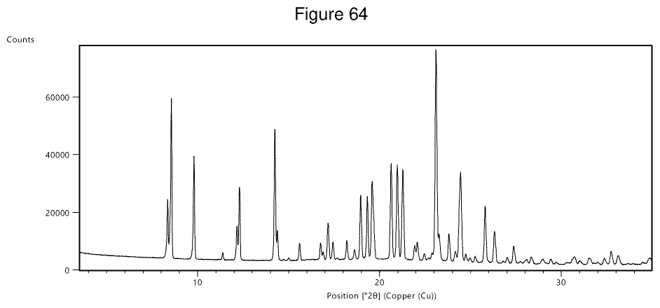

In some embodiments, the caproic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 32 below. In other embodiments, the X-ray powder diffraction pattern comprises at least one peak position, in degrees 2-theta (±0.2 degrees 2-theta), selected from the group consisting of 8.55, 9.80, 14.25 and 23.10. In other embodiments, the X-ray powder diffraction pattern comprises at least two peak positions, in degrees 2-theta (±0.2 degrees 2-theta), selected from the group consisting of 8.55, 9.80, 14.25 and 23.10. In other embodiments, the X-ray powder diffraction pattern comprises at least three peak positions, in degrees 2-theta (±0.2 degrees 2-theta) selected from the group consisting of 8.55, 9.80, 14.25 and 23.10. In other embodiments, the X-ray powder diffraction pattern comprises the following four peak positions, in degrees 2-theta (±0.2 degrees 2-theta) selected from the group consisting of 8.55, 9.80, 14.25 and 23.10. In other embodiments, the X-ray powder diffraction pattern comprises peak positions, in degrees 2-theta (±0.2 degrees 2-theta), of 8.55 and 14.25, and at least one peak position selected from the group consisting of 9.80, and 23.10. In other embodiments, the X-ray powder diffraction pattern comprises the peak positions, in degrees 2-theta (±0.2 degrees 2-theta), set forth in Table 32. In other embodiments, the X-ray powder diffraction pattern is similar to the X-ray powder diffraction pattern shown in FIG. 64.

In one aspect, the cocrystal comprising the compound of formula (I) is a glycerol cocrystal.

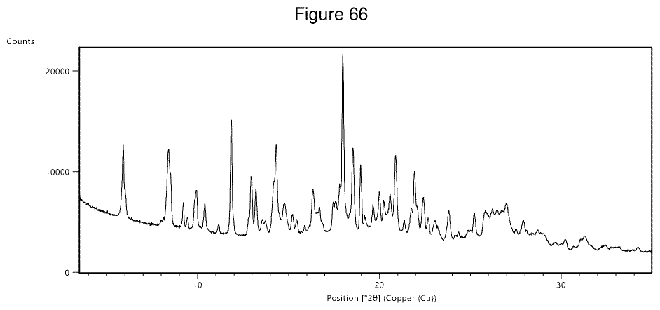

In some embodiments, the glycerol cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 33 below. In other embodiments, the X-ray powder diffraction pattern comprises at least one peak position, in degrees 2-theta (±0.2 degrees 2-theta), selected from the group consisting of 5.91, 8.41, 11.85, 14.32 and 17.99. In other embodiments, the X-ray powder diffraction pattern comprises at least two peak positions, in degrees 2-theta (±0.2 degrees 2-theta), selected from the group consisting of 5.91, 8.41, 11.85, 14.32 and 17.99. In other embodiments, the X-ray powder diffraction pattern comprises at least three peak positions, in degrees 2-theta (±0.2 degrees 2-theta) selected from the group consisting of 5.91, 8.41, 11.85, 14.32 and 17.99. In other embodiments, the X-ray powder diffraction pattern comprises at least four peak positions, in degrees 2-theta (±0.2 degrees 2-theta) selected from the group consisting of 5.91, 8.41, 11.85, 14.32 and 17.99. In other embodiments, the X-ray powder diffraction pattern comprises peak positions, in degrees 2-theta (±0.2 degrees 2-theta), of 11.85 and 17.99, and at least one peak position selected from the group consisting of 5.91, 8.41 and 14.32. In other embodiments, the X-ray powder diffraction pattern comprises the peak positions, in degrees 2-theta (±0.2 degrees 2-theta), set forth in Table 33. In other embodiments, the X-ray powder diffraction pattern is similar to the X-ray powder diffraction pattern shown in FIG. 66.

In one aspect, the cocrystal comprising the compound of formula (I) is a L-lysine cocrystal.

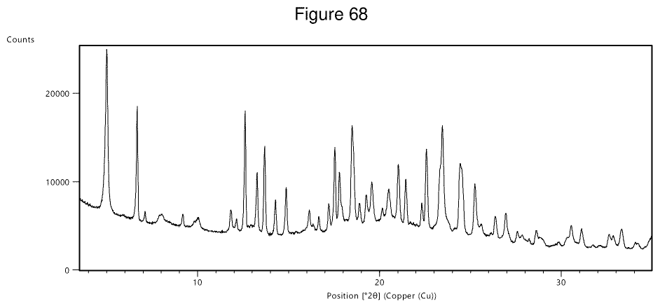

In some embodiments, the L-lysine cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 34 below. In other embodiments, the X-ray powder diffraction pattern comprises at least one peak position, in degrees 2-theta (±0.2 degrees 2-theta), selected from the group consisting of 5.00, 6.68, 12.61, 18.50 and 23.45. In other embodiments, the X-ray powder diffraction pattern comprises at least two peak positions, in degrees 2-theta (±0.2 degrees 2-theta), selected from the group consisting of 5.00, 6.68, 12.61, 18.50 and 23.45. In other embodiments, the X-ray powder diffraction pattern comprises at least three peak positions, in degrees 2-theta (±0.2 degrees 2-theta) selected from the group consisting of 5.00, 6.68, 12.61, 18.50 and 23.45. In other embodiments, the X-ray powder diffraction pattern comprises at least four peak positions, in degrees 2-theta (±0.2 degrees 2-theta) selected from the group consisting of 5.00, 6.68, 12.61, 18.50 and 23.45. In other embodiments, the X-ray powder diffraction pattern comprises peak positions, in degrees 2-theta (±0.2 degrees 2-theta), of 5.00 and 18.50, and at least one peak position selected from the group consisting of 6.68, 12.61 and 23.45. In other embodiments, the X-ray powder diffraction pattern comprises the peak positions, in degrees 2-theta (±0.2 degrees 2-theta), set forth in Table 34. In other embodiments, the X-ray powder diffraction pattern is similar to the X-ray powder diffraction pattern shown in FIG. 68.

In one aspect, the cocrystal comprising the compound of formula (I) is a S-proline cocrystal.

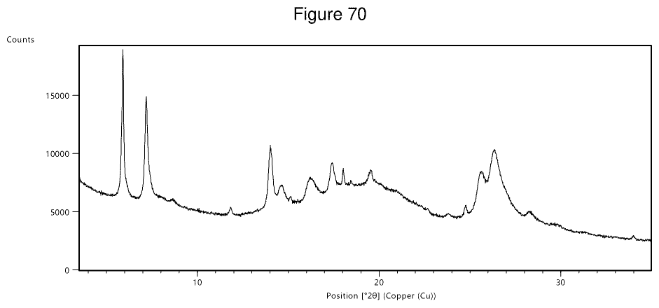

In some embodiments, the S-proline cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 35 below. In other embodiments, the X-ray powder diffraction pattern comprises at least one peak position, in degrees 2-theta (±0.2 degrees 2-theta), selected from the group consisting of 5.91, 7.19, 14.02 and 26.36. In other embodiments, the X-ray powder diffraction pattern comprises at least two peak positions, in degrees 2-theta (±0.2 degrees 2-theta), selected from the group consisting of 5.91, 7.19, 14.02 and 26.36. In other embodiments, the X-ray powder diffraction pattern comprises at least three peak positions, in degrees 2-theta (±0.2 degrees 2-theta) selected from the group consisting of 5.91, 7.19, 14.02 and 26.36. In other embodiments, the X-ray powder diffraction pattern comprises the following four peak positions, in degrees 2-theta (±0.2 degrees 2-theta) selected from the group consisting of 5.91, 7.19, 14.02 and 26.36. In other embodiments, the X-ray powder diffraction pattern comprises peak positions, in degrees 2-theta (±0.2 degrees 2-theta), of 5.91 and 7.19, and at least one peak position selected from the group consisting of 14.02 and 26.36. In other embodiments, the X-ray powder diffraction pattern comprises the peak positions, in degrees 2-theta (±0.2 degrees 2-theta), set forth in Table 35. In other embodiments, the X-ray powder diffraction pattern is similar to the X-ray powder diffraction pattern shown in FIG. 70.

In one aspect, the cocrystal comprising the compound of formula (I) is a pyruvic acid cocrystal.

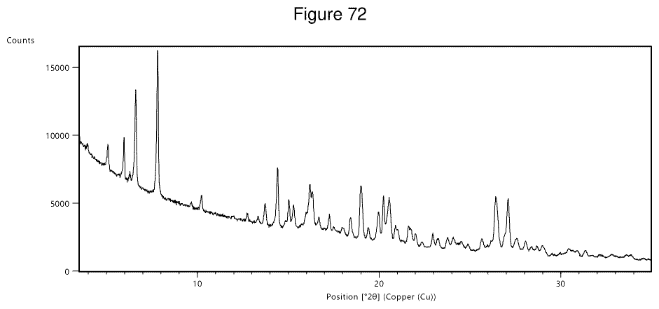

In some embodiments, the pyruvic acid cocrystal of the compound of formula (I) is characterized by an X-ray powder diffraction pattern, acquired in reflection mode (sometimes referred to as reflectance mode), comprising one or more peak positions, in degrees 2-theta (±0.2 degrees 2-theta), selected from the peak positions set forth in Table 36 below. In other embodiments, the X-ray powder diffraction pattern comprises at least one peak position, in degrees 2-theta (±0.2 degrees 2-theta), selected from the group consisting of 6.61, 7.81, 14.40, 18.99, 26.41 and 27.10. In other embodiments, the X-ray powder diffraction pattern comprises at least two peak positions, in degrees 2-theta (±0.2 degrees 2-theta), selected from the group consisting of 6.61, 7.81, 14.40, 18.99, 26.41 and 27.10. In other embodiments, the X-ray powder diffraction pattern comprises at least three peak positions, in degrees 2-theta (±0.2 degrees 2-theta) selected from the group consisting of 6.61, 7.81, 14.40, 18.99, 26.41 and 27.10. In other embodiments, the X-ray powder diffraction pattern comprises at least four peak positions, in degrees 2-theta (±0.2 degrees 2-theta) selected from the group consisting of 6.61, 7.81, 14.40, 18.99, 26.41 and 27.10. In other embodiments, the X-ray powder diffraction pattern comprises peak positions, in degrees 2-theta (±0.2 degrees 2-theta), of 6.61 and 7.81, and at least one peak position selected from the group consisting of 14.40, 18.99, 26.41 and 27.10. In other embodiments, the X-ray powder diffraction pattern comprises the peak positions, in degrees 2-theta (±0.2 degrees 2-theta), set forth in Table 36. In other embodiments, the X-ray powder diffraction pattern is similar to the X-ray powder diffraction pattern shown in FIG. 72.

As used herein, the term “unit cell” refers to the smallest group of particles (e.g., molecules) in a crystalline solid that makes up the repeating pattern of the crystalline solid. In a cocrystal, the term “unit cell” refers to the smallest group of the two or more neutral chemical species that makes up the repeating pattern of the cocrystal.

In another aspect, the disclosure relates to an amorphous solid dispersion comprising a compound of formula (I) or prepared from a pharmaceutically acceptable salt or a cocrystal thereof

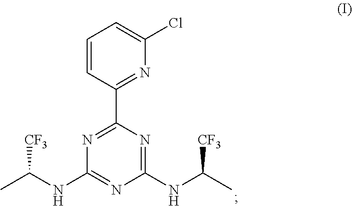 [SMILES: C[C@@H](Nc1nc(N[C@H](C)C(F)(F)F)nc(-c2cccc(Cl)n2)n1)C(F)(F)F]

and a polymer.

As used herein, the term “dispersion” refers to a disperse system in which one substance (the dispersed phase) is distributed, in discrete units, throughout a second substance (the continuous phase or vehicle). In general, the dispersed phases can be solids, liquids, or gases. In the case of a solid dispersion, the dispersed and continuous phases are both solids.

As used herein, the term “amorphous solid dispersion” generally refers to a solid dispersion of two or more components, usually a therapeutically active compound and a polymer (or plurality of polymers), but possibly containing other components such as surfactants or other pharmaceutical excipients, where the therapeutically active compound is in the amorphous phase. In some embodiments, an amorphous solid dispersion includes the polymer(s) (and optionally a surfactant) constituting the dispersed phase, and the therapeutically active compound constitutes the continuous phase. In some embodiments, an amorphous solid dispersion includes the polymer(s) (and optionally a surfactant) constituting the continuous phase, and the therapeutically active compound constitutes the dispersed phase.

In some embodiments, the polymer is selected from the group consisting of hydroxypropyl methyl cellulose (HPMC), hydroxypropyl methylcellulose acetate succinate (HPMCAS), hydroxypropyl methyl cellulose phthalate (HPMCP), hydroxypropyl cellulose (HPC), ethylcellulose, cellulose acetate phthalate, and polyvinylpyrrolidone (PVP), or a mixture thereof. In other embodiments, the polymer is HPMCAS.

In some embodiments, the polymer is present in the amorphous solid dispersion in an amount of between about 10% w/w and 90% w/w (e.g., between about 20% w/w and about 80% w/w; between about 30% w/w and about 70% w/w; between about 40% w/w and about 60% w/w; or between about 15% w/w and about 35% w/w). In some embodiments, the polymer is (or the one or more polymers are) present in the amorphous solid dispersion in an amount of from about 10% w/w to about 80% w/w, for example from about 30% w/w to about 75% w/w, or from about 40% w/w to about 65% w/w, or from about 45% w/w to about 55% w/w, for example, about 46% w/w, about 47% w/w, about 48% w/w, about 49% w/w, about 50% w/w, about 51% w/w, about 52% w/w, about 53% w/w, or about 54% w/w. In some embodiments, the polymer is (or the one or more polymers are) present in the amorphous solid dispersion in an amount of about 48% w/w, about 48.5% w/w, about 49% w/w, about 49.5% w/w, about 50% w/w, about 50.5% w/w, about 51% w/w, about 51.5% w/w, about 52% w/w, or about 52.5% w/w.

In some embodiments, the compound of formula (I) or a pharmaceutically acceptable salt or a cocrystal thereof is present in the amorphous solid dispersion in an amount of from about 10% w/w and 90% w/w (e.g., between about 20% w/w and about 80% w/w; between about 30% w/w and about 70% w/w; between about 40% w/w and about 60% w/w; or between about 15% w/w and about 35% w/w). In some embodiments, the compound of formula (I) is present in the amorphous solid dispersion in an amount of from about 10% w/w to about 80% w/w, for example from about 30% w/w to about 75% w/w, or from about 40% w/w to about 65% w/w, or from about 45% w/w to about 55% w/w, for example, about 46% w/w, about 47% w/w, about 48% w/w, about 49% w/w, about 50% w/w, about 51% w/w, about 52% w/w, about 53% w/w, or about 54% w/w. In some embodiments, the compound of formula (I) is present in the amorphous solid dispersion in an amount of about 48% w/w, about 48.5% w/w, about 49% w/w, about 49.5% w/w, about 50% w/w, about 50.5% w/w, about 51% w/w, about 51.5% w/w, about 52% w/w, or about 52.5% w/w.

In some embodiments, the amorphous solid dispersion further comprises a surfactant. In some embodiments, the surfactant is selected from the group consisting of sodium lauryl sulfate (SLS), vitamin E or a derivative thereof (e.g., vitamin E TPGS), docusate Sodium, sodium dodecyl sulfate, polysorbates (such as Tween 20 and Tween 80), poloxamers (such as Poloxamer 335 and Poloxamer 407), glyceryl monooleate, Span 65, Span 25, Capryol 90, pluronic copolymers (e.g., Pluronic F108, Pluronic P-123), and mixtures thereof. In some embodiments, the surfactant is SLS.

In some embodiments, the surfactant is present in the amorphous solid dispersion in an amount of from about 0.1% w/w to about 10% w/w, for example from about 0.5% w/w to about 2% w/w, or from about 1% w/w to about 3% w/w, from about 1% w/w to about 4% w/w, or from about 1% w/w to about 5% w/w. In some embodiments, the surfactant is present in the solid dispersion in an amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, or about 1% w/w. In some embodiments, the surfactant is present in the solid dispersion in an amount of about 0.5% w/w, about 1% w/w, about 1.5% w/w, about 2% w/w, about 2.5% w/w, about 3% w/w, about 3.5% w/w, about 4% w/w, about 4.5% w/w, or about 5% w/w.

In some embodiments, the amorphous solid dispersion comprises the compound of formula (I) and HPMCAS. In some embodiments, the amorphous solid dispersion consists essentially of the compound of formula (I) and HPMCAS. In some embodiments, the amorphous solid dispersion consists of the compound of formula (I) and HPMCAS. In some embodiments, the compound of formula (I) and HPMCAS are present in a weight ratio of between about 3:1 and about 1:3, or between about 2:1 and about 1:2, or between about 1.5:1 and about 1:1.5. In some embodiments, the compound of formula (I) and HPMCAS are present in a weight ratio of about 1:1.

In some embodiments, the amorphous solid dispersion has a glass transition temperature (T_{g}) of at least about 80° C. In other embodiments, the amorphous solid dispersion has a Tg of between about 80° C. and about 130° C., between about 80° C. and about 120° C., between about 80° C. and about 100° C., or between about 80° C. and about 90° C.

The disclosure also relates to tautomers of the chemical structure identified as the compound of formula (I). Such tautomers include:

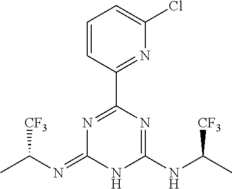 [SMILES: C[C@@H](/N=c1\nc(-c2cccc(Cl)n2)nc(N[C@H](C)C(F)(F)F)[nH]1)C(F)(F)F]

4-(6-chloropyridin-2-yl)-N—((R)-1,1,1-trifluoropropan-2-yl)-6-(((R)-1,1,1-trifluoropropan-2-yl)imino)-1,6-dihydro-1,3,5-triazin-2-amine;

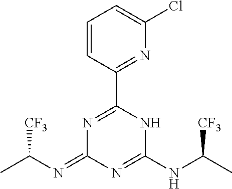 [SMILES: C[C@@H](/N=c1/nc(N[C@H](C)C(F)(F)F)[nH]c(-c2cccc(Cl)n2)n1)C(F)(F)F]

6-(6-chloropyridin-2-yl)-N—((R)-1,1,1-trifluoropropan-2-yl)-4-(((R)-1,1,1-trifluoropropan-2-yl)imino)-1,4-dihydro-1,3,5-triazin-2-amine;

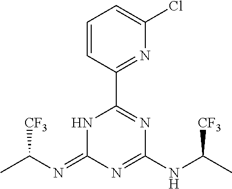 [SMILES: C[C@@H](/N=c1/nc(N[C@H](C)C(F)(F)F)nc(-c2cccc(Cl)n2)[nH]1)C(F)(F)F]

6-(6-chloropyridin-2-yl)-N—((R)-1,1,1-trifluoropropan-2-yl)-4-(((R)-1,1,1-trifluoropropan-2-yl)imino)-4,5-dihydro-1,3,5-triazin-2-amine; and

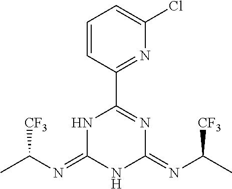 [SMILES: C[C@@H](/N=c1\nc(-c2cccc(Cl)n2)[nH]/c(=N\[C@H](C)C(F)(F)F)[nH]1)C(F)(F)F]

6-(6-chloropyridin-2-yl)-N2,N4-bis((R)-1,1,1-trifluoropropan-2-yl)-1,3,5-triazine-2,4(1H,3H)-diimine.

As used herein, the tautomers include the specified compounds, as well as any double bond isomers thereof.

In one embodiment, the disclosure relates to a compound that is:

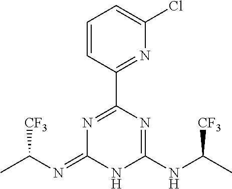 [SMILES: C[C@@H](/N=c1\nc(-c2cccc(Cl)n2)nc(N[C@H](C)C(F)(F)F)[nH]1)C(F)(F)F]

4-(6-chloropyridin-2-yl)-N—((R)-1,1,1-trifluoropropan-2-yl)-6-(((R)-1,1,1-trifluoropropan-2-yl)imino)-1,6-dihydro-1,3,5-triazin-2-amine;
or a pharmaceutically acceptable salt thereof.

In one embodiment, the disclosure relates to a compound that is:

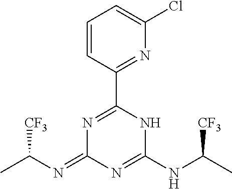 [SMILES: C[C@@H](/N=c1/nc(N[C@H](C)C(F)(F)F)[nH]c(-c2cccc(Cl)n2)n1)C(F)(F)F]

6-(6-chloropyridin-2-yl)-N—((R)-1,1,1-trifluoropropan-2-yl)-4-(((R)-1,1,1-trifluoropropan-2-yl)imino)-1,4-dihydro-1,3,5-triazin-2-amine;
or a pharmaceutically acceptable salt thereof.

In one embodiment, the disclosure relates to a compound that is:

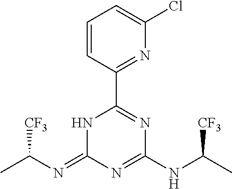 [SMILES: C[C@@H](/N=c1/nc(N[C@H](C)C(F)(F)F)nc(-c2cccc(Cl)n2)[nH]1)C(F)(F)F]

6-(6-chloropyridin-2-yl)-N—((R)-1,1,1-trifluoropropan-2-yl)-4-(((R)-1,1,1-trifluoropropan-2-yl)imino)-4,5-dihydro-1,3,5-triazin-2-amine;
or a pharmaceutically acceptable salt thereof.

In one embodiment, the disclosure relates to a compound that is:

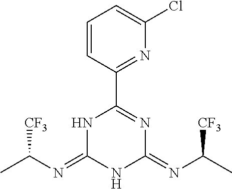 [SMILES: C[C@@H](/N=c1\nc(-c2cccc(Cl)n2)[nH]/c(=N\[C@H](C)C(F)(F)F)[nH]1)C(F)(F)F]

6-(6-chloropyridin-2-yl)-N2,N4-bis((R)-1,1,1-trifluoropropan-2-yl)-1,3,5-triazine-2,4(1H,3H)-diimine;
or a pharmaceutically acceptable salt thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” of a compound includes any non-toxic salt that, upon administration to a recipient, is capable of providing, either directly or indirectly, the compound. Pharmaceutically acceptable salts are described in detail in S. M. Berge, et al., J. Pharmaceutical Sciences, 1977, 66, 1-19, incorporated herein by reference.

### Methods of Preparing Solid Forms of the Compound of Formula (I)

The disclosure also relates to methods of preparing solid forms of the compound of formula (I).

In one aspect, the disclosure relates to a process for the preparation of a pharmaceutically acceptable salt of the compound of formula (I), comprising
dissolving the compound of formula (I) and a pharmaceutically acceptable acid in a solvent to afford a solution; and
precipitating the corresponding salt.

In one aspect, the disclosure relates to a process for the preparation of a crystalline form of a pharmaceutically acceptable salt of the compound of formula (I), comprising
dissolving the compound of formula (I) and a pharmaceutically acceptable acid in a solvent to afford a solution; and
precipitating the corresponding salt in a crystalline form.

In some embodiments, the solution is seeded in order to obtain a crystalline pharmaceutically acceptable salt.

In some embodiments, the pharmaceutically acceptable acid used in the process for the preparation of a pharmaceutically acceptable salt of the compound of formula (I) is selected from the group consisting of benzene sulfonic acid, (+)-camphor-10-sulfonic acid, ethane-1,2-disulfonic acid, ethanesulfonic acid, hydrobromic acid, hydrochloric acid, naphthalene-1,5-disulfonic acid, naphthalene-2-sulfonic acid, p-toluenesulfonic acid sulfuric acid and ortho phosphoric acid.

The pharmaceutically acceptable salt employed in the process may be crystalline or amorphous and may be in any state of hydration or solvation.

The solvent employed in the process may be any liquid or mixture of liquids suitable to dissolve the compound of formula (I) and the pharmaceutically acceptable salt. In some embodiments, the solvent comprises a polar organic solvent, such as methanol, ethyl acetate, acetonitrile, acetone, tetrahydrofurane (THF), or n-butanol that may be mixed with water. In some embodiments, the solvent comprises acetone and water. In some embodiments, the solvent comprises acetone and water (3:1 v/v to 1:/1 v/v).

The compound of formula (I) and the pharmaceutically acceptable acid may be dissolved in the solvent in any molar ratio and in any concentration that allows for subsequent precipitation of the cocrystal from the solution. In some embodiments, the compound of formula (I) and pharmaceutically acceptable acid are contacted with the solvent in a molar ratio of between about 1:2 and 2:1, or a molar ratio of about 2:1, 1:1 or 1:2. In some embodiments, the amount of the compound of formula (I) contacted with the solvent is sufficient to form about a 0.1 M to 0.4 M solution, based on the amount of the compound of formula (I). As a person of ordinary skill in the art would understand, however, in the event that some of the compound of formula (I) and/or pharmaceutically acceptable acid does not dissolve in the solvent, the actual molar ratio of pharmaceutically acceptable acid and the compound of formula (I) in solution, and the actual concentration of the compound of formula (I) the solution, may differ from that which would be calculated from the amounts of the compound of formula (I) and pharmaceutically acceptable acid contacted with the solvent.

In one aspect, the disclosure relates to a process for the preparation of a cocrystal of the compound of formula (I), comprising
dissolving the compound of formula (I) and a coformer in a solvent to afford a solution; and
precipitating the corresponding cocrystal.

In some embodiments, the solution is seeded.

In some embodiments, the coformer used in the method of preparing a cocrystal of the compound of formula (I) is selected from the group consisting of 3-hydroxy-2-naphtoic acid, L-serine, glycine, D-gluconic acid, glycolic acid, L-malic acid, oxalic acid, benzoic acid, fumaric acid, gentisic acid, glutaric acid, 4-hydroxybenzoic acid, alpha-ketoglutaric acid, malonic acid, salicylic acid, L-tartaric acid, urea, pyroglutamic acid, caproic acid, glycerol, L-lysine, S-proline and pyruvic acid.

The coformer employed in the process may be crystalline or amorphous and may be in any state of hydration or solvation.

The solvent employed in the process may be any liquid or mixture of liquids suitable to dissolve the compound of formula (I) and the coformer. In some embodiments, the solvent comprises a polar organic solvent, such as methanol, ethyl acetate, acetonitrile, acetone, tetrahydrofurane (THF), or n-butanol. In some embodiments, the solvent comprises acetone and water. In some embodiments, the solvent comprises acetone and water (4:1/1 to 2:1 v/v). In some embodiments, the solvent comprises ethyl acetate or methanol.

The compound of formula (I) and the coformer may be dissolved in the solvent in any molar ratio and in any concentration that allows for subsequent precipitation of the cocrystal from the solution. In some embodiments, the compound of formula (I) and coformer are contacted with the solvent in a molar ratio of between about 1:2 and 2:1, or a molar ratio of about 2:1, 1:1 or 1:2. In some embodiments, the amount of the compound of formula (I) contacted with the solvent is sufficient to form about a 0.1 M to 1 M solution, based on the amount of the compound of formula (I). As a person of ordinary skill in the art would understand, however, in the event that some of the compound of formula (I) and/or coformer does not dissolve in the solvent, the actual molar ratio of pharmaceutically acceptable acid and the compound of formula (I) in solution, and the actual concentration of the compound of formula (I) the solution, may differ from that which would be calculated from the amounts of the compound of formula (I) and coformer contacted with the solvent.

In another aspect, the disclosure relates to a process for the preparation of an amorphous solid dispersion of the compound of formula (I).

In some embodiments, the method comprises spray-drying a mixture comprising the compound of formula (I), a polymer, and an appropriate solvent or solvent mixture.

In some embodiments, the solvent is a volatile solvent (e.g., methylene chloride, acetone, methanol, ethanol, chloroform, tetrahydrofuran (THF), or a mixture thereof). In some embodiments, the solvent is acetone.

In some embodiments, the compound of formula (I) used in the spray-drying procedure is in the form of a pharmaceutically acceptable salt, or a crystalline form thereof, or a cocrystal, or a crystalline form thereof, in accordance with any of the embodiments described herein.

Spray drying involves atomization of a liquid mixture containing, e.g., a solid and a solvent or solvent mixture, and removal of the solvent or solvent mixture. Atomization may be done, for example, through a two-fluid or pressure or electrosonic nozzle or on a rotating disk.

Removal of the solvent or solvent mixture may require a subsequent drying step, such as tray drying, fluid bed drying (e.g., from about room temperature to about 100° C.), vacuum drying, microwave drying, rotary drum drying or biconical vacuum drying (e.g., from about room temperature to about 200° C.). Techniques and methods for spray-drying may be found in Perry's Chemical Engineering Handbook, 6th Ed., R. H. Perry, D. W. Green & J. O.

Maloney, eds., McGraw-Hill Book Co. (1984); and Marshall “Atomization and Spray-Drying” 50, Chem. Eng. Prog. Monogr. Series 2 (1954).

As used herein, the term “dissolving,” when referring to dissolving one or more substances in a solvent to afford a solution, means contacting the substance(s) with an amount of solvent sufficient to dissolve at least some of each of the substance(s). The mixture comprising the substance(s) and solvent may be stirred and/or warmed to facilitate the dissolution of the substance(s) in the solvent. As a person of ordinary skill in the art would understand, some undissolved material (including some of the substance(s) and/or some other material) may remain suspended in the solution, and such suspended material may be separated from the solution (e.g., by filtration or decantation) prior to precipitation of a solid form. In some embodiments, water is added to the solution prior to precipitation of a solid form.

As used herein, the term “about,” when referring to a molar ratio or concentration (e.g., molarity), means that the molar ratio or concentration has the specified value+10%. For example, a molar ratio of “about 2:1” would include molar ratios between 1.8:1 and 2.2:1. Similarly, a concentration of “about 1.5 M” would include concentrations between 1.35 M and 1.65 M.

As used herein, the term “precipitating,” when referring to precipitating a solid form from a solution, means causing the solid form to precipitate from the solution. Without intending to be bound by any theory, precipitation may be caused by saturating the solution with the solid form (e.g., by increasing the concentration of the solid form in the solution or by reducing the solubility of the solid form in the solution).

In some embodiments, “precipitating” comprises cooling the solution. Without intending to be bound by any theory, cooling the solution may cause precipitation of the solid form by decreasing the solubility of the solid form in the solution, such that the solid form reaches its saturation concentration.

In some embodiments, “precipitating” comprises evaporating a portion of the solvent from the solution. Without intending to be bound by any theory, evaporating solvent from the solution may cause precipitation of the solid form by increasing the concentration of the solid form in the solution to its saturation concentration.

In some embodiments, “precipitating” comprises adding an antisolvent to the solution. As used herein, the term “antisolvent” refers to a liquid in which the solid form is less soluble than the solvent used to form the solution. Without intending to be bound by any theory, the addition of an antisolvent to the solution may cause precipitation of the solid form by decreasing the solubility of the solid form in the solution, such that the solid form reaches its saturation concentration. In some embodiments, the antisolvent comprises a non-polar organic solvent. In some embodiments, the antisolvent comprises toluene. In some embodiments, the antisolvent comprises methyl tert-butyl ether. In some embodiments, the antisolvent comprises a C₅-C₁₂ alkane or cycloalkane.

In some embodiments, “precipitating” comprises seeding the solution with crystals of the solid form to be precipitated from solution. As used herein, the term “seeding” refers to the addition of a particular crystalline material to a solution to initiate recrystallization or crystallization of that particular crystalline material.

As used herein, the term “C₅-C₁₂ alkane or cycloalkane” means a saturated straight-chain, branched, or cyclic hydrocarbon having five to twelve carbon atoms. Examples include pentane, hexane, heptane, octane, cyclohexane, and the like.

In some embodiments, the method further comprises isolating the solid form. As used herein, the term “isolating” means separating the precipitated solid form from the solution. Such separation may be accomplished by any means known in the art, including without limitation filtration of the precipitated solid form and decantation of the solution from the precipitated solid form.

### Compositions and Routes of Administration

In another aspect, the disclosure relates to a pharmaceutical composition comprising a solid form, i.e. a pharmaceutically acceptable salt or cocrystal of compound of formula (I), as described in any of the embodiments herein, and one or more pharmaceutical excipients.

In another aspect, the disclosure relates to a pharmaceutical composition comprising a therapeutically effective amount of a solid form, i.e. a pharmaceutically acceptable salt or cocrystal of compound of formula (I), as described in any of the embodiments herein, and one or more pharmaceutical excipients.

As used herein, the term “therapeutically effective amount,” when referring to an amount of the solid form, i.e. pharmaceutically acceptable salt or cocrystal described herein, refers to an amount that will elicit a biological or medical response in a patient, such as reducing or inhibiting an enzyme or a protein activity, alleviating or ameliorating certain symptoms, curing a disease, lessening the severity of a disease, slowing or delaying the progression of a disease, or preventing a disease. In some embodiments, the term “therapeutically effective amount” refers to the amount of solid form, i.e. pharmaceutically acceptable salt or cocrystal described herein that, when administered to a patient, is effective to inhibit mutant IDH1 and/or mutant IDH2. In other embodiments, the term “therapeutically effective amount” refers to the amount of the solid form, i.e. pharmaceutically acceptable salt or cocrystal described herein that, when administered to a patient, is effective to treat a cancer in the patient.

As used herein, the term “pharmaceutical excipient” refers to a carrier, adjuvant, or vehicle that may be administered to a patient together with the solid form, i.e. pharmaceutically acceptable salt or cocrystal described herein, that does not destroy the pharmacological activity of the compound of formula (I), and that is nontoxic when administered in doses sufficient to deliver a therapeutic amount of the compound of formula (I).

Pharmaceutical excipients that may be used in the pharmaceutical compositions described herein include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the compound of formula (I).

In some cases, the pH of the pharmaceutical composition may be adjusted with pharmaceutically acceptable acids, bases or buffers.

The pharmaceutical compositions described herein may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically acceptable excipients.

As used herein, “parenteral” administration includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, excipients which are commonly used include lactose, corn starch, microcrystalline cellulose, croscarmellose sodium, hydroxypropyl cellulose, colloidal silicon dioxide, and sodium lauryl sulfate.

Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing the solid form, i.e. pharmaceutically acceptable salt or cocrystal described herein, with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a pharmaceutically acceptable excipient suitable for topical administration, including without limitation mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this invention may also be topically applied to the lower intestinal tract by rectal suppository formulation or in a suitable enema formulation. Transdermal patches are also included in one aspect of this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The amount of active ingredient that may be combined with one or more pharmaceutical excipients to produce a single dosage form will vary depending upon the patient treated and the particular mode of administration. A typical preparation will contain from about 5% to about 95% active compound (w/w). Alternatively, such preparations contain from about 20% to about 80% active compound. In some embodiments, the pharmaceutical composition comprises 1-10% w/w of the compound of formula (I) (based on the weight of the free compound of formula (I), apart from the weight of any coformer, salt former, water of hydration, solvent of solvation, and the like). In some embodiments, the pharmaceutical composition comprises 20-30% w/w of the compound of formula (I) (based on the weight of the free compound of formula (I), apart from the weight of any coformer, salt former, water of hydration, solvent of solvation, and the like). In some embodiments. The pharmaceutical composition comprises about 10 mg, about 25 mg, about 40 mg, about 50 mg, about 100 mg, about 200 mg, or about 300 mg of the compound of formula (I) (based on the weight of the free compound of formula (I), apart from the weight of any coformer, salt former, water of hydration, solvent of solvation, and the like). In some embodiments, the pharmaceutical composition comprises about 10 mg or about 40 mg of the compound of formula (I) (based on the weight of the free compound of formula (I), apart from the weight of any coformer, salt former, water of hydration, solvent of solvation, and the like).

The pharmaceutical compositions may further comprise a therapeutically effective amount of an additional therapeutic agent, including without limitation any one of the additional therapeutic agents identified below as being useful in combination therapy.

As used herein, the term “therapeutically effective amount,” when referring to an amount of an additional therapeutic agent, refers to an amount of the agent that will elicit a biological or medical response in a patient, such as reducing or inhibiting an enzyme or a protein activity, alleviating or ameliorating certain symptoms, curing a disease, lessening the severity of a disease, slowing or delaying the progression of a disease, or preventing a disease.

In another aspect, the invention relates to a pharmaceutical composition prepared by a process comprising mixing a therapeutically effective amount of a solid form, i.e. pharmaceutically acceptable salt or cocrystal, as described in any of the embodiments herein, with one or more pharmaceutical excipients to afford the pharmaceutical composition.

As used here, the term “mixing” means includes any process in which the solid form, i.e. pharmaceutically acceptable salt or cocrystal described herein is contacted with one or more pharmaceutical excipients to afford a pharmaceutical composition, regardless of whether the pharmaceutical composition so obtained contains the solid form, i.e. pharmaceutically acceptable salt or cocrystal described herein. Thus, the term “mixing” includes processes in which the solid form, i.e. pharmaceutically acceptable salt or cocrystal described herein remains in the same solid form, i.e. pharmaceutically acceptable salt or cocrystal described herein is dissolved and/or converted to a different solid form. Examples of “mixing” processes including wet or dry blending, wet or dry granulation, suspension of the solid form, i.e. pharmaceutically acceptable salt or cocrystal described herein in the pharmaceutical excipient, and the like.

Uses of Solid Forms, i.e. Pharmaceutically Acceptable Salts or Cocrystals Described Herein and Pharmaceutical Compositions Thereof

In another aspect, the invention relates to a method of treating a cancer characterized by the presence of an IDH1 and/or IDH2 mutation in a patient in need thereof, comprising administering a therapeutically effective amount of a solid form, i.e. pharmaceutically acceptable salt or cocrystal, or a pharmaceutical composition thereof, as described in any of the embodiments herein, to the patient.

In another aspect, the invention relates to the use of a solid form, i.e. pharmaceutically acceptable salt or cocrystal, or a pharmaceutical composition thereof, as described in any of the embodiments herein, for the manufacture of a medicament for use in treating a cancer characterized by the presence of an IDH1 and/or IDH2 mutation in a patient in need thereof. In another aspect, the invention relates to a solid form, i.e. pharmaceutically acceptable salt or cocrystal, or a pharmaceutical composition thereof, as described in any of the embodiments herein, for use in treating a cancer characterized by the presence of an IDH1 and/or IDH2 mutation in a patient in need thereof.

As used herein, the terms “treat” and “treating,” when referring to a cancer, mean having a therapeutic effect on, alleviating or ameliorating one or more symptoms of, altering the progression of, eradicating, reducing the size of, slowing or inhibiting the growth or progression of, delaying or minimizing one or more symptoms associated with, reducing the malignancy of, or inducing stasis of the cancer. When referring to a disease other than a cancer, the terms “treat” and “treating” mean having a therapeutic effect on, alleviating or ameliorating one or more symptoms of, altering the progression of, eradicating, or delaying or minimizing one or more symptoms associated with the disease.

As used herein, the term “patient” refers to a mammal, including mice, rats, dogs and humans, which is afflicted with a cancer characterized by the presence of an IDH1 and/or IDH2 mutation. In some embodiments, the patient is a human. In some embodiments, the patient is a human adult (i.e., a human at least 18 years of age). In some embodiments, the patient is a human child (i.e., a human under 18 years of age).

In some embodiments, the cancer is characterized by the presence of an IDH1 mutation. In other embodiments, the IDH1 mutation is an R132X mutation. In other embodiments, the IDH1 mutation is an R132H or R132C mutation. In other embodiments, the IDH1 mutation is an R132H, R132C, R132L, R132V, R132S, or R132G mutation. In other embodiments, the IDH1 mutation is an R132H mutation. In other embodiments, the IDH1 mutation is an R132C mutation. In other embodiments, the IDH1 mutation is an R132S, R132L or R132G mutation. In other embodiments, the IDH1 mutation results in accumulation of R(−)-2-hydroxyglutarate in the patient. In other embodiments, the IDH1 mutation results in a new ability of IDH1 to catalyze the NADPH-dependent reduction of α-ketoglutarate to R(−)-2-hydroxyglutarate.

Thus, in some embodiments, treating a cancer characterized by an IDH1 mutation comprises inhibiting mutant IDH1 activity.

In some embodiments, the cancer is a tumor wherein at least 30, 40, 50, 60, 70, 80 or 90% of the tumor cells carry an IDH1 mutation, and in particular an IDH1 R132H, R132C, R132S, R132L or R132G mutation, at the time of diagnosis or treatment.

Without being bound by theory, applicants believe that mutant alleles of IDH1 wherein the IDH1 mutation results in a new ability of the enzyme to catalyze the NADPH-dependent reduction of α-ketoglutarate to R(−)-2-hydroxyglutarate, and in particular R132H, R132C, R132S, R132L or R132G mutations of IDH1, characterize a subset of all types of cancers, without regard to their cellular nature or location in the body. Thus, the compounds and methods of this invention are useful to treat any type of cancer that is characterized by the presence of a mutant allele of IDH1 imparting such activity, and in particular IDH1 R132H, R132C, R132S, R132L or R132G mutations.

As shown in Table 1, IDH1 R132X mutations are known to occur in a variety of cancers.

TABLE 1
Cancers Associated with IDH1 R132X Mutations

| | IDH1 R132X | |
|---|---|---|
| Cancer Type | Mutation | Tumor Type |
| brain tumors | R132H | primary tumor |
| | R132C | primary tumor |
| | R132S | primary tumor |
| | R132G | primary tumor |
| | R132L | primary tumor |
| | R132V | primary tumor |
| fibrosarcoma | R132C | HT1080 fibrosarcoma |
| | | cell line |
| Acute Myeloid Leukemia | R132H | primary tumor |
| (AML) | R132G | primary tumor |
| | R132C | primary tumor |
| Prostate cancer | R132H | primary tumor |
| | R132C | primary tumor |
| Acute lymphoblastic | R132C | primary tumor |
| leukemia (ALL) | | |
| paragangliomas | R132C | primary tumor |

Accordingly, in some embodiments, the cancer is a cancer selected from the cancer types listed in Table 1, and the IDH1 mutation is one or more of the IDH1 R132X mutations listed in Table 1 for that particular cancer type.

IDH1 R132H mutations have been identified in glioma, acute myelogenous leukemia, sarcoma, melanoma, non-small cell lung cancer, cholangiocarcinomas, chondrosarcoma, myelodysplastic syndromes (MDS), myeloproliferative neoplasm (MPN), colon cancer, and angio-immunoblastic non-Hodgkin's lymphoma (NHL). Accordingly, in some embodiments, the cancer is selected from glioma, acute myelogenous leukemia, sarcoma, melanoma, non-small cell lung cancer (NSCLC), cholangiocarcinomas, chondrosarcoma, myelodysplastic syndromes (MDS), myeloproliferative neoplasm (MPN), colon cancer, or angio-immunoblastic non-Hodgkin's lymphoma (NHL). In some embodiments, the cancer is glioma, and the glioma is a low grade glioma or a secondary high grade glioma. In other embodiments, the cancer is glioma, and the glioma is a low grade glioma (grade II), anaplastic (grade III) or glioblastoma (GBM, grade IV).

In some embodiments, the cancer is characterized by the presence of an IDH2 mutation. In other embodiments, the IDH2 mutation is an R140X mutation. In other embodiments, the IDH2 mutation is an R140Q, R140W, or R140L mutation. In other embodiments, the IDH2 mutation is an R172X mutation. In other embodiments, the IDH2 mutation is an R172K or R172G mutation. In other embodiments, the IDH2 mutation is an R140X mutation. In other embodiments, the IDH2 mutation is an R140Q mutation. In other embodiments, the IDH2 mutation is an R140W mutation. In other embodiments, the IDH2 mutation is an R140L mutation. In other embodiments, the IDH2 mutation is an R172X mutation. In other embodiments, the IDH2 mutation is an R172K mutation. In other embodiments, the IDH2 mutation is an R172G mutation. In other embodiments, the IDH2 mutation results in accumulation of R(−)-2-hydroxyglutarate in the patient. In other embodiments, the IDH2 mutation results in a new ability of IDH2 to catalyze the NADPH-dependent reduction of α-ketoglutarate to R(−)-2-hydroxyglutarate. Thus, in some embodiments, treating a cancer characterized by an IDH2 mutation comprises inhibiting mutant IDH2 activity.

Without being bound by theory, applicants believe that mutant alleles of IDH2 wherein the IDH2 mutation results in a new ability of the enzyme to catalyze the NADPH-dependent reduction of α-ketoglutarate to R(−)-2-hydroxyglutarate, and in particular R140Q and/or R172K mutations of IDH2, characterize a subset of all types of cancers, without regard to their cellular nature or location in the body. Thus, the compounds and methods of one aspect of this invention are useful to treat any type of cancer that is characterized by the presence of a mutant allele of IDH2 imparting such activity and in particular an IDH2 R140Q and/or R172K mutation.

In some embodiments, the cancer is a tumor wherein at least 30, 40, 50, 60, 70, 80 or 90% of the tumor cells carry an IDH2 mutation, and in particular an IDH2 R140Q, R140W, or R140L and/or R172K or R172G mutation, at the time of diagnosis or treatment.

In some embodiments, the cancer is characterized by the presence of an IDH1 mutation and an IDH2 mutation, wherein the IDH1 and IDH2 mutations collectively result in accumulation of R(−)-2-hydroxyglutarate in a patient.

In some embodiments, the brain tumor (e.g., glioma) is characterized by the presence of an IDH1 mutation and an IDH2 mutation, wherein the IDH1 and IDH2 mutations collectively result in accumulation of R(−)-2-hydroxyglutarate in a patient.

A cancer can be analyzed by sequencing cell samples to determine the presence and specific nature of any mutation(s) characterizing the cancer.

In some embodiments, the cancer is glioma, acute myelogenous leukemia, sarcoma, melanoma, non-small cell lung cancer (NSCLC), cholangiocarcinomas (e.g., intrahepatic cholangiocarcinoma (IHCC)), chondrosarcoma, myelodysplastic syndromes (MDS), myeloproliferative neoplasm (MPN), prostate cancer, chronic myelomonocytic leukemia (CMML), B-acute lymphoblastic leukemias (B-ALL), B-acute lymphoblastic leukemias (B-ALL), myeloid sarcoma, multiple myeloma, lymphoma colon cancer, or angio-immunoblastic non-Hodgkin's lymphoma (NHL). In some embodiments, the cancer is glioma, and the glioma is a low grade glioma or a secondary high grade glioma. In other embodiments, the cancer is glioma, the glioma is a low grade glioma (grade II), anaplastic (grade III) or glioblastoma (GBM, grade IV).

In some embodiments, the cancer is lymphoma (e.g., Non-Hodgkin lymphoma (NHL) such B-cell lymphoma (e.g., Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell lymphoma (e.g., mycosis fungoides, anaplastic large cell lymphoma, or precursor T-lymphoblastic lymphoma)).

In some embodiments, the cancer is glioma, myelodysplastic syndrome (MDS), myeloproliferative neoplasm (MPN), acute myelogenous leukemia (AML), sarcoma, melanoma, non-small cell lung cancer, chondrosarcoma, cholangiocarcinomas or angio-immunoblastic lymphoma. In other embodiments, the cancer is glioma, myelodysplastic syndrome (MDS), myeloproliferative neoplasm (MPN), acute myelogenous leukemia (AML), melanoma, chondrosarcoma, or angioimmunoblastic non-Hodgkin's lymphoma (NHL). In some embodiments, the cancer is glioma, and the glioma is a low grade glioma or a secondary high grade glioma. In other embodiments, the cancer is glioma, and the glioma is a low grade glioma (grade II), anaplastic (grade III) or glioblastoma (GBM, grade IV).

In some embodiments the cancer is refractory or relapsed. In other embodiments the cancer is newly diagnosed or previously untreated.

In one aspect of this embodiment, the efficacy of cancer treatment is monitored by measuring the levels of 2HG as described herein.

In some embodiments, the efficacy of cancer treatment is monitored by measuring the levels of 2HG in the patient. Typically levels of 2HG are measured prior to treatment, wherein an elevated level is indicated for the use of the compound of formula (I), including in the form of the solid form, i.e. pharmaceutically acceptable salt or pharmaceutical composition thereof, as described in any of the embodiments herein, to treat the cancer. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy.

Similarly, a determination that 2HG levels are not elevated during the course of or following treatment is also indicative of efficacy. Typically, 2HG measurements will be utilized together with other well-known determinations of efficacy of cancer treatment, such as reduction in number and size of tumors and/or other cancer-associated lesions, improvement in the general health of the patient, and alterations in other biomarkers that are associated with cancer treatment efficacy.

2HG can be detected in a sample by LC/MS. The sample is mixed 80:20 with methanol, and centrifuged at 3,000 rpm for 20 minutes at 4 degrees Celsius. The resulting supernatant can be collected and stored at −80 degrees Celsius prior to LC-MS/MS to assess 2-hydroxyglutarate levels. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions.

Metabolites can be separated by reversed phase chromatography using 10 mM tributyl-amine as an ion pairing agent in the aqueous mobile phase, according to a variant of a previously reported method (Luo et al. J Chromatogr A 1147, 153-64, 2007). One method allows resolution of TCA metabolites: t=0, 50% B; t=5, 95% B; t=7, 95% B; t=8, 0% B, where B refers to an organic mobile phase of 100% methanol. Another method is specific for 2-hydroxyglutarate, running a fast linear gradient from 50%-95% B (buffers as defined above) over 5 minutes. A Synergi Hydro-RP, 100 mm×2 mm, 2.1 μm particle size (Phenomonex) can be used as the column, as described above. Metabolites can be quantified by comparison of peak areas with pure metabolite standards at known concentration. Metabolite flux studies from 13C-glutamine can be performed as described, e.g., in Munger et al. Nat Biotechnol 26, 1179-86, 2008.

In some embodiments, 2HG is directly evaluated.

In other embodiments, a derivative of 2HG formed in the process of performing the analytic method is evaluated. By way of example, such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis.

In another embodiment a metabolic derivative of 2HG is evaluated. Examples include species that build up or are elevated, or reduced, as a result of the presence of 2HG, such as glutarate or glutamate that will be correlated to 2HG, e.g., R-2HG.

Exemplary 2HG derivatives include dehydrated derivatives such as the compounds provided below or a salt adduct thereof:

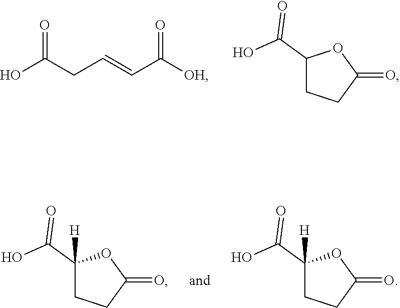 [SMILES: O=C(O)/C=C/CC(=O)O ; O=C1CCC(C(=O)O)O1 ; O=C1CC[C@H](C(=O)O)O1 ; O=C1CC[C@H](C(=O)O)O1]

In some embodiments, various evaluation steps are performed prior to and/or following treatment of a cancer with the solid form, i.e. pharmaceutically acceptable salt or cocrystal described herein or pharmaceutical composition thereof. Thus, in some embodiments, the method described herein further comprises an evaluation step prior to and/or after treatment with the solid form, i.e. pharmaceutically acceptable salt or cocrystal described herein or pharmaceutical composition thereof.

In some embodiments, the evaluation steps comprise evaluating the growth, size, weight, invasiveness, stage and/or other phenotype of the cancer. Thus, in some embodiments, the method described herein further comprises the step of evaluating the growth, size, weight, invasiveness, stage and/or other phenotype of the cancer prior to and/or after treatment with the solid form, i.e. pharmaceutically acceptable salt or cocrystal described herein or pharmaceutical composition thereof.

In some embodiments, prior to and/or after treatment with the solid form, i.e. pharmaceutically acceptable salt or cocrystal described herein or pharmaceutical composition thereof, the method further comprises the step of evaluating the IDH1 genotype of the cancer. This may be achieved by ordinary methods in the art, such as DNA sequencing, immuno analysis, and/or evaluation of the presence, distribution or level of 2HG.

In some embodiments, prior to and/or after treatment with the solid form, i.e. pharmaceutically acceptable salt or cocrystal described herein or pharmaceutical composition thereof, the method further comprises the step of determining the 2HG level in the patient. This may be achieved by spectroscopic analysis, e.g., magnetic resonance-based analysis, e.g., MRI and/or MRS measurement, sample analysis of bodily fluid, such as serum or spinal cord fluid analysis, or by analysis of surgical material, e.g., by mass-spectroscopy.

2HG is known to accumulate in the inherited metabolic disorder 2-hydroxyglutaric aciduria. This disease is caused by deficiency in the enzyme 2-hydroxyglutarate dehydrogenase, which converts 2HG to α-KG (Struys, E. A. et al. Am J Hum Genet 76, 358-60 (2005)). Patients with 2-hydroxyglutarate dehydrogenase deficiencies accumulate 2HG in the brain as assessed by MRI and CSF analysis, develop leukoencephalopathy, and have an increased risk of developing brain tumors (Aghili, M., Zahedi, F. & Rafiee, J Neurooncol 91, 233-6 (2009); Kolker, S., Mayatepek, E. & Hoffmann, G. F. Neuropediatrics 33, 225-31 (2002); Wajner, M., Latini, A., Wyse, A. T. & Dutra-Filho, C. S. J Inherit Metab Dis 27, 427-48 (2004)). Furthermore, elevated brain levels of 2HG result in increased ROS levels (Kolker, S. et al. Eur J Neurosci 16, 21-8 (2002); Latini, A. et al. Eur J Neurosci 17, 2017-22 (2003)), potentially contributing to an increased risk of cancer. The ability of 2HG to act as an NMDA receptor agonist may contribute to this effect (Kolker, S. et al. Eur J Neurosci 16, 21-8 (2002)). 2HG may also be toxic to cells by competitively inhibiting glutamate and/or aKG utilizing enzymes. These include transaminases which allow utilization of glutamate nitrogen for amino and nucleic acid biosynthesis, and aKG-dependent prolyl hydroxylases such as those which regulate HIF1-alpha levels.

Thus, according to another embodiment, one aspect of the invention provides a method of treating 2-hydroxyglutaric aciduria, particularly D-2-hydroxyglutaric aciduria, in a patient by administering to the patient a therapeutically effective amount of the solid form, i.e. pharmaceutically acceptable salt or cocrystal, or a pharmaceutical composition thereof, as described in any one of the embodiments herein.

Also provided are methods of treating a disease selected from Maffucci syndrome and Ollier disease, characterized by the presence of a mutant allele of IDH1 comprising the step of administering to patient in need thereof a therapeutically effective amount of the solid form, i.e. pharmaceutically acceptable salt or cocrystal, or a pharmaceutical composition thereof, as described in any one of the embodiments herein.

Treatment methods described herein can additionally comprise various evaluation steps prior to and/or following treatment with the solid form, i.e. pharmaceutically acceptable salt or cocrystal described herein or pharmaceutical composition thereof.

In one embodiment, prior to and/or after treatment with the solid form, i.e. pharmaceutically acceptable salt or cocrystal described herein or pharmaceutical composition thereof, the method further comprises the step of evaluating the growth, size, weight, invasiveness, stage and/or other phenotype of the cancer.

In one embodiment, prior to and/or after treatment with the solid form, i.e. pharmaceutically acceptable salt or cocrystal described herein or pharmaceutical composition thereof, the method further comprises the step of evaluating the IDH2 genotype of the cancer. This may be achieved by ordinary methods in the art, such as DNA sequencing, immuno analysis, and/or evaluation of the presence, distribution or level of 2HG.

All the dosages disclosed herein are based on the amount of the compound of formula (I), that is to say on the amount of compound of formula (I) free base contained in the administered pharmaceutical composition.

The solid form, i.e. pharmaceutically acceptable salt or cocrystal, and pharmaceutical compositions thereof, as described in any of the embodiments herein, can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between about 1 mg and about 1000 mg/dose, every 4 to 120 hours, based on the amount of the compound of formula (I). In some embodiments, the solid form, i.e. pharmaceutically acceptable salt or cocrystal described herein or pharmaceutical composition thereof is administered once, twice, or three times a day. In other embodiments, the solid form, i.e. pharmaceutically acceptable salt or cocrystal described herein or pharmaceutical composition thereof is administered once a day. In other embodiments, the solid form, i.e. pharmaceutically acceptable salt or cocrystal described herein, or pharmaceutical composition thereof is administered twice a day. In other embodiments, the solid form, i.e. pharmaceutically acceptable salt or cocrystal described herein, or pharmaceutical composition thereof is administered three times a day. The methods herein contemplate administration of a therapeutically effective amount of the solid form, i.e. pharmaceutically acceptable salt or cocrystal described herein or pharmaceutical composition thereof so as to achieve the desired or stated effect. Typically, the pharmaceutical compositions of one aspect of this invention will be administered from about 1 to about 6 times per day or alternatively, as a continuous infusion. In some embodiments, the solid form, i.e. pharmaceutically acceptable salt or cocrystal described herein, or pharmaceutical composition thereof is administered once a day. In other embodiments, the solid form, i.e. pharmaceutically acceptable salt or cocrystal described herein, or pharmaceutical composition thereof is administered twice a day. Such administration can be used as a chronic or acute therapy.

In some embodiments, the solid form, i.e. pharmaceutically acceptable salt or cocrystal, or pharmaceutical composition thereof, as described in any of the embodiments herein, is administered in a dosage, based on the amount of the compound of formula (I), of: (1) from 1 to 100 mg/day, 2 to 50 mg/day, 3 to 30 mg/day, 4 to 20 mg/day, 5 to 15 mg/day, 8 to 12 mg/day, or about 10 mg/day; (2) from 1 to 500 mg/day, 1 to 250 mg/day, 5 to 100 mg/day, 8 to 75 mg/day, 10 to 50 mg/day, 15 to 40 mg/day, 20 to 30 mg/day, about 20 mg/day, or about 25 mg/day; (3) from 1 to 500 mg/day, 10 to 250 mg/day, 20 to 100 mg/day, 30 to 80 mg/day, 40 to 60 mg/day, 45 to 55 mg/day, about 40 mg/day or about 50 mg/day; (4) from 1 to 500 mg/day, 20 to 400 mg/day, 40 to 200 mg/day, 50 to 150 mg/day, 75 to 125 mg/day, 85 to 115 mg/day, 90 to 110 mg/day, or about 100 mg/day; (5) from 1 to 500 mg/day, 50 to 400 mg/day, 100 to 300 mg/day, 150 to 250 mg/day, 175 to 225 mg/day, 185 to 215 mg/day, 190 to 210 mg/day, or about 200 mg/day; or (6) from 1 to 500 mg/day, 100 to 500 mg/day, 200 to 400 mg/day, 250 to 350 mg/day, 275 to 375 mg/day, 285 to 315 mg/day, 290 to 310 mg/day, or about 300 mg/day.

In some embodiments, the solid form, i.e. pharmaceutically acceptable salt or cocrystal, or pharmaceutical composition thereof, as described in any of the embodiments herein, is administered in a dosage, based on the amount of the compound of formula (I), of from 0.01 to 10 mg/kg of body weight per day, 0.2 to 8.0 mg/kg of body weight per day, 0.4 to 6.0 mg/kg of body weight per day, 0.6 to 4.0 mg/kg of body weight per day, 0.8 to 2.0 mg/kg of body weight per day, 0.1 to 1 mg/kg of body weight per day, 0.2 to 1.0 mg/kg of body weight per day, 0.15 to 1.5 mg/kg of body weight per day, or 0.1 to 0.5 mg/kg of body weight per day. In some embodiments, the solid form, i.e. pharmaceutically acceptable salt or cocrystal, or pharmaceutical composition thereof, as described in any of the embodiments herein, is administered once per day or more than once per day (e.g., twice per day, three times per day, four times per day, etc.) to achieve administration of the daily dosages described herein.

In some embodiments, the solid form, i.e. pharmaceutically acceptable salt or cocrystal, or pharmaceutical composition thereof, as described in any of the embodiments herein, is administered once per day to achieve administration of the daily dosages described herein.

In some embodiments, the solid form, i.e. pharmaceutically acceptable salt or cocrystal, or pharmaceutical composition thereof, as described in any of the embodiments herein, is administered twice per day to achieve administration of the daily dosages described herein.

In some embodiments, the solid form, i.e. pharmaceutically acceptable salt or cocrystal, or pharmaceutical composition thereof, as described in any of the embodiments herein, is administered once per day in a dosage, based on the amount of the compound of formula (I), of: (1) about 10 mg, about 20 mg, about 25 mg, about 40 mg, about 50 mg, about 100 mg, about 200 mg, or about 300 mg per administration; (2) 30-70 mg, 35-65 mg, 40-60 mg, 45-55 mg, about 40 mg or about 50 mg per administration; or (3) 5-35 mg, 5-20 mg, 5-15 mg, about 20 mg or about 10 mg per administration. In some embodiments, the solid form, i.e. pharmaceutically acceptable salt or cocrystal, or pharmaceutical composition thereof, as described in any of the embodiments herein, is administered twice per day in a dosage, based on the amount of the compound of formula (I), of: (1) 30-70 mg, 35-65 mg, 40-60 mg, 45-55 mg, about 40 mg or about 50 mg per administration; or (2) 5-35 mg, 5-20 mg, 5-15 mg, about 20 mg or about 10 mg per administration. The amounts of the solid form, i.e. pharmaceutically acceptable salt or cocrystal, or pharmaceutical composition thereof, set forth herein are based on the amount of the compound of formula (I). Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

As used herein, the term “about,” when referring to a dosage, means that the dosage has the specified value ±10%. For example, a dosage of “about 100 mg/kg” would include dosages between 90 mg/kg and 110 mg/kg.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of the compound of formula (I), administered as the solid form, i.e. pharmaceutically acceptable salt or cocrystal, or a pharmaceutical composition thereof, as described in any of the embodiments herein, or combination of one aspect of this invention may be administered, if necessary.

Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Previously Treated Patients

In some embodiments, the patient in need of treatment for a cancer characterized by the presence of an IDH1 and/or IDH2 mutation was previously administered a cancer therapy.

In some embodiments, the patient was previously administered a cancer therapy for the cancer. The previously administered cancer therapy may have been effective or ineffective in treating the cancer or may have been effective for some period of time in treating the cancer.

As used herein, the term “cancer therapy” refers to a cancer therapeutic agent or a cancer treatment. As used herein, the term “cancer therapeutic agent” refers to a therapeutic agent (other than the compound of formula (I), the solid form, i.e. pharmaceutically acceptable salt or cocrystal described herein, or the pharmaceutical composition thereof) that is indicated for treating a cancer. Cancer therapeutic agents include, for example, chemotherapy, targeted therapy agents, antibody therapies, immunotherapy agents, hormonal therapy agents, and check point inhibitors. Examples of each of these classes of cancer therapeutic agents are provided below. As used herein, the term “cancer treatment” refers to a treatment that is indicated for treating a cancer. Cancer treatments include, for example, surgery and radiation therapy.

In some embodiments, the cancer therapeutic agent is a chemotherapy agent. Examples of chemotherapy agents used in cancer therapy include, for example, antimetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-aza-deoxycytidine), zebularine, isothiocyanates, azacitidine (5-azacytidine), 5-flouro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy.

In some embodiments, the cancer therapeutic agent is a differentiation agent. Differentiation agents include retinoids (such as all-trans-retinoic acid (ATRA), 9-cis retinoic acid, 13-cis-retinoic acid (13-cRA) and 4-hydroxy-phenretinamide (4-HPR)); arsenic trioxide; histone deacetylase inhibitors HDACs (such as azacytidine (Vidaza) and butyrates (e.g., sodium phenylbutyrate)); hybrid polar compounds (such as hexamethylene bisacetamide ((HMBA)); vitamin D; and cytokines (such as colony-stimulating factors including G-CSF and GM-CSF, and interferons).

In some embodiments, the cancer therapeutic agent is a targeted therapy agent. Targeted therapy constitutes the use of agents specific for the deregulated proteins of cancer cells.

Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. Other targeted therapy agents include biguanides such as metformin or phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, the cancer therapeutic agent is an antibody. Monoclonal antibody therapy is a strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox.

In some embodiments, the cancer therapeutic agent is an immunotherapy agent. Cancer immunotherapy refers to a diverse set of therapeutic strategies designed to induce the patient's own immune system to fight the tumor. Contemporary methods for generating an immune response against tumors include intravesicular BCG immunotherapy for superficial bladder cancer and use of interferons and other cytokines to induce an immune response in renal cell carcinoma and melanoma patients.

Allogeneic hematopoietic stem cell transplantation can be considered a form of immunotherapy, since the donor's immune cells will often attack the tumor in a graft-versus-tumor effect.

In some embodiments, the cancer therapeutic agent is a hormonal therapy agent. The growth of some cancers can be inhibited by providing or blocking certain hormones.

Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

In some embodiments, the cancer therapeutic agent is a check point inhibitor. Check point inhibitor therapy is a form of cancer treatment in which manipulation of immune system checkpoints is used restore immune system function against cancer cells. Examples of check point inhibitors include ipilimumab, nivolumab, pembrolizumab, atezolizumab, avelumab, durvalumab, and the like.

Other cancer therapeutic agents include imatinib, gene therapy, peptide and dendritic cell vaccines, synthetic chlorotoxins, radiolabeled drugs and antibodies, Chimeric antigen receptors or CAR-Ts (e.g., Kymriah® (tisagenlecleucel), Yescarta® (axicabtagene ciloleucel)), Gliadel® (carmustine implant), and Avastin® (bevacizumab).

In some embodiments, the cancer treatment is radiation therapy. Radiation therapy involves the use of high-energy radiation (e.g., x-rays, gamma rays, or charged particles) to damage and/or kill cancer cells and to shrink tumors. In the methods of the invention, radiation may be delivered to the brain tumor (e.g., glioma) by a machine positioned outside the body (external-beam radiation therapy), by radioactive material placed in the body near the brain tumor (internal radiation therapy, also called brachytherapy), or by radioactive substances administered systemically (e.g., radioactive iodine) that travel through the bloodstream to the brain tumor. Alternatively, these delivery methods can be used in combination.

In some embodiments, the radiation therapy comprises external radiation therapy (e.g., external-beam radiation therapy including fractionated external-beam radiation therapy, stereotactic radiation such as Cyberknife® or Gamma Knife®, proton therapy, and the like), where the radiation is delivered to the brain tumor (e.g., glioma) by an instrument outside the body. External radiation therapy may be given as a course of several treatments over days or weeks. In one aspect of these embodiments, the radiation is administered in the form of x-rays.

In other embodiments, the radiation therapy comprises internal radiation therapy, where the radiation comes from an implant or a material (liquid, solid, semi-solid or other substance) placed inside the body. In one aspect of these embodiments, the internal radiation therapy is brachytherapy, where a solid radioactive source is placed inside the body near the brain tumor. In another aspect of these embodiments, the internal radiation therapy comprises the systemic administration of a radiation source, typically a radionuclide (radioisotope or unsealed source). The radiation source may be orally administered or may be injected into a vein.

### Combination Therapies

In some embodiments, the methods described herein comprise the additional step of co-administering to a patient in need thereof an additional therapy.

In some embodiments, the medicament for use in treating a cancer characterized by the presence of an IDH1 and/or IDH2 mutation in a patient in need thereof is for use in combination with the co-administration of an additional therapy.

In another aspect, the solid form, i.e. pharmaceutically acceptable salt or cocrystal described herein, or pharmaceutical composition thereof for use in treating a cancer characterized by the presence of an IDH1 and/or IDH2 mutation is for use in combination with the co-administration of an additional therapy.

As used herein, the term “additional therapy” includes cancer therapies (including cancer therapeutic agents and cancer treatments), as described above, as well as non-cancer therapies (including non-cancer therapeutic agents and non-cancer treatments) administered to treat symptoms and/or secondary effects of the cancer. In other words, the term “additional therapy” includes additional therapeutic agents (i.e., cancer therapeutic agents and non-cancer therapeutic agents) and additional treatments (i.e., cancer treatments and non-cancer treatments).

In some embodiments, the additional therapy is a cancer therapy (i.e., a cancer therapeutic agent or cancer treatment), as described above.

In some embodiments, the additional therapy is a non-cancer therapy (i.e., a non-cancer therapeutic agent or non-cancer treatment).

In some embodiments, the additional therapy comprises one or more of a DNA-reactive agents, a PARP inhibitor, an anti-emesis agent, an anti-convulsant or anti-epileptic agent, a checkpoint inhibitor, PVC chemotherapy, bevacizumab, and gemcitabine.

In some embodiments, the additional therapy comprises a DNA-reactive agent. As used herein, “DNA-reactive agents” are those agents, such as alkylating agents, cross-linking agents, and DNA intercalating agents, which interact covalently or non-covalently with cellular DNA. For example, DNA-reactive agents include adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, mitozolomide, nedaplatin, oxaliplatin, piposulfan, procarbazine, semustine, streptozocin, temozolomide, thiotepa, treosulfan, diethylnitrosoamine, benzo (a) pyrene, doxorubicin, mitomycin-C, and the like. Many of these DNA-reactive agents are useful in cancer therapy as DNA-reactive chemotherapeutic agents.

In some embodiments, the additional therapy comprises a PARP inhibitor. As used herein, “PARP inhibitor” refers to an inhibitor of the enzyme poly ADP ribose polymerase (PARP). Examples of PARP inhibitors include pamiparib, olaparib, rucaparib, velaparib, iniparib, talazoparib, niraparib, and the like.

In some embodiments, the additional therapy is a checkpoint inhibitor. As used herein, “checkpoint inhibitor” refers to a therapeutic agent that inhibits an immune checkpoint (e.g., CTLA-4, PD-1/PD-L1, and the like) that otherwise would prevent immune system attacks on cancer cells, thereby allowing the immune system to attack the cancer cells.

Examples of check point inhibitors include ipilimumab, nivolumab, pembrolizumab, atezolizumab, avelumab, durvalumab, BGB-A317, spartalizumab, and the like.

In some embodiments, the additional therapy is PVC chemotherapy. As used herein, “PVC chemotherapy” refers to a chemotherapy regimen comprising the combined administration of procarbazine, lomustine (which is sold under the trade name CCNU®), and vincristine (which is sold under the trade name Onocovin®). Typically, the vincristine is administered intravenously, while the procarbazine, and lomustine are administered orally. PCV chemotherapy often is administered in cycles, wherein each cycle comprises a single administration of vincristine and lomustine and a 10-day course of treatment with procarbazine.

In some embodiments, the additional therapy is bevacizumab. Bevacizumab, which is sold under the trade name Avastin®, is a recombinant humanized monoclonal antibody.

In some embodiments, the additional therapy is gemcitabine. Gemcitabine, which is sold under the trade name Gemzar®, is a pyrimidine nucleoside analog.

In some embodiments, the additional therapy is a non-cancer therapeutic agent. As used herein, the term “non-cancer therapeutic agent” refers to a therapeutic agent that is used to treat symptoms suffered by patients afflicted with a cancer, and/or undergoing treatment for a cancer, but that is not indicated for treating the cancer itself. Examples of “non-cancer therapeutic agents” include anti-seizure and anti-epileptic agents, anti-emesis agents, anti-diarrheal agents, and the like.

In some embodiments, the additional therapy is an anti-seizure or anti-epileptic agent. As used herein, “anti-seizure or anti-epileptic agent” refers to a drug that is effective for treating or preventing seizures, including epileptic seizures. Examples of anti-seizure and anti-epileptic agents include acetazolamide, barbexaclone, beclamide, brivaracetam, cannabidiol, carbamazepine, clobazam, clonazepam, clorazepate, diazepam, divalproex sodium, eslicarbazepine acetate, ethadione, ethosuximide, ethotoin, etiracetam, felbamate, fosphenytoin, gabapentin, lacosamide, lamotrigine, levetiracetam, lorazepam, mephenytoin, mesuximide, methazolamide, methylphenobarbital, midazolam, nimetazepam, nitrazepam, oxcarbazepine, paraldehyde, paramethadoine, perampanel, piracetam, phenacemide, pheneturide, phenobarbital, phensuximide, phenytoin, potassium bromide, pregabalin, primidone, progabide, pyridoxine, rufinamide, seletracetam, sodium valproate, stiripentol, sultiame, temazepam, tiagabine, topiramate, trimethadione, valnoctamide, valproic acid, valpromide, vigabatrin, zonisamide, and the like.

In some embodiments, the additional therapy is an anti-emesis agent. As used herein, “anti-emesis agent” refers to a drug that is effective to reduce vomiting and nausea symptoms. Examples of anti-emesis agents include 5-HT₃ receptor antagonists (e.g., dolasetron, granisetron, ondansetron, tropisetron, palonosetron, mirtazapine, and the like), dopamine agonists (e.g., domperidone, olanzapine, droperidol, haloperidol, chlorpromazine, prochlorperazine, alizapride, prochlorperazine, metoclopramide, and the like), NK1 receptor antagonists (e.g., aprepitant, casopitant, rolapitant, and the like), antihistamines (e.g., cinnarizine, cyclizine, diphenhydramine, dimenhydrinate, doxylamine, meclizine, promethazine, hydroxyzine, and the like), cannabinoids (e.g, cannabis, dronabinol, synthetic cannabinoids, and the like), benzodiazepines (e.g., midazolam, lorazepam, and the like), anticholinergics (e.g., scopolamine and the like), steroids (e.g, dexamethasone and the like), trimethobenzamide, ginger, propofol, glucose/fructose/phosphoric acid (which is sold under the trade name Emetrol®), peppermint, muscimol, ajwain, bismuth-subsalicylate, and the like.

In some embodiments, the additional therapy is an anti-diarrheal agent. Examples of anti-diarrheal agents include bismuth subgallate, Saccharomyces boulardii lyo, atropine, diphenoxylate, difenoxin, Lactobacillus acidophilus, bismuth subsalicylate, loperamide, Lactobacillus bulgaricus, Lactobacillus rhamnosus gg, attapulgite, crofelemer, simethicone, and the like.

In some embodiments, the additional therapy is a non-cancer treatment. As used herein, the term “non-cancer treatment” refers to a treatment that is used to treat symptoms suffered by patients afflicted with a cancer, and/or undergoing treatment for a cancer, but that is not indicated for treating the cancer itself. Examples of non-cancer treatments include acupuncture, biofeedback, distraction, emotional support and counseling, hypnosis, imagery, relaxation, skin stimulation, and the like.

The term “co-administering” as used herein, means that the additional therapy is administered prior to, concurrently with, consecutively with, or following the administration of the solid form, i.e. pharmaceutically acceptable salt or cocrystal described herein, or pharmaceutical composition thereof as part of a treatment regimen to provide a beneficial effect from the combined action of the solid form, i.e. pharmaceutically acceptable salt or cocrystal described herein (or pharmaceutical composition thereof) and the additional therapy. Where the additional therapy is an additional therapeutic agent, the additional therapeutic agent may be administered together with the solid form, i.e. pharmaceutically acceptable salt or cocrystal described herein as part of a single dosage form (such as a composition of one aspect of this invention comprising a cocrystal, pharmaceutically acceptable salt, crystalline form of a pharmaceutically acceptable salt, or amorphous solid dispersion and the therapeutic agent) or as separate, multiple dosage forms. Alternatively, the therapeutic agent may be administered prior to, consecutively with, or following the administration of the solid form, i.e. pharmaceutically acceptable salt or cocrystal described herein. In such combination therapy treatment, both the solid form, i.e. pharmaceutically acceptable salt or cocrystal described herein and the additional therapeutic agent(s) are administered by conventional methods. The administration of a composition of one aspect of this invention, comprising both a solid form, i.e. pharmaceutically acceptable salt or cocrystal described herein and an additional therapeutic agent, to a patient does not preclude the separate administration of that same therapeutic agent, any other additional therapeutic agent or the solid form, i.e. pharmaceutically acceptable salt or cocrystal described herein to said patient at another time during a course of treatment. Where the additional therapy is an additional treatment, the additional treatment may be administered prior to, consecutively with, concurrently with or following the administration of the solid form, i.e. pharmaceutically acceptable salt or cocrystal described herein or pharmaceutical composition thereof.

In some embodiments, when the additional therapy is a cancer therapy, both the solid form, i.e. pharmaceutically acceptable salt or cocrystal described herein and the cancer therapy are administered at dosage levels of between about 1 to 100%, or between about 5 to 95%, of the dosage normally administered in a monotherapy regimen.

### ENUMERATED EMBODIMENTS

In some embodiments, the disclosure relates to:

1. A pharmaceutically acceptable salt of a compound of formula (I)

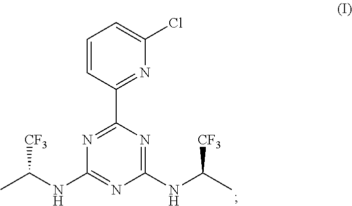 [SMILES: C[C@@H](Nc1nc(N[C@H](C)C(F)(F)F)nc(-c2cccc(Cl)n2)n1)C(F)(F)F]

characterized in that the pharmaceutically acceptable salt is formed using benzene sulfonic acid, (+)-camphor-10-sulfonic acid, ethane-1,2-disulfonic acid, ethanesulfonic acid, hydrobromic acid, hydrochloric acid, naphthalene-1,5-disulfonic acid, naphthalene-2-sulfonic acid, p-toluenesulfonic acid sulfuric acid or ortho phosphoric acid.

2. A pharmaceutically acceptable salt according to embodiment 1, characterized in that the pharmaceutically acceptable salt is crystalline.

3. A pharmaceutically acceptable salt according to embodiment 2, characterized in that the pharmaceutically acceptable salt is formed using benzene sulfonic acid and the X-ray powder diffraction pattern comprises at least two peak positions, in degrees 2-theta (±0.2 degrees 2-theta), selected from the group consisting of 5.83, 8.17, 14.40 and 17.90.

4. A pharmaceutically acceptable salt according to embodiment 2, characterized in that the pharmaceutically acceptable salt is formed using benzene sulfonic acid and the X-ray powder diffraction pattern comprises at least three peak positions, in degrees 2-theta (±0.2 degrees 2-theta), selected from the group consisting of 5.83, 8.17, 14.40 and 17.90.

5. A pharmaceutically acceptable salt according to embodiment 2, characterized in that the pharmaceutically acceptable salt is formed using benzene sulfonic acid and the X-ray powder diffraction pattern comprises the four following peak positions, in degrees 2-theta (±0.2 degrees 2-theta): 5.83, 8.17, 14.40 and 17.90.

6. A pharmaceutically acceptable salt according to embodiment 2, characterized in that the pharmaceutically acceptable salt is formed using (+)-camphor-10-sulfonic acid and the X-ray powder diffraction pattern comprises at least two peak positions, in degrees 2-theta (±0.2 degrees 2-theta), selected from the group consisting of 8.29, 10.19, 12.07, 18.12 and 19.97.

7. A pharmaceutically acceptable salt according to embodiment 2, characterized in that the pharmaceutically acceptable salt is formed using (+)-camphor-10-sulfonic acid and the X-ray powder diffraction pattern comprises at least three peak positions, in degrees 2-theta (±0.2 degrees 2-theta), selected from the group consisting of 8.29, 10.19, 12.07, 18.12 and 19.97.

8. A pharmaceutically acceptable salt according to embodiment 2, characterized in that the pharmaceutically acceptable salt is formed using (+)-camphor-10-sulfonic acid and the X-ray powder diffraction pattern comprises at least four peak positions, in degrees 2-theta (±0.2 degrees 2-theta), selected from the group consisting of 8.29, 10.19, 12.07, 18.12 and 19.97.

9. A pharmaceutically acceptable salt according to embodiment 2, characterized in that the pharmaceutically acceptable salt is formed using (+)-camphor-10-sulfonic acid and the X-ray powder diffraction pattern comprises peak positions, in degrees 2-theta (±0.2 degrees 2-theta), of 10.19 and 12.07, and at least one peak position selected from the group consisting of 8.29, 18.12 and 19.97.

10. A pharmaceutically acceptable salt according to embodiment 2, characterized in that the pharmaceutically acceptable salt is formed using ethane-1,2-disulfonic acid and the X-ray powder diffraction pattern comprises at least two peak positions, in degrees 2-theta (±0.2 degrees 2-theta), selected from the group consisting of 5.59, 14.75, 18.08 and 18.87.

11. A pharmaceutically acceptable salt according to embodiment 2, characterized in that the pharmaceutically acceptable salt is formed using ethane-1,2-disulfonic acid and the X-ray powder diffraction pattern comprises at least three peak positions, in degrees 2-theta (±0.2 degrees 2-theta), selected from the group consisting of 5.59, 14.75, 18.08 and 18.87.

12. A pharmaceutically acceptable salt according to embodiment 2, characterized in that the pharmaceutically acceptable salt is formed using ethane-1,2-disulfonic acid and the X-ray powder diffraction pattern comprises the following four peak positions, in degrees 2-theta (±0.2 degrees 2-theta): 5.59, 14.75, 18.08 and 18.87.

13. A pharmaceutically acceptable salt according to embodiment 2, characterized in that the pharmaceutically acceptable salt is formed using ethane-1,2-disulfonic acid and the X-ray powder diffraction pattern comprises peak positions, in degrees 2-theta (±0.2 degrees 2-theta), of 5.59 and 18.87, and at least one peak position selected from the group consisting of 14.75 and 18.08.

14. A pharmaceutically acceptable salt according to embodiment 10-13, characterized in that the ethane-1,2-disulfonic salt is characterized by a differential scanning calorimetry thermogram comprising an endothermic peak having an onset temperature of 304.30° C. (±2.0° C.).

15. A pharmaceutically acceptable salt according to embodiment 2, characterized in that the pharmaceutically acceptable salt is formed using ethanesulfonic acid and the X-ray powder diffraction pattern comprises at least two peak positions, in degrees 2-theta (±0.2 degrees 2-theta), selected from the group consisting of 6.86, 13.97, 14.27, 17.00 and 17.72.

16. A pharmaceutically acceptable salt according to embodiment 2, characterized in that the pharmaceutically acceptable salt is formed using ethanesulfonic acid and the X-ray powder diffraction pattern comprises at least three peak positions, in degrees 2-theta (±0.2 degrees 2-theta), selected from the group consisting of 6.86, 13.97, 14.27, 17.00 and 17.72.

17. A pharmaceutically acceptable salt according to embodiment 2, characterized in that the pharmaceutically acceptable salt is formed using ethanesulfonic acid and the X-ray powder diffraction pattern comprises at least four peak positions, in degrees 2-theta (±0.2 degrees 2-theta), selected from the group consisting of 6.86, 13.97, 14.27, 17.00 and 17.72.

18. A pharmaceutically acceptable salt according to embodiment 2, characterized in that the pharmaceutically acceptable salt is formed using ethanesulfonic acid and the X-ray powder diffraction pattern comprises peak positions, in degrees 2-theta (±0.2 degrees 2-theta), of 13.97 and 14.27, and at least one peak position selected from the group consisting of 6.86, 17.00 and 17.72.

19. A pharmaceutically acceptable salt according to embodiment 2, characterized in that the pharmaceutically acceptable salt is formed using hydrobromic acid and the X-ray powder diffraction pattern comprises at least two peak positions, in degrees 2-theta (±0.2 degrees 2-theta), selected from the group consisting of 5.74, 8.56, 13.63, 14.90 and 15.08.

20. A pharmaceutically acceptable salt according to embodiment 2, characterized in that the pharmaceutically acceptable salt is formed using hydrobromic acid and the X-ray powder diffraction pattern comprises at least three peak positions, in degrees 2-theta (±0.2 degrees 2-theta), selected from the group consisting of 5.74, 8.56, 13.63, 14.90 and 15.08.

21. A pharmaceutically acceptable salt according to embodiment 2, characterized in that the pharmaceutically acceptable salt is formed using hydrobromic acid and the X-ray powder diffraction pattern comprises at least four peak positions, in degrees 2-theta (±0.2 degrees 2-theta), selected from the group consisting of 5.74, 8.56, 13.63, 14.90 and 15.08.

22. A pharmaceutically acceptable salt according to embodiment 2, characterized in that the pharmaceutically acceptable salt is formed using hydrobromic acid and the X-ray powder diffraction pattern comprises peak positions, in degrees 2-theta (±0.2 degrees 2-theta), of 5.74 and 8.56, and at least one peak position selected from the group consisting of 13.63, 14.90 and 15.08.

23. A pharmaceutically acceptable salt according to embodiment 2, characterized in that the pharmaceutically acceptable salt is formed using hydrochloric acid and the X-ray powder diffraction pattern comprises at least two peak positions, in degrees 2-theta (±0.2 degrees 2-theta), selected from the group consisting of 5.73, 9.71, 13.78, 14.64 and 16.25.

24. A pharmaceutically acceptable salt according to embodiment 2, characterized in that the pharmaceutically acceptable salt is formed using hydrochloric acid and the X-ray powder diffraction pattern comprises at least three peak positions, in degrees 2-theta (±0.2 degrees 2-theta), selected from the group consisting of 5.73, 9.71, 13.78, 14.64 and 16.25.

25. A pharmaceutically acceptable salt according to embodiment 2, characterized in that the pharmaceutically acceptable salt is formed using hydrochloric acid and the X-ray powder diffraction pattern comprises at least four peak positions, in degrees 2-theta (±0.2 degrees 2-theta), selected from the group consisting of 5.73, 9.71, 13.78, 14.64 and 16.25.

26. A pharmaceutically acceptable salt according to embodiment 2, characterized in that the pharmaceutically acceptable salt is formed using hydrochloric acid and the X-ray powder diffraction pattern comprises peak positions, in degrees 2-theta (±0.2 degrees 2-theta), of 5.73 and 14.64, and at least one peak position selected from the group consisting of 9.71, 13.78 and 16.25.

27. A pharmaceutically acceptable salt according to embodiment 2, characterized in that the pharmaceutically acceptable salt is formed using naphthalene-1,5-disulfonic acid and the X-ray powder diffraction pattern comprises at least two peak positions, in degrees 2-theta (±0.2 degrees 2-theta), selected from the group consisting of 5.52, 13.79, 14.17, 19.28, 19.63 and 19.97.

28. A pharmaceutically acceptable salt according to embodiment 2, characterized in that the pharmaceutically acceptable salt is formed using naphthalene-1,5-disulfonic acid and the X-ray powder diffraction pattern comprises at least three peak positions, in degrees 2-theta (±0.2 degrees 2-theta), selected from the group consisting of 5.52, 13.79, 14.17, 19.28, 19.63 and 19.97.

29. A pharmaceutically acceptable salt according to embodiment 2, characterized in that the pharmaceutically acceptable salt is formed using naphthalene-1,5-disulfonic acid and the X-ray powder diffraction pattern comprises at least four peak positions, in degrees 2-theta (±0.2 degrees 2-theta), selected from the group consisting of 5.52, 13.79, 14.17, 19.28, 19.63 and 19.97.

30. A pharmaceutically acceptable salt according to embodiment 2, characterized in that the pharmaceutically acceptable salt is formed using naphthalene-1,5-disulfonic acid and the X-ray powder diffraction pattern comprises peak positions, in degrees 2-theta (±0.2 degrees 2-theta), of 5.52 and 19.97, and at least one peak position selected from the group consisting of 13.79, 14.17, 19.28 and 19.63.

31. A pharmaceutically acceptable salt according to embodiment 27-30, characterized in that the naphthalene-1,5-disulfonic salt is characterized by a differential scanning calorimetry thermogram comprising an endothermic peak having an onset temperature of 324.30° C. (±2.0° C.).

32. A pharmaceutically acceptable salt according to embodiment 2, characterized in that the pharmaceutically acceptable salt is formed using naphthalene-2-sulfonic acid and the X-ray powder diffraction pattern comprises at least two peak positions, in degrees 2-theta (±0.2 degrees 2-theta), selected from the group consisting of 8.41, 13.65, 26.09 and 26.47.

33. A pharmaceutically acceptable salt according to embodiment 2, characterized in that the pharmaceutically acceptable salt is formed using naphthalene-2-sulfonic acid and the X-ray powder diffraction pattern comprises at least three peak positions, in degrees 2-theta (±0.2 degrees 2-theta), selected from the group consisting of 8.41, 13.65, 26.09 and 26.47.

34. A pharmaceutically acceptable salt according to embodiment 2, characterized in that the pharmaceutically acceptable salt is formed using naphthalene-2-sulfonic acid and the X-ray powder diffraction pattern comprises the following four peak positions, in degrees 2-theta (±0.2 degrees 2-theta) selected from the group consisting of 8.41, 13.65, 26.09 and 26.47.

35. A pharmaceutically acceptable salt according to embodiment 2, characterized in that the pharmaceutically acceptable salt is formed using naphthalene-2-sulfonic acid and the X-ray powder diffraction pattern comprises peak positions, in degrees 2-theta (±0.2 degrees 2-theta), of 8.41 and 26.47, and at least one peak position selected from the group consisting of 13.65 and 26.09.

36. A pharmaceutically acceptable salt according to embodiment 2, characterized in that the pharmaceutically acceptable salt is formed using p-toluenesulfonic acid and the X-ray powder diffraction pattern comprises at least two peak positions, in degrees 2-theta (±0.2 degrees 2-theta), selected from the group consisting of 5.29, 8.37, 8.66, 12.96, 13.47, 20.42 and 20.64.

37. A pharmaceutically acceptable salt according to embodiment 2, characterized in that the pharmaceutically acceptable salt is formed using p-toluenesulfonic acid and the X-ray powder diffraction pattern comprises at least three peak positions, in degrees 2-theta (±0.2 degrees 2-theta), selected from the group consisting of 5.29, 8.37, 8.66, 12.96, 13.47, 20.42 and 20.64.

38. A pharmaceutically acceptable salt according to embodiment 2, characterized in that the pharmaceutically acceptable salt is formed using p-toluenesulfonic acid and the X-ray powder diffraction pattern comprises at least four peak positions, in degrees 2-theta (±0.2 degrees 2-theta), selected from the group consisting of 5.29, 8.37, 8.66, 12.96, 13.47, 20.42 and 20.64.

39. A pharmaceutically acceptable salt according to embodiment 2, characterized in that the pharmaceutically acceptable salt is formed using p-toluenesulfonic acid and the X-ray powder diffraction pattern comprises peak positions, in degrees 2-theta (±0.2 degrees 2-theta), of 5.29 and 12.96, and at least one peak position selected from the group consisting of 8.37, 8.66, 13.47, 20.42 and 20.64.

40. A pharmaceutically acceptable salt according to embodiment 2, characterized in that the pharmaceutically acceptable salt is formed using sulfuric acid and the X-ray powder diffraction pattern comprises at least two peak positions, in degrees 2-theta (±0.2 degrees 2-theta), selected from the group consisting of 5.80, 8.50, 13.62, 14.21, 14.87, and 18.04.

41. A pharmaceutically acceptable salt according to embodiment 2, characterized in that the pharmaceutically acceptable salt is formed using sulfuric acid and the X-ray powder diffraction pattern comprises at least three peak positions, in degrees 2-theta (±0.2 degrees 2-theta), selected from the group consisting of 5.80, 8.50, 13.62, 14.21, 14.87, and 18.04.

42. A pharmaceutically acceptable salt according to embodiment 2, characterized in that the pharmaceutically acceptable salt is formed using sulfuric acid and the X-ray powder diffraction pattern comprises at least four peak positions, in degrees 2-theta (±0.2 degrees 2-theta), selected from the group consisting of 5.80, 8.50, 13.62, 14.21, 14.87, and 18.04.

43. A pharmaceutically acceptable salt according to embodiment 2, characterized in that the pharmaceutically acceptable salt is formed using sulfuric acid and the X-ray powder diffraction pattern comprises peak positions, in degrees 2-theta (±0.2 degrees 2-theta), of 5.80 and 14.87, and at least one peak position selected from the group consisting of 8.50, 13.62, 14.21 and 18.04.

44. A pharmaceutically acceptable salt according to embodiment 2, characterized in that the pharmaceutically acceptable salt is formed using ortho phosphoric acid and the X-ray powder diffraction pattern comprises at least two peak positions, in degrees 2-theta (±0.2 degrees 2-theta), selected from the group consisting of 5.47, 14.27, 14.91, 18.35, 19.72 and 21.29.

45. A pharmaceutically acceptable salt according to embodiment 2, characterized in that the pharmaceutically acceptable salt is formed using ortho phosphoric acid and the X-ray powder diffraction pattern comprises at least three peak positions, in degrees 2-theta (±0.2 degrees 2-theta), selected from the group consisting of 5.47, 14.27, 14.91, 18.35, 19.72 and 21.29.

46. A pharmaceutically acceptable salt according to embodiment 2, characterized in that the pharmaceutically acceptable salt is formed using ortho phosphoric acid and the X-ray powder diffraction pattern comprises at least four peak positions, in degrees 2-theta (±0.2 degrees 2-theta), selected from the group consisting of 5.47, 14.27, 14.91, 18.35, 19.72 and 21.29.

47. A pharmaceutically acceptable salt according to embodiment 2, characterized in that the pharmaceutically acceptable salt is formed using ortho phosphoric acid and the X-ray powder diffraction pattern comprises peak positions, in degrees 2-theta (±0.2 degrees 2-theta), of 18.35 and 21.29, and at least one peak position selected from the group consisting of 5.47, 14.27, 14.91 and 19.72.

48. A pharmaceutical composition comprising a pharmaceutically acceptable salt according to any one of embodiments 1-47 and one or more pharmaceutical excipients.

49. The pharmaceutical composition of embodiment 48, characterized in that the pharmaceutical composition comprises 1-10% w/w of the compound of formula (I).

50. The pharmaceutical composition of embodiment 48 or 49, characterized in that the pharmaceutical composition is in the form of an orally acceptable dosage form and comprises about 10 mg, about 20 mg, about 25 mg, about 40 mg, about 50 mg, about 100 mg, about 200 mg, or about 300 mg of the compound of formula (I).

51. The pharmaceutical composition of embodiment 50, characterized in that the pharmaceutical composition comprises about 10 mg or about 40 mg of the compound of formula (I).

52. The pharmaceutical composition of embodiment 48, characterized in that the pharmaceutical composition comprises 20-30% w/w of the compound of formula (I).

53. The pharmaceutical composition of embodiment 52, characterized in that the pharmaceutical composition is in the form of an orally acceptable dosage form and comprises about 10 mg, about 20 mg, about 25 mg, about 40 mg, about 50 mg, about 100 mg, about 200 mg, or about 300 mg of the compound of formula (I).

54. An amorphous solid dispersion prepared from a pharmaceutically acceptable salt according to anyone of embodiments 1-47 and a polymer.

55. An amorphous solid dispersion prepared from a pharmaceutically acceptable salt according to embodiment 54, characterized in that the polymer is selected from the group consisting of hydroxypropyl methyl cellulose (HPMC), hydroxypropyl methylcellulose acetate succinate (HPMCAS), hydroxypropyl methyl cellulose phthalate (HPMCP), hydroxypropyl cellulose (HPC), ethylcellulose, cellulose acetate phthalate, and polyvinylpyrrolidone (PVP), or a mixture thereof.

56. An amorphous solid dispersion prepared from a pharmaceutically acceptable salt according to embodiment 55, characterized in that the polymer is HPMCAS.

57. A pharmaceutical composition comprising a solid dispersion according to any one of embodiments 54 to 56 and one or more acceptable excipients.

58. A cocrystal comprising a compound of formula (I)

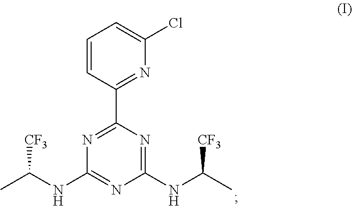 [SMILES: C[C@@H](Nc1nc(N[C@H](C)C(F)(F)F)nc(-c2cccc(Cl)n2)n1)C(F)(F)F]

and one coformer selected from 3-hydroxy-2-naphtoic acid, L-serine, glycine, D-gluconic acid, glycolic acid, L-malic acid, oxalic acid, benzoic acid, fumaric acid, gentisic acid, glutaric acid, 4-hydroxybenzoic acid, alpha-ketoglutaric acid, malonic acid, salicylic acid, L-tartaric acid, urea, pyroglutamic avid, caproic acid, glycerol, L-lysine, S-proline and pyruvic acid.

59. A cocrystal according to embodiment 58, characterized in that the coformer is 3-hydroxy-2-naphtoic acid and the X-ray powder diffraction pattern comprises at least two peak positions, in degrees 2-theta (±0.2 degrees 2-theta), selected from the group consisting of 6.41, 13.34, 13.69, 15.84 and 20.21.

60. A cocrystal according to embodiment 58, characterized in that the coformer is 3-hydroxy-2-naphtoic acid and the X-ray powder diffraction pattern comprises at least three peak positions, in degrees 2-theta (±0.2 degrees 2-theta), selected from the group consisting of 6.41, 13.34, 13.69, 15.84 and 20.21.

61. A cocrystal according to embodiment 58, characterized in that the coformer is 3-hydroxy-2-naphtoic acid and the X-ray powder diffraction pattern comprises at least four peak positions, in degrees 2-theta (±0.2 degrees 2-theta), selected from the group consisting of 6.41, 13.34, 13.69, 15.84 and 20.21.

62. A cocrystal according to embodiment 58, characterized in that the coformer is 3-hydroxy-2-naphtoic acid and the X-ray powder diffraction pattern comprises peak positions, in degrees 2-theta (±0.2 degrees 2-theta), of 6.41 and 13.34, and at least one peak position selected from the group consisting of 13.69, 15.84 and 20.21.

63. A cocrystal according to any one of embodiments 59 to 62, characterized in that the cocrystal is characterized by a differential scanning calorimetry thermogram comprising an endothermic peak having an onset temperature of 202.79° C. (±2.0° C.).

64. A cocrystal according to embodiment 58, characterized in that the coformer is L-serine and the X-ray powder diffraction pattern comprises at least two peak positions, in degrees 2-theta (±0.2 degrees 2-theta), selected from the group consisting of 5.90, 8.46, 13.28, 13.88 and 18.72.

65. A cocrystal according to embodiment 58, characterized in that the coformer is L-serine and the X-ray powder diffraction pattern comprises at least three peak positions, in degrees 2-theta (±0.2 degrees 2-theta), selected from the group consisting of 5.90, 8.46, 13.28, 13.88 and 18.72.

66. A cocrystal according to embodiment 58, characterized in that the coformer is L-serine and the X-ray powder diffraction pattern comprises at least four peak positions, in degrees 2-theta (±0.2 degrees 2-theta), selected from the group consisting of 5.90, 8.46, 13.28, 13.88 and 18.72.

67. A cocrystal according to embodiment 58, characterized in that the coformer is L-serine and the X-ray powder diffraction pattern comprises peak positions, in degrees 2-theta (±0.2 degrees 2-theta), of 8.46 and 13.88, and at least one peak position selected from the group consisting of 5.90, 13.28 and 18.72.

68. A cocrystal according to embodiment 58, characterized in that the coformer is glycine and the X-ray powder diffraction pattern comprises at least two peak positions, in degrees 2-theta (±0.2 degrees 2-theta), selected from the group consisting of 8.62, 14.04, 14.79 and 26.04.

69. A cocrystal according to embodiment 58, characterized in that the coformer is glycine and the X-ray powder diffraction pattern comprises at least three peak positions, in degrees 2-theta (±0.2 degrees 2-theta), selected from the group consisting of 8.62, 14.04, 14.79 and 26.04.

70. A cocrystal according to embodiment 58, characterized in that the coformer is glycine and the X-ray powder diffraction pattern comprises the following four peak positions, in degrees 2-theta (±0.2 degrees 2-theta): 8.62, 14.04, 14.79 and 26.04.

71. A cocrystal according to embodiment 58, characterized in that the coformer is D-gluconic acid and the X-ray powder diffraction pattern comprises at least two peak positions, in degrees 2-theta (±0.2 degrees 2-theta), selected from the group consisting of 6.17, 14.14, 15.32, 19.17 and 19.32.

72. A cocrystal according to embodiment 58, characterized in that the coformer is D-gluconic acid and the X-ray powder diffraction pattern comprises at least three peak positions, in degrees 2-theta (±0.2 degrees 2-theta), selected from the group consisting of 6.17, 14.14, 15.32, 19.17 and 19.32.

73. A cocrystal according to embodiment 58, characterized in that the coformer is D-gluconic acid and the X-ray powder diffraction pattern comprises at least four peak positions, in degrees 2-theta (±0.2 degrees 2-theta), selected from the group consisting of 6.17, 14.14, 15.32, 19.17 and 19.32.

74. A cocrystal according to embodiment 58, characterized in that the coformer is D-gluconic acid and the X-ray powder diffraction pattern comprises peak positions, in degrees 2-theta (±0.2 degrees 2-theta), of 15.32 and 19.32, and at least one peak position selected from the group consisting of 6.17, 14.14 and 19.17.

75. A cocrystal according to embodiment 58, characterized in that the coformer is glycolic acid and the X-ray powder diffraction pattern comprises at least two peak positions, in degrees 2-theta (±0.2 degrees 2-theta), selected from the group consisting of 7.90, 14.04 and 16.25.

76. A cocrystal according to embodiment 58, characterized in that the coformer is glycolic acid and the X-ray powder diffraction pattern comprises the following three peak positions, in degrees 2-theta (±0.2 degrees 2-theta): 7.90, 14.04 and 16.25.

77. A cocrystal according to embodiment 58, characterized in that the coformer is L-malic acid and the X-ray powder diffraction pattern comprises at least two peak positions, in degrees 2-theta (±0.2 degrees 2-theta), selected from the group consisting of 5.87, 8.49, 14.13, 15.40 and 19.55.

78. A cocrystal according to embodiment 58, characterized in that the coformer is L-malic acid and the X-ray powder diffraction pattern comprises at least three peak positions, in degrees 2-theta (±0.2 degrees 2-theta), selected from the group consisting of 5.87, 8.49, 14.13, 15.40 and 19.55.

79. A cocrystal according to embodiment 58, characterized in that the coformer is L-malic acid and the X-ray powder diffraction pattern comprises at least four peak positions, in degrees 2-theta (±0.2 degrees 2-theta), selected from the group consisting of 5.87, 8.49, 14.13, 15.40 and 19.55.

80. A cocrystal according to embodiment 58, characterized in that the coformer is L-malic acid and the X-ray powder diffraction pattern comprises peak positions, in degrees 2-theta (±0.2 degrees 2-theta), of 5.87 and 14.13, and at least one peak position selected from the group consisting of 8.49, 15.40 and 19.55.

81. A cocrystal according to embodiment 58, characterized in that the coformer is oxalic acid and the X-ray powder diffraction pattern comprises at least two peak positions, in degrees 2-theta (±0.2 degrees 2-theta), selected from the group consisting of 5.81, 13.67 and 14.90.

82. A cocrystal according to embodiment 58, characterized in that the coformer is oxalic acid and the X-ray powder diffraction pattern comprises the following three peak positions, in degrees 2-theta (±0.2 degrees 2-theta): 5.81, 13.67 and 14.90.

83. A cocrystal according to embodiment 57, characterized in that the coformer is benzoic acid and the X-ray powder diffraction pattern comprises at least two peak positions, in degrees 2-theta (±0.2 degrees 2-theta), selected from the group consisting of 6.00, 8.50, 9.80, 18.05, and 19.99.

84. A cocrystal according to embodiment 58, characterized in that the coformer is benzoic acid and the X-ray powder diffraction pattern comprises at least three peak positions, in degrees 2-theta (±0.2 degrees 2-theta), selected from the group consisting of 6.00, 8.50, 9.80, 18.05, and 19.99.

85. A cocrystal according to embodiment 58, characterized in that the coformer is benzoic acid and the X-ray powder diffraction pattern comprises at least four peak positions, in degrees 2-theta (±0.2 degrees 2-theta), selected from the group consisting of 6.00, 8.50, 9.80, 18.05, and 19.99.

86. A cocrystal according to embodiment 58, characterized in that the coformer is benzoic acid and the X-ray powder diffraction pattern comprises peak positions, in degrees 2-theta (±0.2 degrees 2-theta), of 6.00 and 18.05, and at least one peak position selected from the group consisting of 8.50, 9.80, and 19.99.

87. A cocrystal according to any one of embodiments 83-86, characterized in that the cocrystal is characterized by a differential scanning calorimetry thermogram comprising an endothermic peak having an onset temperature of 128.06° C. (±2.0° C.).

88. A cocrystal according to embodiment 58, characterized in that the coformer is fumaric acid and the X-ray powder diffraction pattern comprises at least two peak positions, in degrees 2-theta (±0.2 degrees 2-theta), selected from the group consisting of 5.59, 9.45, 13.63, 14.25 and 19.46.

89. A cocrystal according to embodiment 58, characterized in that the conformer is fumaric acid and the X-ray powder diffraction pattern comprises at least three peak positions, in degrees 2-theta (±0.2 degrees 2-theta), selected from the group consisting of 5.59, 9.45, 13.63, 14.25 and 19.46.

90. A cocrystal according to embodiment 58, characterized in that the coformer is fumaric acid and the X-ray powder diffraction pattern comprises at least four peak positions, in degrees 2-theta (±0.2 degrees 2-theta), selected from the group consisting of 5.59, 9.45, 13.63, 14.25 and 19.46.

91. A cocrystal according to embodiment 58, characterized in that the coformer is fumaric acid and the X-ray powder diffraction pattern comprises peak positions, in degrees 2-theta (±0.2 degrees 2-theta), of 5.59 and 14.25, and at least one peak position selected from the group consisting of 9.45, 13.36 and 19.46.

92. A cocrystal according to embodiment 58, characterized in that the coformer is gentisic acid and the X-ray powder diffraction pattern comprises at least two peak positions, in degrees 2-theta (±0.2 degrees 2-theta), selected from the group consisting of 6.46, 12.63, 13.84, 17.34 and 26.08.

93. A cocrystal according to embodiment 58, characterized in that the coformer is gentisic acid and the X-ray powder diffraction pattern comprises at least three peak positions, in degrees 2-theta (±0.2 degrees 2-theta), selected from the group consisting of 6.46, 12.63, 13.84, 17.34 and 26.08.

94. A cocrystal according to embodiment 58, characterized in that the coformer is gentisic acid and the X-ray powder diffraction pattern comprises at least four peak positions, in degrees 2-theta (±0.2 degrees 2-theta), selected from the group consisting of 6.46, 12.63, 13.84, 17.34 and 26.08.

95. A cocrystal according to embodiment 58, characterized in that the coformer is gentisic acid and the X-ray powder diffraction pattern comprises peak positions, in degrees 2-theta (±0.2 degrees 2-theta), of 13.84 and 26.08, and at least one peak position selected from the group consisting of 6.46, 12.63 and 17.34.

96. A cocrystal according to embodiment 58, characterized in that the coformer is glutaric acid and the X-ray powder diffraction pattern comprises at least two peak positions, in degrees 2-theta (±0.2 degrees 2-theta), selected from the group consisting of 6.72, 7.19, 12.00, 12.65 and 15.23.

97. A cocrystal according to embodiment 58, characterized in that the coformer is glutaric acid and the X-ray powder diffraction pattern comprises at least three peak positions, in degrees 2-theta (±0.2 degrees 2-theta), selected from the group consisting of 6.72, 7.19, 12.00, 12.65 and 15.23.

98. A cocrystal according to embodiment 58, characterized in that the coformer is glutaric acid and the X-ray powder diffraction pattern comprises at least four peak positions, in degrees 2-theta (±0.2 degrees 2-theta), selected from the group consisting of 6.72, 7.19, 12.00, 12.65 and 15.23.

99. A cocrystal according to embodiment 58, characterized in that the coformer is glutaric acid and the X-ray powder diffraction pattern comprises peak positions, in degrees 2-theta (±0.2 degrees 2-theta), of 6.72 and 7.19, and at least one peak position selected from the group consisting of 12.00, 12.65 and 15.23.

100. A cocrystal according to embodiment 58, characterized in that the coformer is 4-hydroxybenzoic acid and the X-ray powder diffraction pattern comprises at least two peak positions, in degrees 2-theta (±0.2 degrees 2-theta), selected from the group consisting of 6.67, 14.64, 17.52, 20.59, 26.40 and 26.88.

101. A cocrystal according to embodiment 58, characterized in that the coformer is 4-hydroxybenzoic acid and the X-ray powder diffraction pattern comprises at least three peak positions, in degrees 2-theta (±0.2 degrees 2-theta), selected from the group consisting of 6.67, 14.64, 17.52, 20.59, 26.40 and 26.88.

102. A cocrystal according to embodiment 58, characterized in that the coformer is 4-hydroxybenzoic acid and the X-ray powder diffraction pattern comprises at least four peak positions, in degrees 2-theta (±0.2 degrees 2-theta), selected from the group consisting of 6.67, 14.64, 17.52, 20.59, 26.40 and 26.88.

103. A cocrystal according to embodiment 58, characterized in that the coformer is 4-hydroxybenzoic acid and the X-ray powder diffraction pattern comprises peak positions, in degrees 2-theta (±0.2 degrees 2-theta), of 6.67 and 14.64, and at least one peak position selected from the group consisting of 17.52, 20.59, 26.40 and 26.88.

104. A cocrystal according to embodiment 58, characterized in that the coformer is alpha-ketoglutaric acid and the X-ray powder diffraction pattern comprises at least two peak positions, in degrees 2-theta (±0.2 degrees 2-theta), selected from the group consisting of 5.89, 7.39, 17.30 and 26.89.

105. A cocrystal according to embodiment 58, characterized in that the coformer is alpha-ketoglutaric acid and the X-ray powder diffraction pattern comprises at least three peak positions, in degrees 2-theta (±0.2 degrees 2-theta), selected from the group consisting of 5.89, 7.39, 17.30 and 26.89.

106. A cocrystal according to embodiment 58, characterized in that the coformer is alpha-ketoglutaric acid and the X-ray powder diffraction pattern comprises the following four peak positions, in degrees 2-theta (±0.2 degrees 2-theta), selected from the group consisting of 5.89, 7.39, 17.30 and 26.89.

107. A cocrystal according to embodiment 58, characterized in that the coformer is alpha-ketoglutaric acid and the X-ray powder diffraction pattern comprises peak positions, in degrees 2-theta (±0.2 degrees 2-theta), of 5.89 and 17.30, and at least one peak position selected from the group consisting of 7.39 and 26.89.

108. A cocrystal according to embodiment 58, characterized in that the coformer is malonic acid and the X-ray powder diffraction pattern comprises at least two peak positions, in degrees 2-theta (±0.2 degrees 2-theta), selected from the group consisting of 7.97, 13.74, 16.07 and 19.34.

109. A cocrystal according to embodiment 58, characterized in that the coformer is malonic acid and the X-ray powder diffraction pattern comprises at least three peak positions, in degrees 2-theta (±0.2 degrees 2-theta), selected from the group consisting of 7.97, 13.74, 16.07 and 19.34.

110. A cocrystal according to embodiment 58, characterized in that the coformer is malonic acid and the X-ray powder diffraction pattern comprises the following four peak positions, in degrees 2-theta (±0.2 degrees 2-theta), selected from the group consisting of 7.97, 13.74, 16.07 and 19.34.

111. A cocrystal according to embodiment 58, characterized in that the coformer is malonic acid and the X-ray powder diffraction pattern comprises peak positions, in degrees 2-theta (±0.2 degrees 2-theta), of 7.97 and 19.34, and at least one peak position selected from the group consisting of 13.74 and 16.07.

112. A cocrystal according to embodiment 58, characterized in that the coformer is salicylic acid and the X-ray powder diffraction pattern comprises at least two peak positions, in degrees 2-theta (±0.2 degrees 2-theta), selected from the group consisting of 6.77, 7.29, 16.36, 18.24 and 21.21.

113. A cocrystal according to embodiment 58, characterized in that the coformer is salicylic acid and the X-ray powder diffraction pattern comprises at least three peak positions, in degrees 2-theta (±0.2 degrees 2-theta), selected from the group consisting of 6.77, 7.29, 16.36, 18.24 and 21.21.

114. A cocrystal according to embodiment 58, characterized in that the coformer is salicylic acid and the X-ray powder diffraction pattern comprises at least four peak positions, in degrees 2-theta (±0.2 degrees 2-theta), selected from the group consisting of 6.77, 7.29, 16.36, 18.24 and 21.21.

115. A cocrystal according to embodiment 58, characterized in that the coformer is salicylic acid and the X-ray powder diffraction pattern comprises peak positions, in degrees 2-theta (±0.2 degrees 2-theta), of 6.77 and 16.36, and at least one peak position selected from the group consisting of 7.29, 18.24 and 21.21.

116. A cocrystal according to embodiment 58, characterized in that the coformer is L-tartaric acid and the X-ray powder diffraction pattern comprises at least two peak positions, in degrees 2-theta (±0.2 degrees 2-theta), selected from the group consisting of 5.74, 11.84, 13.62 and 17.98.

117. A cocrystal according to embodiment 58, characterized in that the coformer is L-tartaric acid and the X-ray powder diffraction pattern comprises at least three peak positions, in degrees 2-theta (±0.2 degrees 2-theta), selected from the group consisting of 5.74, 11.84, 13.62 and 17.98.

118. A cocrystal according to embodiment 58, characterized in that the coformer is L-tartaric acid and the X-ray powder diffraction pattern comprises the following four peak positions, in degrees 2-theta (±0.2 degrees 2-theta), selected from the group consisting of 5.74, 11.84, 13.62 and 17.98.

119. A cocrystal according to embodiment 58, characterized in that the coformer is L-tartaric acid and the X-ray powder diffraction pattern comprises peak positions, in degrees 2-theta (±0.2 degrees 2-theta), of 13.62 and 17.98, and at least one peak position selected from the group consisting of 5.74 and 11.84.

120. A cocrystal according to embodiment 58, characterized in that the coformer is urea and the X-ray powder diffraction pattern comprises at least two peak positions, in degrees 2-theta (±0.2 degrees 2-theta), selected from the group consisting of 8.79, 13.76, 16.34 and 26.43.

121. A cocrystal according to embodiment 58, characterized in that the coformer is urea and the X-ray powder diffraction pattern comprises at least three peak positions, in degrees 2-theta (±0.2 degrees 2-theta), selected from the group consisting of 8.79, 13.76, 16.34 and 26.43.

122. A cocrystal according to embodiment 58, characterized in that the coformer is urea and the X-ray powder diffraction pattern comprises the following four peak positions, in degrees 2-theta (±0.2 degrees 2-theta), selected from the group consisting of 8.79, 13.76, 16.34 and 26.43.

123. A cocrystal according to embodiment 58, characterized in that the coformer is urea and the X-ray powder diffraction pattern comprises peak positions, in degrees 2-theta (±0.2 degrees 2-theta), of 8.79 and 13.76, and at least one peak position selected from the group consisting of 16.34 and 26.43.

124. A cocrystal according to embodiment 58, characterized in that the coformer is pyroglutamic acid and the X-ray powder diffraction pattern comprises at least two peak positions, in degrees 2-theta (±0.2 degrees 2-theta), selected from the group consisting of 6.20, 13.87, 25.30, 26.24 and 27.26.

125. A cocrystal according to embodiment 58, characterized in that the coformer is pyroglutamic acid and the X-ray powder diffraction pattern comprises at least three peak positions, in degrees 2-theta (±0.2 degrees 2-theta), selected from the group consisting of 6.20, 13.87, 25.30, 26.24 and 27.26.

126. A cocrystal according to embodiment 58, characterized in that the coformer is pyroglutamic acid and the X-ray powder diffraction pattern comprises at least four peak positions, in degrees 2-theta (±0.2 degrees 2-theta), selected from the group consisting of 6.20, 13.87, 25.30, 26.24 and 27.26.

127. A cocrystal according to embodiment 58, characterized in that the coformer is pyroglutamic acid and the X-ray powder diffraction pattern comprises peak positions, in degrees 2-theta (±0.2 degrees 2-theta), of 6.20 and 13.87, and at least one peak position selected from the group consisting of 25.30, 26.24 and 27.26.

128. A cocrystal according to embodiment 58, characterized in that the coformer is caproic acid and the X-ray powder diffraction pattern comprises at least two peak positions, in degrees 2-theta (±0.2 degrees 2-theta), selected from the group consisting of 8.55, 9.80, 14.25 and 23.10.

129. A cocrystal according to embodiment 58, characterized in that the coformer is caproic acid and the X-ray powder diffraction pattern comprises at least three peak positions, in degrees 2-theta (±0.2 degrees 2-theta), selected from the group consisting of 8.55, 9.80, 14.25 and 23.10.

130. A cocrystal according to embodiment 58, characterized in that the coformer is caproic acid and the X-ray powder diffraction pattern comprises the following four peak positions, in degrees 2-theta (±0.2 degrees 2-theta), selected from the group consisting of 8.55, 9.80, 14.25 and 23.10.

131. A cocrystal according to embodiment 58, characterized in that the coformer is caproic acid and the X-ray powder diffraction pattern comprises peak positions, in degrees 2-theta (±0.2 degrees 2-theta), of 8.55 and 14.25, and at least one peak position selected from the group consisting of 9.80 and 23.10.

132. A cocrystal according to embodiment 58, characterized in that the coformer is glycerol and the X-ray powder diffraction pattern comprises at least two peak positions, in degrees 2-theta (±0.2 degrees 2-theta), selected from the group consisting of 5.91, 8.41, 11.85, 14.32 and 17.99.

133. A cocrystal according to embodiment 58, characterized in that the coformer is glycerol and the X-ray powder diffraction pattern comprises at least three peak positions, in degrees 2-theta (±0.2 degrees 2-theta), selected from the group consisting of 5.91, 8.41, 11.85, 14.32 and 17.99.

134. A cocrystal according to embodiment 58, characterized in that the coformer is glycerol and the X-ray powder diffraction pattern comprises at least four peak positions, in degrees 2-theta (±0.2 degrees 2-theta), selected from the group consisting of 5.91, 8.41, 11.85, 14.32 and 17.99.

135. A cocrystal according to embodiment 58, characterized in that the coformer is glycerol and the X-ray powder diffraction pattern comprises peak positions, in degrees 2-theta (±0.2 degrees 2-theta), of 11.85 and 17.99, and at least one peak position selected from the group consisting of 5.91, 8.41 and 14.32.

136. A cocrystal according to embodiment 58, characterized in that the coformer is L-lysine and the X-ray powder diffraction pattern comprises at least two peak positions, in degrees 2-theta (±0.2 degrees 2-theta), selected from the group consisting of 5.00, 6.68, 12.61, 18.50 and 23.45.

137. A cocrystal according to embodiment 58, characterized in that the coformer is L-lysine and the X-ray powder diffraction pattern comprises at least three peak positions, in degrees 2-theta (±0.2 degrees 2-theta), selected from the group consisting of 5.00, 6.68, 12.61, 18.50 and 23.45.

138. A cocrystal according to embodiment 58, characterized in that the coformer is L-lysine and the X-ray powder diffraction pattern comprises at least four peak positions, in degrees 2-theta (±0.2 degrees 2-theta), selected from the group consisting of 5.00, 6.68, 12.61, 18.50 and 23.45.

139. A cocrystal according to embodiment 58, characterized in that the coformer is L-lysine and the X-ray powder diffraction pattern comprises peak positions, in degrees 2-theta (±0.2 degrees 2-theta), of 5.00 and 18.50, and at least one peak position selected from the group consisting of 6.68, 12.61 and 23.45.

140. A cocrystal according to embodiment 58, characterized in that the coformer is S-proline and the X-ray powder diffraction pattern comprises at least two peak positions, in degrees 2-theta (±0.2 degrees 2-theta), selected from the group consisting of 5.91, 7.19, 14.02 and 26.36.

141. A cocrystal according to embodiment 58, characterized in that the coformer is S-proline and the X-ray powder diffraction pattern comprises at least three peak positions, in degrees 2-theta (±0.2 degrees 2-theta), selected from the group consisting of 5.91, 7.19, 14.02 and 26.36.

142. A cocrystal according to embodiment 58, characterized in that the coformer is S-proline and the X-ray powder diffraction pattern comprises the following four peak positions, in degrees 2-theta (±0.2 degrees 2-theta), selected from the group consisting of 5.91, 7.19, 14.02 and 26.36.

143. A cocrystal according to embodiment 58, characterized in that the coformer is S-proline and the X-ray powder diffraction pattern comprises peak positions, in degrees 2-theta (±0.2 degrees 2-theta), of 5.91 and 7.19, and at least one peak position selected from the group consisting of 14.02 and 26.36.

144. A cocrystal according to embodiment 58, characterized in that the coformer is pyruvic acid and the X-ray powder diffraction pattern comprises at least two peak positions, in degrees 2-theta (±0.2 degrees 2-theta), selected from the group consisting of 6.61, 7.81, 14.40, 18.99, 26.41 and 27.10.

145. A cocrystal according to embodiment 58, characterized in that the coformer is pyruvic acid and the X-ray powder diffraction pattern comprises at least three peak positions, in degrees 2-theta (±0.2 degrees 2-theta), selected from the group consisting of 6.61, 7.81, 14.40, 18.99, 26.41 and 27.10.

146. A cocrystal according to embodiment 58, characterized in that the coformer is pyruvic acid and the X-ray powder diffraction pattern comprises at least four peak positions, in degrees 2-theta (±0.2 degrees 2-theta), selected from the group consisting of 6.61, 7.81, 14.40, 18.99, 26.41 and 27.10.

147. A cocrystal according to embodiment 58, characterized in that the coformer is pyruvic acid and the X-ray powder diffraction pattern comprises peak positions, in degrees 2-theta (±0.2 degrees 2-theta), of 6.61 and 7.81, and at least one peak position selected from the group consisting of 14.40, 18.99, 26.41 and 27.10.

148. A pharmaceutical composition comprising a cocrystal according to any one of embodiments 58-147 and one or more pharmaceutical excipients.

149. The pharmaceutical composition of embodiment 148, characterized in that the pharmaceutical composition comprises 1-10% w/w of the compound of formula (I).

150. The pharmaceutical composition of embodiment 148 or 149, characterized in that the pharmaceutical composition is in the form of an orally acceptable dosage form and comprises about 10 mg, about 20 mg, about 25 mg, about 40 mg, about 50 mg, about 100 mg, about 200 mg, or about 300 mg of the compound of formula (I).

151. The pharmaceutical composition of embodiment 150, characterized in that the pharmaceutical composition comprises about 10 mg or about 40 mg of the compound of formula (I).

152. The pharmaceutical composition of embodiment 148, characterized in that the pharmaceutical composition comprises 20-30% w/w of the compound of formula (I).

153. The pharmaceutical composition of embodiment 152, characterized in that the pharmaceutical composition is in the form of an orally acceptable dosage form and comprises about 10 mg, about 20 mg, about 25 mg, about 40 mg, about 50 mg, about 100 mg, about 200 mg, or about 300 mg of the compound of formula (I).

154. An amorphous solid dispersion prepared from a cocrystal according to anyone of embodiments 58 to 147 and a polymer.

155. An amorphous solid dispersion prepared from a cocrystal according to embodiment 154, characterized in that the polymer is selected from the group consisting of hydroxypropyl methyl cellulose (HPMC), hydroxypropyl methylcellulose acetate succinate (HPMCAS), hydroxypropyl methyl cellulose phthalate (HPMCP), hydroxypropyl cellulose (HPC), ethylcellulose, cellulose acetate phthalate, and polyvinylpyrrolidone (PVP), or a mixture thereof.

156. An amorphous solid dispersion prepared from a cocrystal according to embodiment 155, characterized in that the polymer is HPMCAS.

157. A pharmaceutical composition comprising a solid dispersion according to any one of embodiments 154 to 156 and one or more acceptable excipients.

158. A process for the preparation of a pharmaceutically acceptable salt according to embodiment 1 comprising:
dissolving the compound of formula (I) and a pharmaceutically acceptable acid in a solvent to afford a solution; and
precipitating the corresponding salt.

159. A process for the preparation of a crystalline pharmaceutically acceptable salt according to embodiment 2 comprising:
dissolving the compound of formula (I) and a pharmaceutically acceptable acid in a solvent to afford a solution; and
precipitating the corresponding salt in a crystalline form.

160. A process according to embodiment 159, characterized in that the solution is seeded.

161. A process according to any one of embodiments 158 to 160, characterized in that the pharmaceutically acceptable acid is selected from the group consisting of benzene sulfonic acid, (+)-camphor-10-sulfonic acid, ethane-1,2-disulfonic acid, ethanesulfonic acid, hydrobromic acid, hydrochloric acid, naphthalene-1,5-disulfonic acid, naphthalene-2-sulfonic acid, p-toluenesulfonic acid, sulfuric acid and ortho phosphoric acid.

162. A process according to any one of embodiments 158 to 161, characterized in that the solvent is a polar organic solvent selected from the group consisting of methanol, ethyl acetate, acetonitrile, acetone, tetrahydrofurane (THF), or n-butanol.

163. A process according to any one of embodiments 158 to 161, characterized in that the solvent is a mixture of acetone and water.

164. A process for the preparation of a cocrystal of the compound of formula (I) according to embodiment 58, comprising:
dissolving the compound of formula (I) and a coformer in a solvent to afford a solution; and
precipitating the corresponding cocrystal.

165. A process according to embodiment 164, characterized in that the solution is seeded.

166. A process according to any one of embodiments 163 to 165, characterized in that the coformer is selected from a group consisting of 3-hydroxy-2-naphtoic acid, L-serine, glycine, D-gluconic acid, glycolic acid, L-malic acid, oxalic acid, benzoic acid, fumaric acid, gentisic acid, glutaric acid, 4-hydroxybenzoic acid, alpha-ketoglutaric acid, malonic acid, salicylic acid, L-tartaric acid, urea, pyroglutamic acid, caproic acid, glycerol, L-lysine, S-proline and pyruvic acid.

167. A process according to any one of embodiments 163 to 166, characterized in that the solvent comprises a polar organic solvent, selected from a group consisting of methanol, ethyl acetate, acetonitrile, acetone, tetrahydrofurane (THF), or n-butanol.

168. A process according to any one of embodiments 163 to 166, characterized in that the solvent is a mixture of acetone and water.

169. A process according to any one of embodiments 163 to 166, characterized in that the solvent comprises ethyl acetate or methanol.

170. A process for the preparation of an amorphous solid dispersion according to any one of embodiments 54 to 56 or 154 to 156.

171. A pharmaceutically acceptable salt according to anyone of embodiments 1-47 or a pharmaceutical composition according to any one of embodiments 48 to 53 or 57 or an amorphous solid dispersion according to any one of embodiments 54 to 56 for use in the treatment of a cancer characterized by the presence of an IDH1 and/or IDH2 mutation.

172. The pharmaceutically acceptable salt, pharmaceutical composition or amorphous solid dispersion for use according to embodiment 171, characterized in that the cancer is characterized by the presence of an IDH1 mutation.

173. The pharmaceutically acceptable salt, pharmaceutical composition or amorphous solid dispersion for use according to embodiment 172, characterized in that the IDH1 mutation is an R132X mutation.

174. The pharmaceutically acceptable salt, pharmaceutical composition or amorphous solid dispersion for use according to embodiments 173, characterized in that the IDH1 mutation is an R132H, R132C, R132S, R132L or R132G mutation.

175. The pharmaceutically acceptable salt, pharmaceutical composition or amorphous solid dispersion for use according to any one of embodiments 172 to 125, characterized in that the IDH1 mutation results in accumulation of R(−)-2-hydroxyglutarate in the patient.

176. The pharmaceutically acceptable salt, pharmaceutical composition or amorphous solid dispersion for use according to embodiment 171, characterized in that the cancer is characterized by the presence of an IDH2 mutation.

177. The pharmaceutically acceptable salt for use according to embodiment 176, characterized in that the IDH2 mutation is an R140X mutation.

178. The pharmaceutically acceptable salt, pharmaceutical composition or amorphous solid dispersion for use according to embodiment 177, characterized in that the IDH2 mutation is an R140Q, R140W, or R140L mutation.

179. The pharmaceutically acceptable salt, pharmaceutical composition or amorphous solid dispersion for use according to embodiment 176, characterized in that the IDH2 mutation is an R172X mutation.

180. The pharmaceutically acceptable salt, pharmaceutical composition or amorphous solid dispersion for use according to embodiment 179, characterized in that the IDH2 mutation is an R172K or R172G mutation.

181. The pharmaceutically acceptable salt, pharmaceutical composition or amorphous solid dispersion for use according to any one of embodiments 176 to 180, characterized in that the IDH2 mutation results in accumulation of R(−)-2-hydroxyglutarate in the patient.

182. The pharmaceutically acceptable salt, pharmaceutical composition or amorphous solid dispersion for use according to any one of embodiments 171 to 181, characterized in that the cancer is characterized by the presence of an IDH1 mutation and an IDH2 mutation.

183. The pharmaceutically acceptable salt, pharmaceutical composition or amorphous solid dispersion for use according to embodiment 182, characterized in that the IDH1 and IDH2 mutations collectively result in accumulation of R(−)-2-hydroxyglutarate in a patient.

184. The pharmaceutically acceptable salt, pharmaceutical composition or amorphous solid dispersion for use according to any one of embodiments 171 to 183, characterized in that the cancer is a brain tumor.

185. The pharmaceutically acceptable salt, pharmaceutical composition or amorphous solid dispersion for use according to embodiment 184, characterized in that the brain tumor is glioma.

186. The pharmaceutically acceptable salt, pharmaceutical composition or amorphous solid dispersion for use according to embodiment 185, characterized in that the glioma is a low grade glioma or a secondary high grade glioma.

187. The pharmaceutically acceptable salt, pharmaceutical composition or amorphous solid dispersion for use according to embodiment 185 or 186, characterized in that the glioma is a secondary high grade glioma, and the secondary high grade glioma is glioblastoma.

188. The pharmaceutically acceptable salt, pharmaceutical composition or amorphous solid dispersion for use according to any one of embodiments 171 to 187, characterized in that the cancer is refractory or relapsed.

189. The pharmaceutically acceptable salt, pharmaceutical composition or amorphous solid dispersion for use according to any one of embodiments 171 to 188, characterized in that the cancer is newly diagnosed or previously untreated.

190. The pharmaceutically acceptable salt, pharmaceutical composition or amorphous solid dispersion for use according to any one of embodiments 171 to 189, characterized in that the pharmaceutically acceptable salt is administered with an additional therapy.

191. The pharmaceutically acceptable salt, pharmaceutical composition or amorphous solid dispersion for use according to any one of embodiments 171 to 190, characterized in that the pharmaceutically acceptable salt is administered in an amount of about 10 mg, about 20 mg, about 25 mg, about 40 mg, about 50 mg, about 100 mg, about 200 mg, or about 300 mg per day, based on the amount of the compound of formula (I).

192. The pharmaceutically acceptable salt, pharmaceutical composition or amorphous solid dispersion for use according to any one of embodiments 171 to 190, characterized in that the pharmaceutically acceptable salt is administered in an amount of about 10 mg, about 20 mg or about 40 mg per day, based on the amount of the compound of formula (I).

193. The pharmaceutically acceptable salt, pharmaceutical composition or amorphous solid dispersion for use according to any one of embodiments 171 to 190, characterized in that the pharmaceutically acceptable salt is administered in an amount of about 10 mg, about 20 mg, about 25 mg, about 40 mg, about 50 mg, about 100 mg, about 200 mg, or about 300 mg, twice per day, based on the amount of the compound of formula (I).

194. The pharmaceutically acceptable salt, pharmaceutical composition or amorphous solid dispersion for use according to any one of embodiments 171 to 190, characterized in that the pharmaceutically acceptable salt is administered in an amount of about 10 mg, about 20 mg or about 40 mg, twice per day, based on the amount of the compound of formula (I).

195. A cocrystal according to anyone of embodiments 58 to 147 or a pharmaceutical composition according to any one of embodiments 148 to 153 or 157 or an amorphous solid dispersion according to any one of embodiments 154 to 156 for use in the treatment of a cancer characterized by the presence of an IDH1 and/or IDH2 mutation.

196. The cocrystal or pharmaceutical composition or amorphous solid dispersion for use according to embodiment 195, characterized in that the cancer is characterized by the presence of an IDH1 mutation.

197. The cocrystal or pharmaceutical composition or amorphous solid dispersion for use according to embodiment 196, characterized in that the IDH1 mutation is an R132X mutation.

198. The cocrystal or pharmaceutical composition or amorphous solid dispersion for use according to embodiments 197, characterized in that the IDH1 mutation is an R132H, R132C, R132S, R132L or R132G mutation.

199. The cocrystal or pharmaceutical composition or amorphous solid dispersion for use according to any one of embodiments 196 to 198, characterized in that the IDH1 mutation results in accumulation of R(−)-2-hydroxyglutarate in the patient.

200. The cocrystal or pharmaceutical composition or amorphous solid dispersion for use according to embodiment 195, characterized in that the cancer is characterized by the presence of an IDH2 mutation.

201. The cocrystal or pharmaceutical composition or amorphous solid dispersion for use according to embodiment 200, characterized in that the IDH2 mutation is an R140X mutation.

202. The cocrystal or pharmaceutical composition or amorphous solid dispersion for use according to embodiment 201, characterized in that the IDH2 mutation is an R140Q, R140W, or R140L mutation.

203. The cocrystal or pharmaceutical composition or amorphous solid dispersion for use according to embodiment 200, characterized in that the IDH2 mutation is an R172X mutation.

204. The cocrystal or pharmaceutical composition or amorphous solid dispersion for use according to embodiment 203, characterized in that the IDH2 mutation is an R172K or R172G mutation.

205. The cocrystal or pharmaceutical composition or amorphous solid dispersion for use according to any one of embodiments 200 to 204, characterized in that the IDH2 mutation results in accumulation of R(−)-2-hydroxyglutarate in the patient.

206. The cocrystal for use according to any one of embodiments 195 to 205, characterized in that the cancer is characterized by the presence of an IDH1 mutation and an IDH2 mutation.

207. The cocrystal or pharmaceutical composition or amorphous solid dispersion for use according to embodiment 206, characterized in that the IDH1 and IDH2 mutations collectively result in accumulation of R(−)-2-hydroxyglutarate in a patient.

208. The cocrystal or pharmaceutical composition or amorphous solid dispersion for use according to any one of embodiments 195 to 207, characterized in that the cancer is a brain tumor.

209. The cocrystal or pharmaceutical composition or amorphous solid dispersion for use according to embodiment 208, characterized in that the brain tumor is glioma.

210. The cocrystal or pharmaceutical composition or amorphous solid dispersion for use according to embodiment 209, characterized in that the glioma is a low grade glioma or a secondary high grade glioma.

211. The cocrystal or pharmaceutical composition or amorphous solid dispersion for use according to embodiment 209 or 210, characterized in that the glioma is a secondary high grade glioma, and the secondary high grade glioma is glioblastoma.

212. The cocrystal or pharmaceutical composition or amorphous solid dispersion for use according to any one of embodiments 195 to 211, characterized in that the cancer is refractory or relapsed.

213. The cocrystal or pharmaceutical composition or amorphous solid dispersion for use according to any one of embodiments 195 to 212, characterized in that the cancer is newly diagnosed or previously untreated.

214. The cocrystal or pharmaceutical composition or amorphous solid dispersion for use according to any one of embodiments 195 to 213, characterized in that the cocrystal is administered with an additional therapy.

215. The cocrystal or pharmaceutical composition or amorphous solid dispersion for use according to any one of embodiments 195 to 214, characterized in that the cocrystal is administered in an amount of about 10 mg, about 20 mg, about 25 mg, about 40 mg, about 50 mg, about 100 mg, about 200 mg, or about 300 mg per day, based on the amount of the compound of formula (I).

216. The cocrystal or pharmaceutical composition or amorphous solid dispersion for use according to any one of embodiments 195 to 214, characterized in that cocrystal is administered in an amount of about 10 mg, about 20 mg or about 40 mg per day, based on the amount of the compound of formula (I).

217. The cocrystal or pharmaceutical composition or amorphous solid dispersion for use according to any one of embodiments 195 to 214, characterized in that the cocrystal is administered in an amount of about 10 mg, about 20 mg, about 25 mg, about 40 mg, about 50 mg, about 100 mg, about 200 mg, or about 300 mg, twice per day, based on the amount of the compound of formula (I).

218. The cocrystal or pharmaceutical composition or amorphous solid dispersion for use according to any one of embodiments 195 to 214, characterized in that the cocrystal is administered in an amount of about 10 mg, about 20 mg or about 40 mg, twice per day, based on the amount of the compound of formula (I).

219. A pharmaceutically acceptable salt according to embodiment 1, characterized in that the pharmaceutically acceptable salt is formed using benzene sulfonic acid.

220. A pharmaceutically acceptable salt according to embodiment 1, characterized in that the pharmaceutically acceptable salt is formed using (+)-camphor-10-sulfonic acid.

221. A pharmaceutically acceptable salt according to embodiment 1, characterized in that the pharmaceutically acceptable salt is formed using ethane-1,2-disulfonic acid.

222. A pharmaceutically acceptable salt according to embodiment 1, characterized in that the pharmaceutically acceptable salt is formed using ethanesulfonic acid.

223. A pharmaceutically acceptable salt according to embodiment 1, characterized in that the pharmaceutically acceptable salt is formed using hydrobromic acid.

224. A pharmaceutically acceptable salt according to embodiment 1, characterized in that the pharmaceutically acceptable salt is formed using hydrochloric acid.

225. A pharmaceutically acceptable salt according to embodiment 1, characterized in that the pharmaceutically acceptable salt is formed using naphthalene-1,5-disulfonic acid.

226. A pharmaceutically acceptable salt according to embodiment 1, characterized in that the pharmaceutically acceptable salt is formed using naphthalene-2-sulfonic acid.

227. A pharmaceutically acceptable salt according to embodiment 1, characterized in that the pharmaceutically acceptable salt is formed using p-toluenesulfonic acid.

228. A pharmaceutically acceptable salt according to embodiment 1, characterized in that the pharmaceutically acceptable salt is formed using sulfuric acid.

229. A pharmaceutically acceptable salt according to embodiment 1, characterized in that the pharmaceutically acceptable salt is formed using ortho phosphoric acid.

230. A cocrystal according to embodiment 58, characterized in that the coformer is 3-hydroxy-2-naphtoic acid.

231. A cocrystal according to embodiment 58, characterized in that the coformer is L-serine.

232. A cocrystal according to embodiment 58, characterized in that the coformer is glycine.

233. A cocrystal according to embodiment 58, characterized in that the coformer is D-gluconic acid.

234. A cocrystal according to embodiment 58, characterized in that the coformer is glycolic acid.

235. A cocrystal according to embodiment 58, characterized in that the coformer is L-malic acid.

236. A cocrystal according to embodiment 58, characterized in that the coformer is oxalic acid.

237. A cocrystal according to embodiment 58, characterized in that the coformer is benzoic acid.

238. A cocrystal according to embodiment 58, characterized in that the coformer is fumaric acid.

239. A cocrystal according to embodiment 58, characterized in that the coformer is gentisic acid.

240. A cocrystal according to embodiment 58, characterized in that the coformer is glutaric acid.

241. A cocrystal according to embodiment 58, characterized in that the coformer is 4-hydroxybenzoic acid.

242. A cocrystal according to embodiment 58, characterized in that the coformer is alpha-ketoglutaric acid.

243. A cocrystal according to embodiment 58, characterized in that the coformer is malonic acid.

244. A cocrystal according to embodiment 57, characterized in that the coformer is salicylic acid.

245. A cocrystal according to embodiment 58, characterized in that the coformer is L-tartaric acid.

246. A cocrystal according to embodiment 58, characterized in that the coformer is urea.

247. A cocrystal according to embodiment 58, characterized in that the coformer is pyroglutamic acid.

248. A cocrystal according to embodiment 58, characterized in that the coformer is caproic acid.

249. A cocrystal according to embodiment 58, characterized in that the coformer is glycerol.

250. A cocrystal according to embodiment 58, characterized in that the coformer is L-lysine.

251. A cocrystal according to embodiment 58, characterized in that the coformer is S-proline.

252. A cocrystal according to embodiment 58, characterized in that the coformer is pyruvic acid.

### EXAMPLES

### Lexicon

TABLE 2

| Abbrevation | Meaning |
|---|---|
| s | singulet |
| d | doublet |
| bd | broad doublet |
| bdₛ | broad doublets |
| t | triplet |
| m | multiplet |
| bm | broad multiplet |
| qa | quadruplet |
| qi | quintuplet |
| vbs | very broad singulet |

### General Experimental Notes

In the following examples, except where otherwise noted, the reagents (chemicals) were purchased from commercial sources (such as Alfa, Acros, Sigma Aldrich, TCI and Shanghai Chemical Reagent Company), and used without further purification.

### Instrumentation and Methods

### X-Ray Powder Diffraction (XRPD) Analysis

The XRPD patterns were recorded from 3.5° 2q to 35° 2q using an Empyrean diffractometer from Panalytical operating in the transmission mode with CuKα radiation (I=1.5418 Å) at 45 kV and 40 mA and with a 0.013 ° 2q step size for 15 minutes (examples 1 to 6, 8, 10 and 13) The XRPD patterns were recorded from 3.5 ° 2θ to 55° 2θ using an Empyrean diffractometer from Panalytical diffractometer operating in the transmission mode with CuKα radiation (I=1.5418 Å) at 45 kV and 40 mA and with a 0.013 ° 2q step size for 30 minutes (examples 7, 9, 11, 12 and 14 to 34).

Only peaks with a relative intensity of above 10% were described in Tables 3-36.

### ¹H NMR Analysis

¹H Liquid NMR spectra were collected on a Bruker 400 MHz NMR Spectrometer.

The chemical shifts, in ppm, are given with respect to tetramethylsilane (TMS), using partially deuterated dimethylsulfoxyde or partially deuterated methanol as internal standard.

In solution in partially deuterated dimethylsulfoxyde, the resonance at 2.5 ppm on the 1D ¹H NMR spectrum is due to partially deuterated dimethylsulfoxyde and the resonance at 3.30 ppm is due to the presence of water.

In solution in partially deuterated methanol, the resonance at 3.3 ppm on the 1D ¹H NMR spectrum is due to partially deuterated methanol and the resonance at 4.83 ppm is due to the presence of water.

### DSC/TGA Analysis

The DSC (Differential Scanning calorimetry) analyses were recorded from 0° C. to 200° C., 250° C. or 350° C. (depending on the nature of the crystalline phase) at 10° C./min using a DSC Q2000 from TA instrument for crystalline phase characterization. For the anhydrous Tg measurement, a first cycle from 25° C. to 110° C. at 10° C./min was performed to remove water, then a second cycle from 110° C. to 20° C. to obtain a fresh glass and finally a third cycle from 20° C. to 200° C. at 1° C./min with modulation of +/−0.32° C. every 60 seconds to determine the Tg value.

The TG (Thermogravimetric) analyses were recorded from 25° C. to 200° C., 250° C. or 350° C. (depending on the nature of the crystalline phase) at 10° C./min using a TGA Q5000 from TA instrument.

In the Examples, it is referred to compound of formula (I) that is 6-(6-chloropyridin-2-yl)-N²,N⁴-bis((R)-1,1,1-trifluoropropan-2-yl)-1,3,5-triazine-2,4-diamine, as Compound 1.

As used in the Examples, the term “Compound 1” shall be understood to refer to 6-(6-chloropyridin-2-yl)-N²,N⁴-bis((R)-1,1,1-trifluoropropan-2-yl)-1,3,5-triazine-2,4-diamine or any tautomer(s) or rotamer(s) thereof. The double bond geometries of the foregoing tautomers were not determined, and therefore the chemical structures representing the foregoing tautomers are not intended to imply a particular double bond geometry.

As used in the Examples, the term “free form” refers to the crystalline form A of Compound 1, which can be prepared according to the process detailed in Examples 10 and 16 of PCT application WO2019/090059.

### Example 1: Preparation and Characterization of Crystalline Benzenesulfonic Acid Salt of Compound 1

Compound 1 (200 mg) and benzenesulfonic acid (1 eq.) were placed in acetone (1.7 mL) and then water (0.8 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

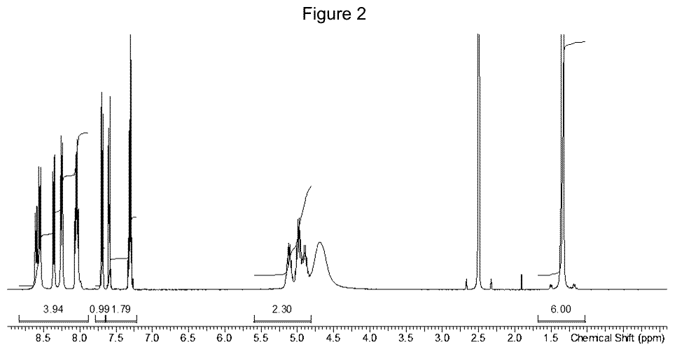

The ¹H NMR spectrum of the benzenesulfonic acid salt of Compound 1 is shown in FIG. 2.

¹H NMR (DMSO-d⁶) δ 8.60 ppm/8.55 ppm/8.36 ppm/8.25 ppm (3H, bdₛ), 8.05 ppm (1H, m) 7.69 ppm (1H, m), 7.59 ppm (1H, m), 7.35-7.26 ppm (1H, m), 5.20-4.83 ppm (2H, bm), 1.34 ppm (6H, bd).

The XRPD pattern of the salt is shown in FIG. 1. The peak positions, peak heights, and relative intensities of the peaks in the XRPD pattern are listed in Table 3.

TABLE 3
XRPD Peaks of benzenesulfonic acid salt of Compound 1

| Position | Intensity | Relative |
|---|---|---|
| [° 2 Theta] | [counts] | Intensity [%] |
| 4.08 | 3295 | 18.3 |
| 5.83 | 9391 | 100.0 |
| 6.84 | 2871 | 16.6 |
| 8.17 | 6927 | 69.5 |
| 8.42 | 2712 | 13.6 |
| 11.71 | 2370 | 11.3 |
| 12.28 | 2639 | 14.5 |
| 13.15 | 2947 | 17.7 |
| 14.14 | 4488 | 37.1 |
| 14.40 | 8999 | 94.9 |
| 15.60 | 4066 | 30.9 |
| 16.27 | 3086 | 17.8 |
| 16.46 | 2648 | 12.0 |
| 17.65 | 4424 | 33.5 |
| 17.90 | 6409 | 57.2 |
| 18.89 | 2823 | 11.3 |
| 19.35 | 5059 | 39.2 |
| 19.87 | 2919 | 12.3 |
| 20.65 | 3350 | 16.6 |
| 20.98 | 3818 | 23.1 |
| 23.54 | 3126 | 12.1 |
| 24.27 | 3474 | 14.6 |
| 26.24 | 4843 | 30.0 |
| 26.50 | 5351 | 36.5 |
| 26.78 | 5577 | 38.9 |
| 27.13 | 6566 | 52.1 |
| 27.57 | 4452 | 24.3 |
| 28.22 | 3791 | 16.8 |
| 28.81 | 4097 | 21.3 |
| 29.16 | 3212 | 10.9 |

### Example 2: Preparation and Characterization of Crystalline (+)-Camphor-10-Sulfonic Acid Salt of Compound 1

Compound 1 (200 mg) and (+)-camphor-10-sulfonic acid (1 eq.) and were placed in acetone (1 mL) and then water (0.5 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

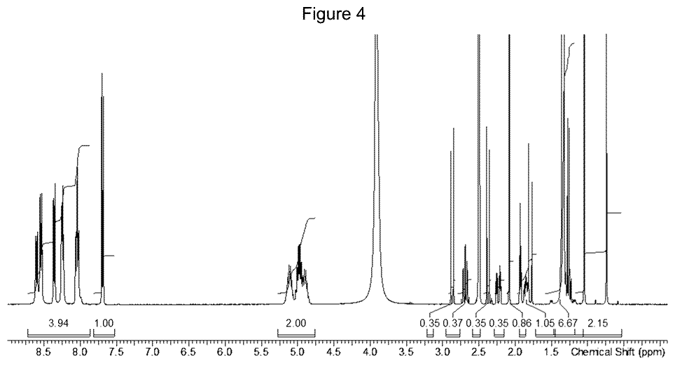

The ¹H NMR spectrum of the (+)-camphor-10-sulfonic acid salt of compound 1 is shown in FIG. 4.

¹H NMR (DMSO-d₆) δ 8.60 ppm/8.54 ppm/8.36 ppm/8.25 ppm and 8.24 ppm (3H, bdₛ), 8.04 ppm (1H, m) 7.69 ppm (1H, bd), 5.20-4.82 ppm (2H, bm), 2.87 and 2.38 ppm (1H, dd), 2.69 ppm (1H, bqi), 2.23 ppm (1H, bm), 2.08 ppm (1H, s), 1.93 ppm (1H, bt), 1.85 ppm (1H, m), 1.79 ppm (1H, d), 1.34 ppm (6H, bd), 1.27 ppm (1H, m), 1.05 ppm (1H, s), 0.74 ppm (1H, s).

The XRPD pattern of the (+)-camphor-10-sulfonic acid salt of Compound 1 is shown in FIG. 3. The peak positions, peak heights, and relative intensities of the peaks in the XRPD pattern are listed in Table 4.

TABLE 4
XRPD Peaks of (+)-camphor-10-sulfonic acid
salt of Compound 1

| Position | Intensity | Relative |
|---|---|---|
| [° 2 Theta] | [counts] | Intensity [%] |
| 6.02 | 5915 | 28.0 |
| 7.73 | 4177 | 11.6 |
| 8.29 | 6388 | 46.2 |
| 8.75 | 5348 | 33.3 |
| 9.80 | 4568 | 25.9 |
| 9.93 | 3462 | 10.3 |
| 10.19 | 9634 | 100.0 |
| 10.38 | 3674 | 14.5 |
| 11.63 | 3229 | 11.8 |
| 11.92 | 4169 | 26.3 |
| 12.07 | 8267 | 85.6 |
| 12.85 | 3734 | 21.2 |
| 13.88 | 4211 | 31.1 |
| 14.35 | 5029 | 43.4 |
| 14.62 | 3596 | 23.2 |
| 15.68 | 3661 | 25.8 |
| 15.82 | 2686 | 12.1 |
| 16.13 | 3907 | 30.1 |
| 16.64 | 4589 | 41.0 |
| 16.96 | 4244 | 36.7 |
| 17.40 | 3253 | 23.1 |
| 17.63 | 3782 | 31.2 |
| 18.12 | 4772 | 46.6 |
| 18.82 | 2467 | 14.3 |
| 19.12 | 3585 | 30.0 |
| 19.68 | 4130 | 39.6 |
| 19.97 | 5468 | 59.4 |
| 20.46 | 2807 | 20.8 |
| 21.20 | 2228 | 14.0 |
| 21.71 | 2728 | 22.1 |
| 22.42 | 2288 | 16.5 |
| 24.29 | 1773 | 10.5 |
| 24.66 | 2132 | 16.2 |
| 24.97 | 2556 | 22.7 |
| 25.98 | 1758 | 12.1 |
| 27.00 | 1541 | 10.1 |

### Example 3; Preparation and Characterization of Crystalline Ethane-1.2-Disulfonic Acid Salt of Compound 1

Compound 1 (200 mg) and ethane-1,2-disulfonic acid (0.5 eq.) were placed in acetone (2.4 mL) and then water (1.2 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

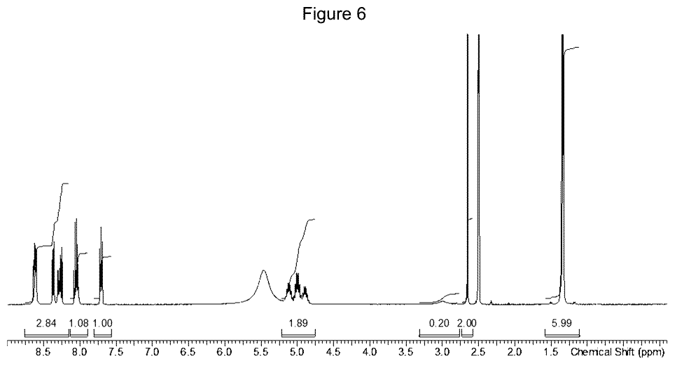

The ¹H NMR spectrum of the ethane-1,2-disulfonic acid salt of Compound 1 is shown in FIG. 6.

¹H NMR (DMSO-d⁶) δ 8.63 ppm/8.61 ppm/8.37 ppm/8.29 ppm and 8.26 ppm (3H, bdₛ), 8.0 ppm (1H, bm) 7.71 ppm (1H, bm), 5.20-4.82 ppm (2H, bm), 2.65 ppm (4H, s), 1.34 ppm (6H, bd).

The XRPD pattern of the salt is shown in FIG. 5. The peak positions, peak heights, and relative intensities of the peaks in the XRPD pattern are listed in Table 5.

TABLE 5
XRPD Peaks of ethane-1,2-disulfonic acid salt of Compound 1

| Position | | Relative |
|---|---|---|
| [° 2 Theta] | Height [counts] | Intensity [%] |
| 5.59 | 18374 | 64.5 |
| 11.05 | 6981 | 19.2 |
| 11.52 | 5039 | 10.4 |
| 14.75 | 15188 | 59.3 |
| 15.14 | 6242 | 17.6 |
| 16.40 | 6307 | 18.6 |
| 16.82 | 7298 | 23.4 |
| 17.46 | 6676 | 20.8 |
| 18.08 | 12048 | 46.1 |
| 18.87 | 23526 | 100.0 |
| 21.10 | 10890 | 41.8 |
| 22.50 | 8441 | 30.8 |
| 22.99 | 4547 | 12.9 |
| 23.18 | 6084 | 20.2 |
| 23.51 | 6078 | 20.0 |
| 23.74 | 9800 | 37.6 |
| 24.83 | 6634 | 23.3 |
| 25.78 | 4435 | 13.2 |
| 29.75 | 3958 | 13.6 |
| 30.59 | 4989 | 18.5 |

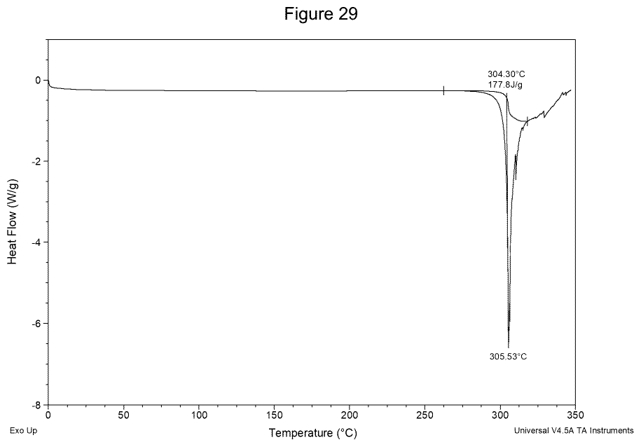

The DSC thermogram of the ethane-1,2-disulfonic acid salt of Compound 1 depicted FIG. 29 comprises an endothermic peak having an onset temperature of 304.30° C. and peak temperature of 305.53° C.

### Example 4: Preparation and Characterization of Crystalline Ethanesulfonic Acid Salt of Compound 1

Compound 1 (50 mg) and ethanesulfonic acid (0.5 eq.) were placed in acetone (0.8 mL) and then water (0.4 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

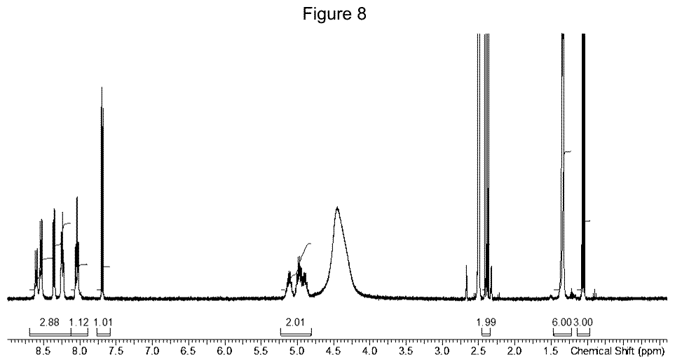

The ¹H NMR spectrum of the ethanesulfonic acid salt of Compound 1 is shown in FIG. 8. ¹H NMR (DMSO-d⁶) δ 8.60 ppm/8.54 ppm/8.36 ppm and 8.25 ppm 8.24 ppm (3H, bdₛ), 8.04 ppm (1H, m) 7.69 ppm (1H, d), 5.20-4.83 ppm (2H, bm), 2.39 ppm (2H, qa), 1.34 ppm (6H, bd), 1.06 ppm (3H, t)

The XRPD pattern of the salt is shown in FIG. 7. The peak positions, peak heights, and relative intensities of the peaks in the XRPD pattern are listed in Table 6.

TABLE 6
XRPD Peaks of ethanesulfonic acid salt of Compound 1

| Position | Intensity | |
|---|---|---|
| [° 2 Theta] | [counts] | Relative Intensity [%] |
| 6.86 | 17982 | 19.26 |
| 11.81 | 10913 | 11 |
| 13.97 | 78853 | 100 |
| 14.27 | 29907 | 35.69 |
| 17.00 | 17913 | 20.3 |
| 17.72 | 19808 | 22.71 |
| 20.72 | 11314 | 11.86 |
| 22.68 | 14177 | 15.92 |
| 25.82 | 29317 | 35.85 |
| 26.47 | 10110 | 10.83 |
| 27.08 | 21827 | 26 |
| 28.25 | 11302 | 11.2 |

### Example 5: Preparation and Characterization of Crystalline Hydrobromic Acid Salt of Compound 1

Compound 1 (200 mg) and hydrobromic acid (1 eq. placed in 0.2 ml of water) were placed in acetone (1 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

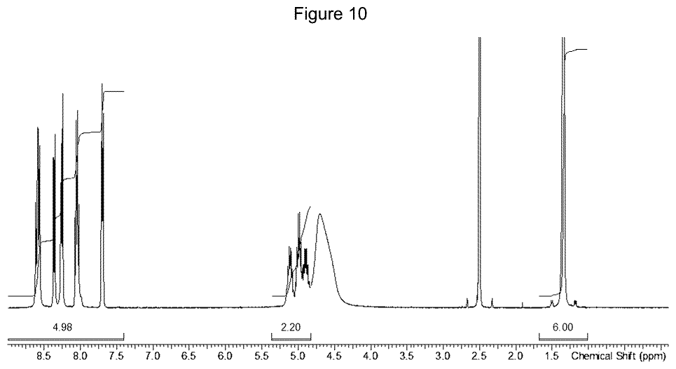

The ¹H NMR spectrum of the hydrobromic acid salt of Compound 1 is shown in FIG. 10. ¹H NMR (DMSO-d⁶) δ 8.61 ppm/8.57 ppm/8.36 ppm and 8.26 ppm (3H, bdₛ), 8.05 ppm (1H, bm) 7.70 ppm (1H, bm), 5.20-4.83 ppm (2H, bm), 1.34 ppm (6H, d).

The XRPD pattern of the salt is shown in FIG. 9. The peak positions, peak heights, and relative intensities of the peaks in the XRPD pattern are listed in Table 7.

TABLE 7
XRPD Peaks of hydrobromic acid salt of Compound 1

| Position | | |
|---|---|---|
| [° 2 Theta] | Height [counts] | Relative Intensity [%] |
| 5.74 | 11463 | 100.0 |
| 8.56 | 10323 | 97.3 |
| 10.20 | 3392 | 11.3 |
| 11.52 | 3716 | 18.4 |
| 13.63 | 5314 | 40.7 |
| 13.96 | 4763 | 34.4 |
| 14.71 | 4438 | 31.1 |
| 14.90 | 6210 | 53.9 |
| 15.08 | 5343 | 42.7 |
| 17.21 | 2857 | 12.9 |
| 18.04 | 3487 | 21.2 |
| 18.29 | 4662 | 37.0 |
| 19.11 | 3324 | 20.7 |
| 20.50 | 4071 | 31.6 |
| 21.01 | 4358 | 35.4 |
| 21.93 | 2636 | 13.8 |
| 22.23 | 2532 | 13.8 |
| 23.16 | 3596 | 26.6 |
| 24.52 | 2389 | 12.3 |
| 26.37 | 6099 | 60.3 |
| 27.01 | 7102 | 73.4 |
| 27.40 | 3596 | 28.1 |
| 28.12 | 3077 | 22.0 |
| 28.78 | 3250 | 23.7 |

### Example 6: Preparation and Characterization of Crystalline Hydrochloric Acid Salt of Compound 1

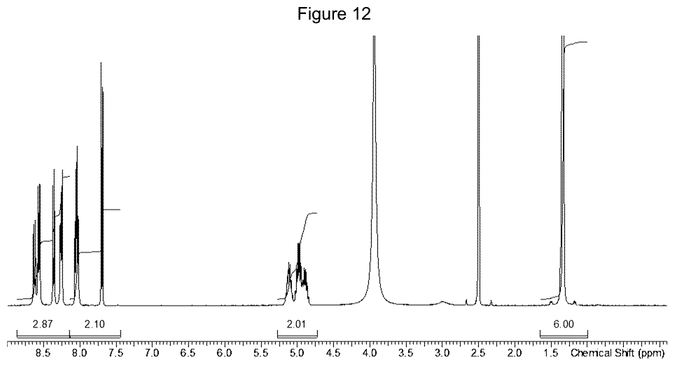

Compound 1 (1 g) and hydrochloric acid (1 eq. placed in 7.5 ml of water) were placed in acetone (10 mL). The mixture was stirred and heated up to 50° C. at 1° C./min, followed by 2 hours at 50° C. and then cooled down to 20° C. at 0.1° C./min. The mixture was then maintained under stirring at 20° C. for 3 days before filtration and drying at 40° C. under vacuum overnight. The ¹H NMR spectrum of the hydrochloric acid salt of Compound 1 is shown in FIG. 12.

¹H NMR (DMSO-d⁶) δ8.63 ppm/8.56 ppm/8.36 ppm/8.26 ppm and 8.25 ppm (3H, bdₛ), 8.05 ppm (1H, bm) 7.70 ppm (1H, bd), 5.20-4.83 ppm (2H, bm), 1.34 ppm (6H, bd). The XRPD pattern of the salt is shown in FIG. 11. The peak positions, peak heights, and relative intensities of the peaks in the XRPD pattern are listed in Table 8.

TABLE 8
XRPD Peaks of hydrochloric
acid salt of Compound 1

| | | Relative |
|---|---|---|
| Position | Height | Intensity |
| [°2 Theta] | [counts] | [%] |
| 5.73 | 7418 | 81.2 |
| 8.73 | 4557 | 35.2 |
| 9.71 | 6587 | 84.0 |
| 11.50 | 4145 | 38.6 |
| 11.79 | 3026 | 14.5 |
| 13.78 | 5052 | 61.5 |
| 14.18 | 3101 | 19.2 |
| 14.64 | 5885 | 80.5 |
| 14.86 | 3609 | 32.1 |
| 15.25 | 2657 | 12.3 |
| 16.25 | 4426 | 50.8 |
| 17.47 | 4152 | 45.5 |
| 17.98 | 2927 | 21.7 |
| 18.50 | 3412 | 31.3 |
| 18.89 | 2398 | 11.1 |
| 19.38 | 3679 | 38.8 |
| 20.77 | 2450 | 15.2 |
| 20.97 | 2853 | 24.2 |
| 21.25 | 2774 | 23.1 |
| 21.43 | 2814 | 24.1 |
| 22.03 | 2305 | 14.6 |
| 23.54 | 2057 | 12.5 |
| 25.98 | 4707 | 71.7 |
| 26.61 | 6028 | 100.0 |
| 27.01 | 2292 | 21.0 |
| 27.80 | 2953 | 36.1 |
| 28.08 | 2103 | 19.9 |
| 28.52 | 1988 | 16.9 |
| 29.00 | 1728 | 12.8 |
| 29.49 | 2040 | 19.4 |

### Example 7: Preparation and Characterization of Crystalline Naphthalene-1,5-Disulfonic Acid Salt of Compound 1

Compound 1 (200 mg) and naphthalene-1,5-disulfonic acid (0.5 eq.) were placed in acetone (2.4 mL) and then water (1.2 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 3 days before filtration and drying at 40° C. under vacuum overnight.

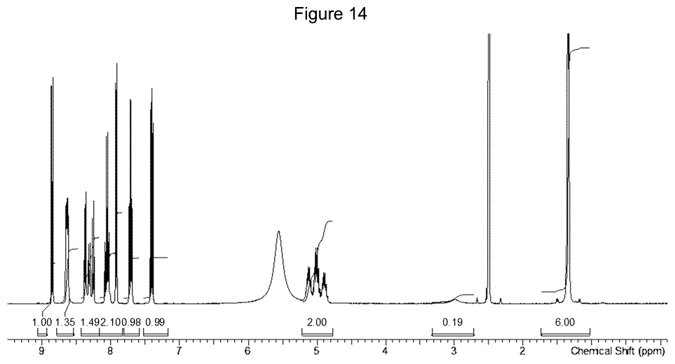

The ¹H NMR spectrum of the naphthalene-1,5-disulfonic acid salt of Compound 1 is shown in FIG. 14.

¹H NMR (DMSO-d⁶) δ 8.85 ppm (1H, d), 8.64 ppm/8.63 ppm/8.37 ppm/8.31 ppm and 8.26 ppm (3H, bdₛ), 8.06 ppm (1H, bm), 7.92 ppm (1H, dd), 7.71 ppm (1H, bt), 7.41 ppm (1H, dd), 5.20-4.82 ppm (2H, bm), 1.34 ppm (6H, bd).

The XRPD pattern of the salt is shown in FIG. 13. The peak positions, peak heights, and relative intensities of the peaks in the XRPD pattern are listed in Table 9.

TABLE 9
XRPD Peaks of naphthalene-1,5-
disulfonic acid salt of Compound 1

| | | Relative |
|---|---|---|
| Position | Height | Intensity |
| [°2 Theta] | [counts] | [%] |
| 5.52 | 40982 | 100.0 |
| 8.04 | 14555 | 22.3 |
| 13.04 | 11238 | 18.4 |
| 13.30 | 9085 | 11.5 |
| 13.79 | 27713 | 70.8 |
| 14.17 | 20107 | 46.9 |
| 14.53 | 13768 | 26.9 |
| 14.77 | 11752 | 20.6 |
| 15.06 | 9649 | 14.0 |
| 15.32 | 10405 | 16.5 |
| 16.14 | 11221 | 19.4 |
| 16.26 | 8618 | 10.5 |
| 17.05 | 9678 | 15.1 |
| 19.28 | 18466 | 44.3 |
| 19.63 | 22570 | 57.6 |
| 19.97 | 32572 | 89.5 |
| 21.14 | 10445 | 20.6 |
| 21.36 | 8973 | 16.0 |
| 22.27 | 9461 | 18.6 |
| 22.84 | 7140 | 11.8 |
| 26.26 | 19507 | 52.5 |
| 26.41 | 11496 | 27.4 |
| 26.86 | 14277 | 36.0 |
| 27.26 | 8811 | 18.9 |
| 27.87 | 6193 | 10.7 |
| 28.22 | 6084 | 10.5 |

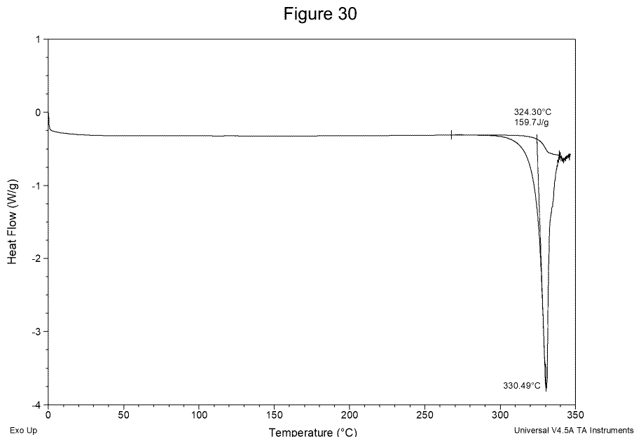

The DSC thermogram of the naphthalene-1,5-disulfonic acid salt depicted in FIG. 30 comprises an endothermic peak having an onset temperature of 324.30° C. and a peak temperature of 330.49° C.

### Example 8: Preparation and Characterization of Crystalline Naphthalene-2-Sulfonic Acid Salt of Compound 1

Compound 1 (50 mg) and naphthalene-2-sulfonic acid (2 eq.) were placed in acetone (0.8 mL) and then water (0.4 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

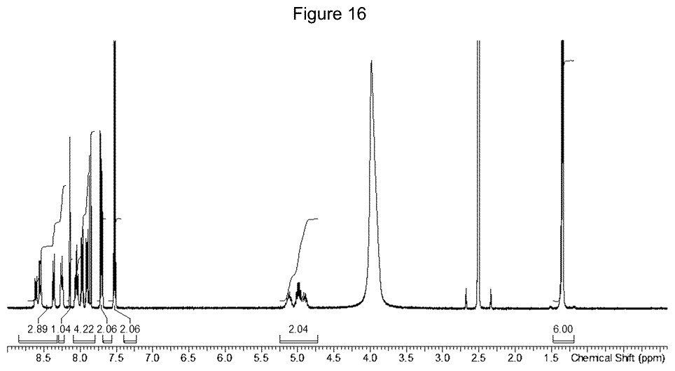

The ¹H NMR spectrum of the naphthalene-2-sulfonic acid salt of Compound 1 is shown in FIG. 16.

¹H NMR (DMSO-d⁶) δ 8.61 ppm/8.55 ppm/8.37 ppm/8.26 ppm and 8.25 ppm (3H, bdₛ), 8.14 (1H, bs), 8.05 ppm (1H, bm), 7.97 ppm and 7.90 ppm (2H, m), 7.86 ppm (1H, bd), 7.73-7.50 ppm (4H, m), 5.20-4.83 ppm (2H, bm), 1.35 ppm (6H, bd).

The XRPD pattern of the salt is shown in FIG. 15. The peak positions, peak heights, and relative intensities of the peaks in the XRPD pattern are listed in Table 10.

TABLE 10
XRPD Peaks of naphthalene-2-
sulfonic acid salt of Compound 1

| | | Relative |
|---|---|---|
| Position | Height | Intensity |
| [°2 Theta] | [counts] | [%] |
| 8.41 | 25852 | 53.0 |
| 8.72 | 8270 | 13.1 |
| 13.65 | 27265 | 57.3 |
| 15.62 | 16221 | 31.6 |
| 16.81 | 7872 | 12.3 |
| 17.51 | 8070 | 12.7 |
| 18.18 | 6975 | 10.2 |
| 20.65 | 9340 | 15.7 |
| 20.95 | 8082 | 12.8 |
| 21.14 | 7253 | 10.8 |
| 21.37 | 9101 | 15.2 |
| 26.09 | 11193 | 19.8 |
| 26.47 | 37638 | 80.3 |
| 26.65 | 46249 | 100.0 |
| 26.97 | 9544 | 16.1 |
| 27.56 | 9018 | 14.9 |
| 28.13 | 7542 | 11.5 |

### Example 9: Preparation and Characterization of Crystalline p-Toluenesulfonic Acid Salt of Compound 1

Compound 1 (1 g) and p-toluenesulfonic acid (2 eq.) were placed in acetone (5 mL) and then water (2.5 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

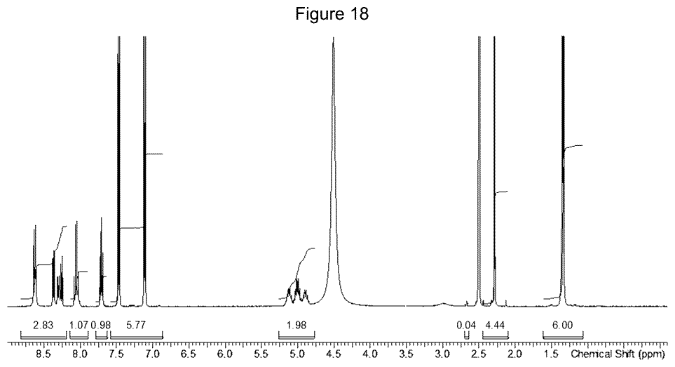

The ¹H NMR spectrum of the p-toluenesulfonic acid salt of Compound 1 is shown in FIG. 18.

¹H NMR (DMSO-d⁶) δ 8.63 ppm/8.37 ppm/8.30 ppm (2H, d) and 8.26 ppm (3H, bdₛ), 8.05 ppm (1H, bm), 7.71 ppm (1H, bt), 7.47 ppm (2H, d), 7.11 ppm (2H, d), 5.20-4.83 ppm (2H, bm), 2.29 ppm (3H, s), 1.34 ppm (6H, bd).

The XRPD pattern of the salt is shown in FIG. 17. The peak positions, peak heights, and relative intensities of the peaks in the XRPD pattern are listed in Table 11.

TABLE 11
XRPD Peaks of p-toluenesulfonic
acid salt of Compound 1

| | | Relative |
|---|---|---|
| Position | Height | Intensity |
| [°2 Theta] | [counts] | [%] |
| 3.91 | 13654 | 45.9 |
| 4.08 | 11047 | 11.6 |
| 4.30 | 12006 | 28.9 |
| 4.76 | 11560 | 29.8 |
| 5.29 | 16169 | 100.0 |
| 5.89 | 11699 | 42.4 |
| 6.02 | 9591 | 13.7 |
| 7.59 | 9518 | 26.0 |
| 7.85 | 9833 | 33.1 |
| 8.16 | 9521 | 31.3 |
| 8.37 | 12624 | 76.3 |
| 8.66 | 11434 | 62.1 |
| 9.32 | 8590 | 26.8 |
| 9.45 | 9126 | 36.0 |
| 11.96 | 8306 | 38.6 |
| 12.96 | 9969 | 60.4 |
| 13.47 | 13207 | 96.5 |
| 14.84 | 7219 | 12.6 |
| 15.10 | 7701 | 19.1 |
| 15.22 | 8424 | 29.2 |
| 15.92 | 7188 | 10.7 |
| 16.26 | 7382 | 13.0 |
| 16.75 | 7391 | 12.0 |
| 17.27 | 7464 | 14.0 |
| 17.65 | 8228 | 24.4 |
| 18.02 | 7933 | 21.6 |
| 18.56 | 7912 | 23.2 |
| 18.69 | 7246 | 14.3 |
| 19.69 | 9048 | 43.6 |
| 20.06 | 9302 | 48.9 |
| 20.25 | 7064 | 18.1 |
| 20.42 | 12548 | 96.2 |
| 20.64 | 10809 | 73.0 |
| 20.91 | 7204 | 24.4 |
| 21.04 | 6941 | 21.3 |
| 21.28 | 8525 | 45.8 |
| 21.46 | 7144 | 26.4 |
| 22.34 | 7513 | 39.9 |
| 25.76 | 10954 | 94.2 |
| 26.06 | 9564 | 74.2 |
| 26.43 | 9525 | 70.9 |
| 26.73 | 7739 | 45.6 |
| 27.25 | 7095 | 38.0 |
| 29.43 | 4000 | 11.3 |

### Example 10: Preparation and Characterization of Crystalline Sulfuric Acid Salt of Compound 1

Compound 1 (1 g) and sulfuric acid (1 eq. placed in 1.5 ml of water) were placed in acetone (5 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

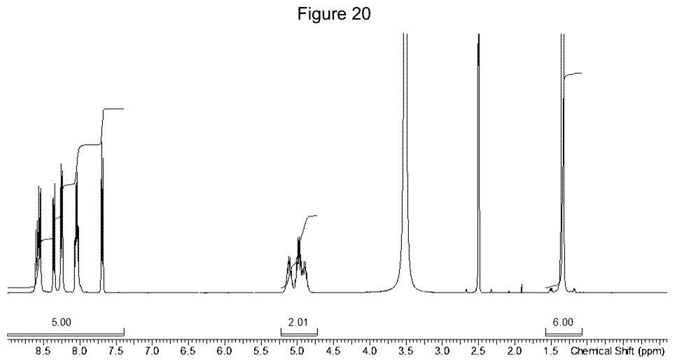

The ¹H NMR spectrum of the sulfuric acid salt of Compound 1 is shown in FIG. 20, respectively.

¹H NMR (DMSO-d₆) δ 8.60 ppm/8.55 ppm/8.36 ppm and 8.25 ppm (3H, bdₛ), 8.04 ppm (1H, bm) 7.69 ppm (1H, bm), 5.20-4.82 ppm (2H, bm), 1.34 ppm (6H, bd).

The XRPD pattern of the salt is shown in FIG. 19. The peak positions, peak heights, and relative intensities of the peaks in the XRPD pattern are listed in Table 12.

TABLE 12
XRPD Peaks of sulfuric acid
salt of Compound 1

| | | Relative |
|---|---|---|
| Position | Height | Intensity |
| [°2 Theta] | [counts] | [%] |
| 5.80 | 15165 | 100.0 |
| 8.50 | 10408 | 65.6 |
| 11.63 | 4277 | 19.2 |
| 13.62 | 9623 | 59.7 |
| 14.21 | 7334 | 42.2 |
| 14.87 | 14256 | 94.9 |
| 17.44 | 3259 | 10.4 |
| 18.04 | 7525 | 42.8 |
| 19.25 | 5765 | 29.2 |
| 20.39 | 4569 | 19.5 |
| 20.97 | 4505 | 19.0 |
| 21.86 | 3973 | 14.8 |
| 22.16 | 3678 | 12.3 |
| 23.36 | 3834 | 13.7 |
| 24.68 | 3451 | 10.5 |
| 25.63 | 3549 | 10.9 |
| 26.10 | 6477 | 33.2 |
| 26.41 | 10511 | 64.3 |
| 26.76 | 8096 | 45.9 |
| 27.08 | 10501 | 63.3 |
| 28.08 | 4852 | 21.0 |
| 28.61 | 5375 | 25.2 |
| 29.03 | 4993 | 22.6 |

### Example 11: Preparation and Characterization of Crystalline 3-Hydroxy-2-Naphtoic Acid Cocrystal of Compound 1

Compound 1 (1 g) and 3-hydroxy-2-naphthoic acid (1 eq) were placed in acetone (10 mL) and then water (2.5 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

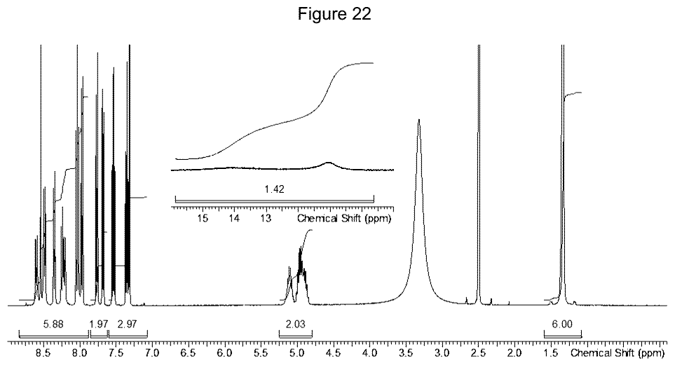

The ¹H NMR spectrum of the 3-hydroxy-2-naphtoic acid cocrystal of Compound 1 is shown in FIG. 22.

¹H NMR (DMSO-d⁶) δ 14.07 ppm (1H, bs), 11.06 ppm (1H, bs), 8.60 ppm/8.49 ppm/8.35 ppm/8.25 ppm and 8.21 ppm (3H, bdₛ), 8.54 ppm (1H, s), 8.04 ppm (1H, t), 7.97 ppm (1H, bd), 7.77 ppm (1H, bd), 7.68 ppm (1H, bd), 7.54 ppm (1H, td), 7.35 ppm (1H, td), 7.32 ppm (1H, s), 5.20-4.82 ppm (2H, bm), 1.34 ppm (6H, bd).

The XRPD pattern of 3-hydroxy-2-naphtoic acid cocrystal of Compound 1 is shown in FIG. 21. The peak positions, peak heights, and relative intensities of the peaks in the XRPD pattern are listed in Table 13.

TABLE 13
XRPD Peaks of 3-hydroxy-2-naphtoic
acid cocrystal of Compound 1

| | | Relative |
|---|---|---|
| Position | Height | Intensity |
| [°2 Theta] | [counts] | [%] |
| 6.41 | 78029 | 83.4 |
| 12.97 | 18625 | 16.0 |
| 13.34 | 65696 | 71.4 |
| 13.69 | 56223 | 59.9 |
| 14.26 | 25737 | 24.7 |
| 15.65 | 21672 | 20.3 |
| 15.84 | 41585 | 43.7 |
| 15.95 | 15764 | 13.5 |
| 16.73 | 25310 | 24.8 |
| 17.11 | 29273 | 29.5 |
| 17.48 | 21475 | 20.4 |
| 18.11 | 17718 | 16.1 |
| 20.21 | 49839 | 54.0 |
| 20.60 | 29807 | 30.7 |
| 23.54 | 14473 | 13.4 |
| 25.59 | 83884 | 95.0 |
| 26.09 | 28312 | 30.0 |
| 26.40 | 87980 | 100.0 |
| 26.84 | 15702 | 15.4 |
| 27.32 | 18096 | 18.2 |
| 30.13 | 18107 | 18.6 |

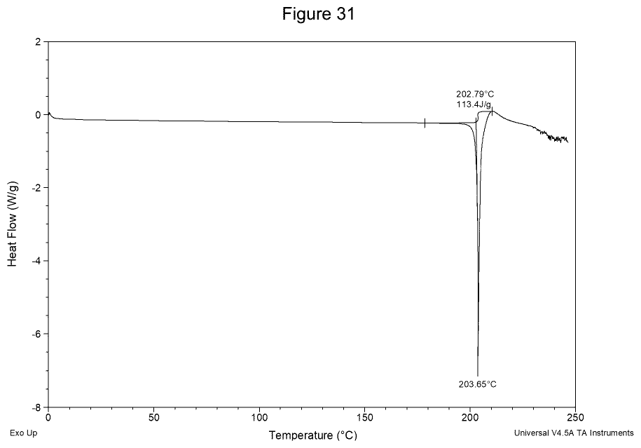

The DSC thermogram of the 3-hydroxy-2-naphtoic acid cocrystal of Compound 1 depicted in FIG. 31 comprises an endothermic peak having an onset temperature of 202.79° C. and peak temperature of 203.65° C.

### Example 12: Preparation and Characterization of Crystalline L-Serine Cocrystal of Compound 1

Compound 1 (200 mg) and L-Serine (0.5 eq) were placed in acetone (2.7 mL) and then water (1.3 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

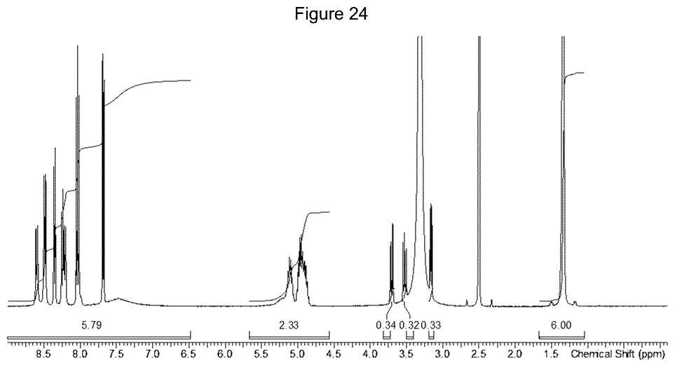

The ¹H NMR spectrum of the L-serine cocrystal of Compound 1 is shown in FIG. 24. ¹H NMR (DMSO-d⁶) δ 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bm), 3.70 ppm (1H, dd), 3.52 ppm (1H, dd), 3.16 ppm (1H, dd), 1.34 ppm (6H, bd).

The XRPD pattern of L-serine cocrystal of Compound 1 is shown in FIG. 23. The peak positions, peak heights, and relative intensities of the peaks in the XRPD pattern are listed in Table 14.

TABLE 14
XRPD Peaks of L-serine
cocrystal of Compound 1

| | | Relative |
|---|---|---|
| Position | Height | Intensity |
| [°2 Theta] | [counts] | [%] |
| 5.90 | 26652 | 53.9 |
| 8.46 | 38905 | 100.0 |
| 13.28 | 26733 | 68.3 |
| 13.83 | 27075 | 68.8 |
| 13.88 | 28929 | 75.4 |
| 15.26 | 10819 | 18.6 |
| 16.22 | 9451 | 14.8 |
| 16.29 | 9727 | 15.6 |
| 16.98 | 11459 | 21.7 |
| 17.46 | 11270 | 21.4 |
| 18.24 | 13109 | 27.8 |
| 18.72 | 18966 | 47.0 |
| 18.82 | 12472 | 26.1 |
| 18.98 | 8764 | 14.5 |
| 19.38 | 11632 | 23.8 |
| 20.10 | 8776 | 15.5 |
| 20.20 | 9794 | 18.8 |
| 20.75 | 7679 | 12.5 |
| 20.94 | 6987 | 10.5 |
| 22.02 | 9107 | 18.4 |
| 25.61 | 25431 | 72.7 |
| 26.00 | 32028 | 94.1 |
| 26.65 | 21578 | 60.7 |
| 26.74 | 19565 | 54.3 |
| 27.38 | 9764 | 23.5 |
| 28.84 | 7673 | 17.4 |
| 30.24 | 7081 | 16.1 |
| 30.57 | 5568 | 11.3 |

### Example 13: Preparation and Characterization of Crystalline Glycine Cocrystal of Compound 1

Compound 1 (50 mg) and glycine (2 eq) were placed in acetone (0.8 mL) and then water (0.4 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

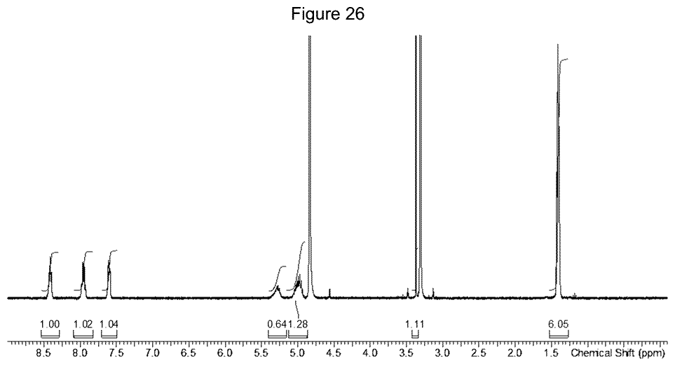

The ¹H NMR spectrum of the glycine cocrystal of Compound 1 is shown in FIG. 26.

¹H NMR (CD₃OD) δ 8.41 ppm (1H, bd), 7.95 ppm (1H, bm) 7.60 ppm (1H, bm), 5.39-4.92 ppm (2H, bm), 3.37 ppm (2H, s), 1.41 ppm (6H, bm).

The XRPD pattern of glycine cocrystal of Compound 1 is shown in FIG. 25. The peak positions, peak heights, and relative intensities of the peaks in the XRPD pattern are listed in Table 15.

TABLE 15
XRPD Peaks of glycine
cocrystal of Compound 1

| | | Relative |
|---|---|---|
| Position | Height | Intensity |
| [°2 Theta] | [counts] | [%] |
| 5.85 | 11715 | 19.3 |
| 8.62 | 20389 | 40.7 |
| 13.47 | 7901 | 12.2 |
| 14.04 | 26597 | 55.6 |
| 14.60 | 12583 | 23.0 |
| 14.79 | 19100 | 38.0 |
| 15.42 | 11329 | 20.2 |
| 17.37 | 7290 | 10.8 |
| 18.07 | 9392 | 15.6 |
| 18.59 | 7339 | 10.9 |
| 19.22 | 7040 | 10.2 |
| 20.38 | 8296 | 13.1 |
| 21.51 | 10165 | 17.5 |
| 25.67 | 14317 | 26.8 |
| 26.04 | 45909 | 100.0 |
| 26.35 | 14750 | 28.2 |
| 26.70 | 21473 | 43.7 |
| 27.12 | 9629 | 16.5 |
| 27.88 | 13843 | 26.2 |
| 28.32 | 7999 | 12.8 |
| 28.75 | 8525 | 14.0 |
| 29.40 | 7135 | 10.8 |

### Example 14: Preparation and Characterization of Crystalline D-Gluconic Acid Cocrystal of Compound 1

Compound 1 (200 mg) and D-gluconic acid (1 eq) were placed in acetone (2.7 mL) and then water (1.3 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. under vacuum overnight.

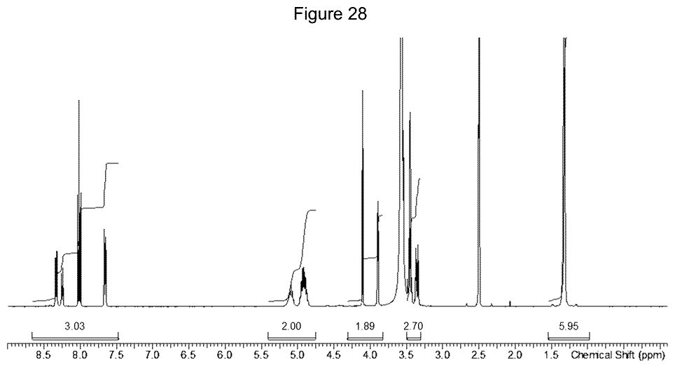

The ¹H NMR spectrum of the D-gluconic acid cocrystal of Compound 1 is shown in FIG. 28.

¹H NMR (DMSO-d₆) δ 8.33 and 8.25 ppm (1H, bdₛ), 8.01 ppm (1H, t) 7.66 ppm (1H, bd), 5.20-4.83 ppm (2H, bm), 4.11 ppm (1H, d), 3.89 ppm (1H, dd), 3.55-3.32 ppm (4H, m), 1.33 ppm (6H, bd).

The XRPD pattern of D-gluconic acid cocrystal of Compound 1 is shown in FIG. 27. The peak positions, peak heights, and relative intensities of the peaks in the XRPD pattern are listed in Table 16.

TABLE 16
XRPD Peaks of D-gluconic acid
cocrystal of Compound 1

| | | Relative |
|---|---|---|
| Position | Height | Intensity |
| [°2 Theta] | [counts] | [%] |
| 6.17 | 28578 | 44.2 |
| 7.12 | 13840 | 12.7 |
| 8.53 | 12464 | 11.6 |
| 14.14 | 34492 | 64.8 |
| 14.28 | 13254 | 18.1 |
| 14.48 | 11731 | 14.8 |
| 15.32 | 39233 | 75.6 |
| 16.38 | 11185 | 14.3 |
| 17.11 | 10374 | 12.8 |
| 17.79 | 20148 | 34.7 |
| 19.17 | 27628 | 51.5 |
| 19.32 | 35607 | 69.1 |
| 20.04 | 9247 | 11.6 |
| 20.64 | 17904 | 30.7 |
| 20.98 | 21262 | 38.6 |
| 21.32 | 31950 | 62.2 |
| 22.64 | 12736 | 21.0 |
| 22.77 | 14362 | 24.7 |
| 24.89 | 9956 | 15.8 |
| 25.59 | 48337 | 100.0 |
| 26.35 | 25290 | 49.6 |
| 26.60 | 42268 | 86.8 |
| 27.02 | 29592 | 59.3 |
| 27.65 | 21232 | 41.0 |
| 28.86 | 20771 | 40.5 |
| 29.51 | 7605 | 11.8 |
| 30.65 | 7270 | 11.5 |
| 31.89 | 6579 | 10.3 |

### Example 15: Preparation and Characterization of Crystalline Glycolic Acid Cocrystal of Compound 1

Compound 1 (400 mg) and glycolic acid (1 eq) were placed in ethyl acetate (1.25 mL). The mixture was stirred and heated up to 50° C. at 1° C./min, followed by 2 hours at 50° C. and then cooled down to 20° C. at 0.1° C./min. The mixture was then maintained under stirring at 20° C. for 3 days before filtration and drying at 40° C.

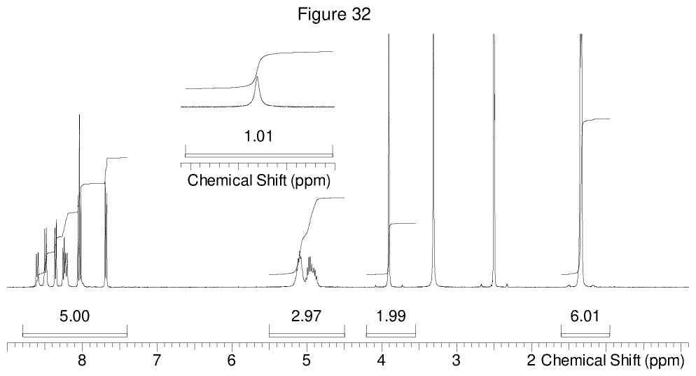

The ¹H NMR spectrum of the glycolic acid cocrystal of Compound 1 is shown in FIG. 32. ¹H NMR (DMSO-d₆) δ 12.31 ppm (1H, bs), 8.60 ppm/8.49 ppm/8.36 ppm/8.25 ppm and 8.21 ppm (3H, bdₛ), 8.04 ppm (1H, bt) 7.68 ppm (1H, bd), 5.20-4.80 ppm (2H, bm), 5.20-4.80 ppm (1H, bs), 3.91 ppm (2H, s), 1.34 ppm (6H, bd).

The XRPD pattern of glycolic acid cocrystal of Compound 1 is shown in FIG. 33. The peak positions, peak heights, and relative intensities of the peaks in the XRPD pattern are listed in Table 17.

TABLE 17
XRPD Peaks of glycolic acid
cocrystal of Compound 1

| | | Relative |
|---|---|---|
| Position | Height | Intensity |
| [°2 Theta] | [counts] | [%] |
| 6.33 | 13647 | 18.4 |
| 7.90 | 42056 | 100.0 |
| 8.41 | 14509 | 24.2 |
| 14.04 | 36299 | 90.4 |
| 15.09 | 8620 | 13.9 |
| 16.03 | 11061 | 21.3 |
| 16.25 | 16928 | 37.8 |
| 17.29 | 8069 | 13.7 |
| 17.47 | 8648 | 15.4 |
| 17.81 | 8433 | 15.1 |
| 18.21 | 9166 | 17.4 |
| 19.73 | 9397 | 18.9 |
| 20.24 | 7936 | 15.0 |
| 20.38 | 6705 | 11.7 |
| 20.68 | 10186 | 21.6 |
| 21.40 | 13388 | 30.8 |
| 22.16 | 7354 | 14.1 |
| 25.18 | 17799 | 44.0 |
| 26.00 | 16077 | 39.5 |
| 26.43 | 12508 | 29.5 |
| 26.62 | 11625 | 27.0 |
| 27.13 | 7892 | 16.9 |

### Example 16: Preparation and Characterization of Crystalline L-Malic Acid Cocrystal of Compound 1

Compound 1 (400 mg) and L-malic acid (1 eq) were placed in ethyl acetate (1 mL). The mixture was stirred and heated up to 50° C. at 1° C./min, followed by 2 hours at 50° C. and then cooled down to 20° C. at 0.1° C./min. The mixture was then maintained under stirring at 20° C. for 3 days before filtration and drying at 40° C.

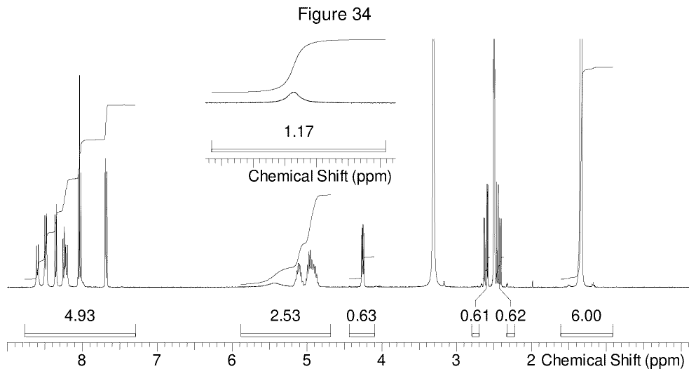

The ¹H NMR spectrum of the L-malic acid cocrystal of Compound 1 is shown in FIG. 34. ¹H NMR (DMSO-d⁶) δ 12.35 ppm (2H, bs), 8.60 ppm/8.49 ppm/8.36 ppm and 8.25 ppm 8.21 ppm (3H, bdₛ), 8.04 ppm (1H, bt) 7.69 ppm (1H, bd), 5.45 ppm (1H, bs), 5.20-4.83 ppm (2H, bm), 4.25 ppm (1H, dd), 2.61 ppm (1H, dd), 2.44 ppm (1H, dd), 1.34 ppm (6H, bd). The XRPD pattern of glycolic acid cocrystal of Compound 1 is shown in FIG. 35. The peak positions, peak heights, and relative intensities of the peaks in the XRPD pattern are listed in Table 18.

TABLE 18
XRPD Peaks of L-malic acid
cocrystal of Compound 1

| | | Relative |
|---|---|---|
| Position | Height | Intensity |
| [°2 Theta] | [counts] | [%] |
| 5.87 | 36065 | 89.7 |
| 7.25 | 11137 | 14.9 |
| 8.49 | 19580 | 43.1 |
| 13.17 | 15827 | 36.1 |
| 13.84 | 14561 | 32.6 |
| 14.13 | 23399 | 59.8 |
| 14.77 | 11609 | 24.3 |
| 15.40 | 16827 | 40.9 |
| 15.69 | 16276 | 39.1 |
| 16.51 | 9439 | 18.8 |
| 17.79 | 8905 | 17.9 |
| 18.00 | 13832 | 33.1 |
| 18.24 | 6270 | 10.1 |
| 19.22 | 7819 | 15.5 |
| 19.55 | 16193 | 41.2 |
| 20.20 | 12301 | 29.8 |
| 21.16 | 6931 | 13.8 |
| 22.41 | 10349 | 24.8 |
| 25.48 | 5307 | 10.1 |
| 25.84 | 24386 | 68.6 |
| 26.37 | 22195 | 62.1 |
| 26.52 | 34536 | 100.0 |
| 27.26 | 12885 | 33.8 |
| 27.90 | 6651 | 14.9 |
| 28.82 | 7555 | 17.9 |
| 30.27 | 5004 | 10.4 |

### Example 17: Preparation and Characterization of Crystalline Oxalic Acid Cocrystal of Compound 1

Compound 1 (1 g) and oxalic acid (1 eq) were placed in acetone (10 mL) and then water (5 mL) was added. The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C.

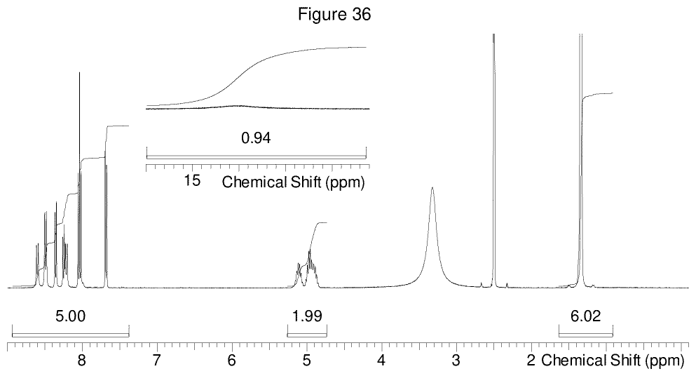

The ¹H NMR spectrum of the oxalic acid cocrystal of Compound 1 is shown in FIG. 36.

¹H NMR (DMSO-d⁶) δ 14.10 ppm (1H, vbs), 8.60 ppm/8.49 ppm/8.35 ppm/8.25 ppm and 8.21 ppm (3H, bdₛ), 8.04 ppm (1H, bt) 7.68 ppm (1H, bd), 5.20-4.80 ppm (2H, bm), 1.34 ppm (6H, bd).

The XRPD pattern of oxalic acid cocrystal of Compound 1 is shown in FIG. 37. The peak positions, peak heights, and relative intensities of the peaks in the XRPD pattern are listed in Table 19.

TABLE 19
XRPD Peaks of oxalic
cocrystal of Compound 1

| | | Relative |
|---|---|---|
| Position | Height | Intensity |
| [°2 Theta] | [counts] | [%] |
| 5.81 | 53444 | 100.0 |
| 8.53 | 12182 | 11.6 |
| 11.65 | 11382 | 12.9 |
| 13.67 | 21864 | 36.9 |
| 14.26 | 10492 | 11.7 |
| 14.90 | 21643 | 36.8 |
| 18.08 | 11903 | 16.1 |
| 26.18 | 16818 | 29.9 |
| 26.54 | 10139 | 15.1 |
| 26.82 | 21409 | 40.1 |
| 27.18 | 11042 | 17.4 |
| 27.54 | 8287 | 11.4 |

### Example 18: Preparation and Characterization of Crystalline Benzoic Acid Cocrystal of Compound 1

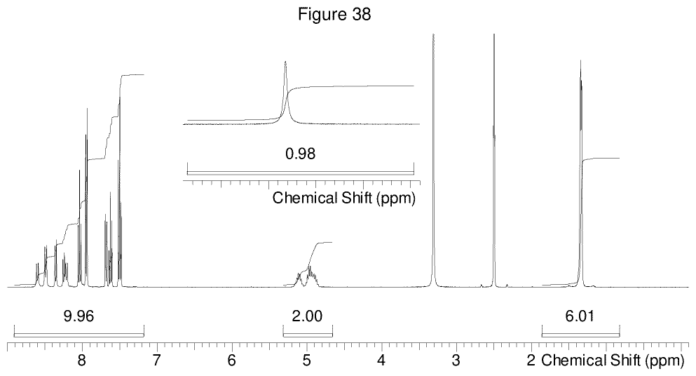

Compound 1 (2 g) and benzoic acid (1 eq) were placed in ethyl acetate (5 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C. The cocrystal was analyzed by XRPD, ¹H NMR. The ¹H NMR spectrum of the benzoic acid cocrystal of Compound 1 is shown in FIG. 38. ¹H NMR (DMSO-d⁶) δ12.91 ppm (1H, bs), 8.60 ppm/8.49 ppm/8.35 ppm/8.25 ppm and 8.21 ppm (3H, bdₛ), 8.04 ppm (1H, bt), 7.95 ppm (2H, m), 7.68 ppm (1H, bd), 7.62 ppm (1H, m), 7.50 ppm (2H, m), 5.20-4.80 ppm (2H, bm), 1.34 ppm (6H, bd).

The XRPD pattern of benzoic acid cocrystal of Compound 1 is shown in FIG. 39. The peak positions, peak heights, and relative intensities of the peaks in the XRPD pattern are listed in Table 20.

TABLE 20
XRPD Peaks of benzoic acid
cocrystal of Compound 1

| | | Relative |
|---|---|---|
| Position | Height | Intensity |
| [°2 Theta] | [counts] | [%] |
| 6.00 | 47587 | 100.0 |
| 8.50 | 37371 | 78.0 |
| 9.80 | 33056 | 69.6 |
| 11.53 | 10939 | 13.9 |
| 13.92 | 16640 | 30.3 |
| 14.34 | 10050 | 13.7 |
| 15.14 | 11788 | 18.5 |
| 16.31 | 16268 | 30.7 |
| 17.05 | 17913 | 35.0 |
| 17.37 | 20276 | 41.2 |
| 17.70 | 11341 | 18.7 |
| 18.05 | 36598 | 82.7 |
| 19.04 | 17240 | 34.3 |
| 19.68 | 15058 | 29.0 |
| 19.99 | 33169 | 75.0 |
| 20.60 | 15902 | 31.5 |
| 20.89 | 20674 | 44.0 |
| 21.51 | 25965 | 57.5 |
| 22.03 | 12219 | 22.9 |
| 22.32 | 16881 | 34.8 |
| 22.58 | 22050 | 47.9 |
| 22.85 | 7828 | 12.1 |
| 23.64 | 27001 | 60.9 |
| 24.93 | 16253 | 34.1 |
| 26.13 | 14269 | 29.5 |
| 26.36 | 8644 | 15.4 |
| 27.06 | 24593 | 56.0 |
| 28.19 | 6624 | 10.8 |
| 28.65 | 6604 | 10.9 |
| 29.70 | 10692 | 21.2 |
| 30.96 | 7030 | 12.7 |
| 31.57 | 7177 | 13.2 |

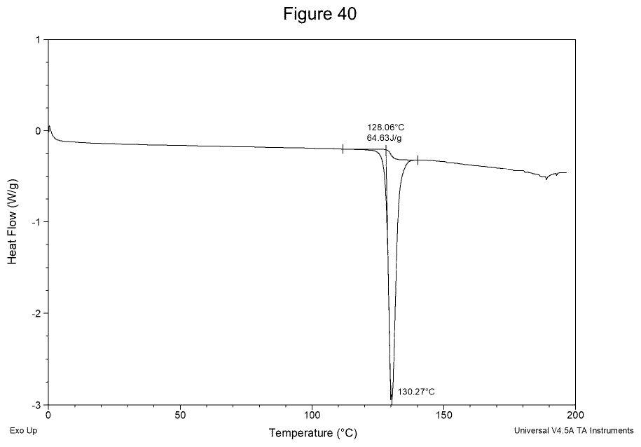

The DSC thermogram of the benzoic acid cocrystal of Compound 1 depicted in FIG. 40 comprises an endothermic peak having an onset temperature of 128.06° C. and a peak temperature of 130.27° C.

### Example 19: Preparation and characterization of crystalline fumaric acid cocrystal of Compound 1

Compound 1 (400 mg) and fumaric acid (1 eq) were placed in methanol (1.5 mL). The mixture was stirred and heated up to 50° C. at 1° C./min, followed by 2 hours at 50° C. and then cooled down to 20° C. at 0.1° C./min. The mixture was then maintained under stirring at 20° C. for 3 days before filtration and drying at 40° C.

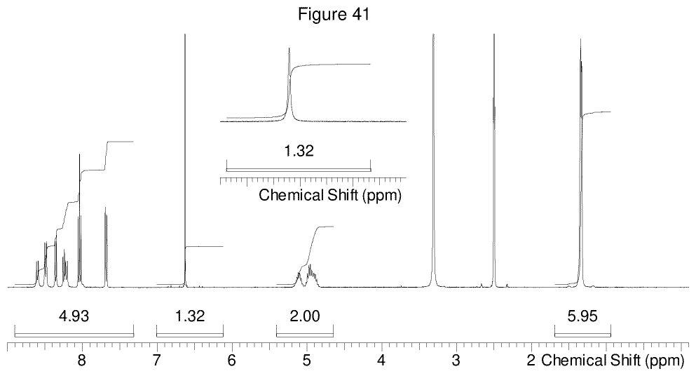

The ¹H NMR spectrum of the fumaric acid cocrystal of Compound 1 is shown in FIG. 41.

¹H NMR (DMSO-d⁶) δ 13.10 ppm (2H, bs), 8.60 ppm/8.49 ppm/8.35 ppm/8.25 ppm and 8.21 ppm (3H, bdₛ), 8.04 ppm (1H, bt) 7.68 ppm (1H, bd), 6.63 ppm (2H, s), 5.20-4.80 ppm (2H, bm), 1.34 ppm (6H, d).

The XRPD pattern of fumaric acid cocrystal of Compound 1 is shown in FIG. 42. The peak positions, peak heights, and relative intensities of the peaks in the XRPD pattern are listed in Table 21.

TABLE 21
XRPD Peaks of fumaric acid
cocrystal of Compound 1

| | | Relative |
|---|---|---|
| Position | Height | Intensity |
| [°2 Theta] | [counts] | [%] |
| 5.59 | 54346 | 100.0 |
| 9.12 | 14862 | 20.3 |
| 9.45 | 21340 | 34.4 |
| 11.19 | 10260 | 11.7 |
| 11.83 | 10637 | 13.0 |
| 13.63 | 16135 | 25.5 |
| 13.76 | 9750 | 12.0 |
| 14.08 | 13995 | 21.2 |
| 14.25 | 26094 | 47.0 |
| 15.64 | 9295 | 11.8 |
| 18.48 | 8762 | 11.6 |
| 18.98 | 8698 | 11.4 |
| 19.46 | 13570 | 22.1 |
| 20.16 | 10210 | 15.3 |
| 20.45 | 9269 | 13.4 |
| 21.60 | 13078 | 22.0 |
| 22.46 | 10586 | 17.1 |
| 25.91 | 26640 | 50.8 |
| 26.34 | 16429 | 29.8 |
| 26.67 | 10658 | 17.6 |
| 27.51 | 14152 | 25.2 |

### Example 20: Preparation and characterization of crystalline gentisic acid cocrystal of Compound 1

Compound 1 (400 mg) and gentisic acid (1 eq) were placed in ethyl acetate (1.5 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C.

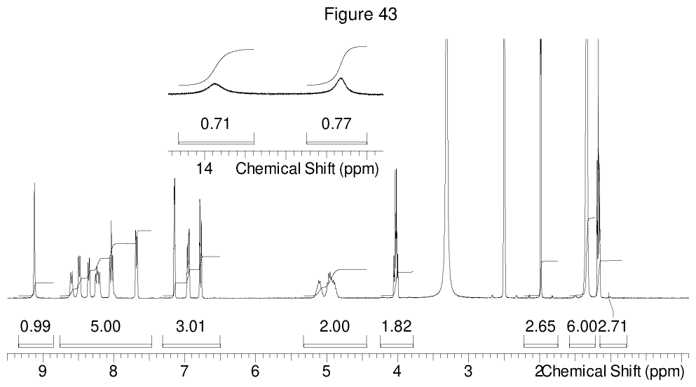

The ¹H NMR spectrum of the gentisic acid cocrystal of Compound 1 is shown in FIG. 43. ¹H NMR (DMSO-d⁶) δ 13.75 ppm (1H, bs), 10.65 ppm (1H, bs), 9.12 ppm (1H, s), 8.60 ppm/8.49 ppm/8.35 ppm/8.25 ppm and 8.21 ppm (3H, bdₛ), 8.04 ppm (1H, t), 7.68 ppm (1H, bd), 7.15 ppm (1H, bm), 6.95 ppm (1H, bm), 6.78 ppm (1H, bm), 5.20-4.82 ppm (2H, bm), 1.34 ppm (6H, bd).

The XRPD pattern of gentisic acid cocrystal of Compound 1 is shown in FIG. 44. The peak positions, peak heights, and relative intensities of the peaks in the XRPD pattern are listed in Table 22.

TABLE 22
XRPD Peaks of gentisic acid
cocrystal of Compound 1

| | | Relative |
|---|---|---|
| Position | Height | Intensity |
| [°2 Theta] | [counts] | [%] |
| 6.35 | 17697 | 40.8 |
| 6.46 | 29122 | 86.6 |
| 6.52 | 26219 | 70.1 |
| 11.05 | 8582 | 14.7 |
| 12.63 | 22392 | 71.3 |
| 12.72 | 15736 | 45.1 |
| 12.96 | 8224 | 15.4 |
| 13.44 | 14646 | 41.2 |
| 13.84 | 29018 | 98.2 |
| 15.01 | 7766 | 15.7 |
| 16.57 | 8942 | 21.5 |
| 16.72 | 7439 | 15.9 |
| 16.86 | 14217 | 42.8 |
| 17.34 | 19106 | 62.8 |
| 17.95 | 6294 | 12.6 |
| 18.83 | 5694 | 11.1 |
| 19.15 | 8625 | 23.1 |
| 20.37 | 6273 | 14.6 |
| 20.88 | 5471 | 11.6 |
| 21.67 | 6182 | 15.1 |
| 23.05 | 6414 | 16.6 |
| 23.19 | 6484 | 16.0 |
| 23.62 | 4992 | 11.1 |
| 24.81 | 11844 | 38.3 |
| 25.44 | 6719 | 18.0 |
| 26.08 | 27486 | 100.0 |
| 26.50 | 19938 | 70.4 |
| 27.10 | 11264 | 36.3 |
| 27.30 | 7965 | 23.6 |

### Example 21: Preparation and Characterization of Crystalline Glutaric Acid Cocrystal of Compound 1

Compound 1 (400 mg) and glutaric acid (1 eq) were placed in ethyl acetate (0.5 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration and drying at 40° C.

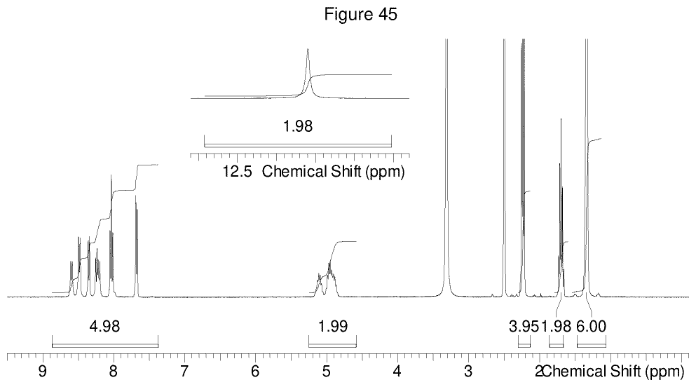

The ¹H NMR spectrum of the glutaric acid cocrystal of Compound 1 is shown in FIG. 45.

¹H NMR (DMSO-d⁶) δ 12.05 ppm (1H, s), 8.60 ppm/8.48 ppm/8.35 ppm/8.25 and 8.21 ppm (3H, bdₛ), 8.03 ppm (1H, t), 7.68 ppm (1H, bd), 5.20-4.82 ppm (2H, bm), 2.24 ppm (4H, bt), 1.70 ppm (2H, qi), 1.34 ppm (6H, bd).

The XRPD pattern of glutaric acid cocrystal of Compound 1 is shown in FIG. 46. The peak positions, peak heights, and relative intensities of the peaks in the XRPD pattern are listed in Table 23.

TABLE 23
XRPD Peaks of glutaric
acid cocrystal of Compound 1

| Position | Height | Relative |
|---|---|---|
| [°2 Theta] | [counts] | Intensity [%] |
| 5.76 | 8992 | 14.0 |
| 6.72 | 12616 | 59.3 |
| 7.19 | 16147 | 100.0 |
| 8.37 | 6964 | 11.5 |
| 8.58 | 8602 | 29.7 |
| 10.15 | 7229 | 22.0 |
| 12.00 | 7735 | 34.9 |
| 12.65 | 8673 | 46.2 |
| 13.76 | 6022 | 20.5 |
| 14.00 | 6658 | 27.0 |
| 14.30 | 5761 | 17.3 |
| 15.23 | 8216 | 44.7 |
| 15.65 | 6144 | 24.6 |
| 16.59 | 6491 | 29.7 |
| 16.81 | 6170 | 27.2 |
| 17.50 | 4555 | 11.5 |
| 18.39 | 4584 | 13.8 |
| 18.78 | 4326 | 12.5 |
| 19.13 | 5355 | 24.0 |
| 19.60 | 4801 | 18.6 |
| 20.49 | 6150 | 34.9 |
| 21.10 | 5200 | 26.7 |
| 21.30 | 6668 | 42.6 |
| 21.72 | 6564 | 42.4 |
| 22.11 | 4056 | 16.7 |
| 22.45 | 4405 | 21.4 |
| 24.41 | 4068 | 18.7 |
| 25.45 | 7410 | 55.2 |
| 25.84 | 4476 | 25.0 |
| 26.34 | 5394 | 35.4 |
| 27.00 | 4178 | 23.5 |
| 27.25 | 3062 | 11.7 |
| 27.73 | 2811 | 10.9 |
| 29.14 | 2634 | 11.3 |

### Example 22: Preparation and Characterization of Crystalline Ortho Phosphoric Acid Salt of Compound 1

Compound 1 (300 mg) and ortho phosphoric acid (1 eq.) were placed in ethyl acetate (9 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

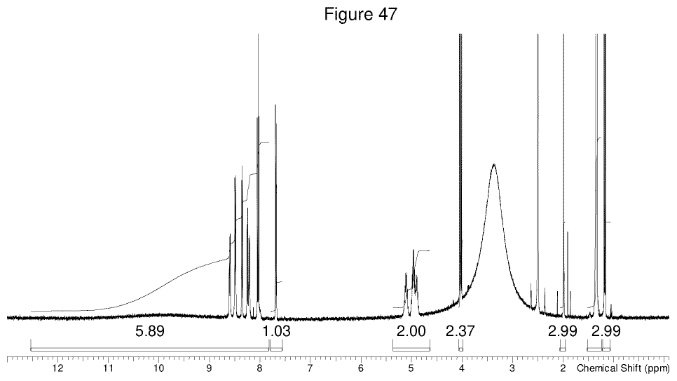

The ¹H NMR spectrum of the ortho phosphoric acid salt of Compound 1 is shown in FIG. 47.

¹H NMR (DMSO-d⁶) δ 9.90 ppm (3H, bs), 8.60 ppm/8.49 ppm/8.35 ppm/8.25 and 8.21 ppm (3H, bdₛ), 8.04 ppm (1H, t), 7.89 ppm (1H, bs), 7.68 ppm (1H, bd), 5.20-4.82 ppm (2H, bmₛ), 1.34 ppm (6H, bd)

The XRPD pattern of ortho phosphoric acid salt of Compound 1 is shown in FIG. 48. The peak positions, peak heights, and relative intensities of the peaks in the XRPD pattern are listed in Table 24.

TABLE 24
XRPD Peaks of ortho phosphoric
acid salt of Compound 1

| Pos. | Intensity | Rel. Int. |
|---|---|---|
| [°2θ] | [Counts] | [%] |
| 3.65 | 16250 | 19.2 |
| 5.47 | 18911 | 28.5 |
| 7.04 | 12258 | 16.9 |
| 11.01 | 10219 | 15.9 |
| 14.27 | 14769 | 27.2 |
| 14.69 | 10936 | 20.1 |
| 14.91 | 15292 | 27.3 |
| 18.35 | 41948 | 100.0 |
| 19.72 | 15528 | 31.8 |
| 20.34 | 10107 | 20.0 |
| 21.29 | 16701 | 36.4 |
| 26.53 | 8390 | 13.2 |
| 27.09 | 9700 | 17.5 |
| 27.40 | 9291 | 11.5 |
| 27.67 | 8465 | 14.0 |

### Example 23: Preparation and Characterization of Crystalline 4-Hydroxybenzoic Acid Cocrystal of Compound 1

Compound 1 (300 mg) and 4-hydroxybenzoic acid (1 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

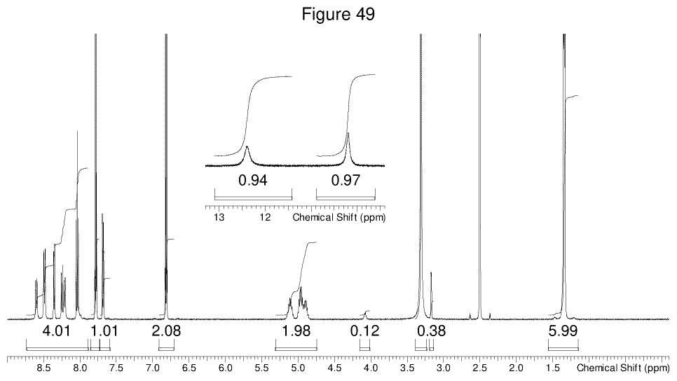

The ¹H NMR spectrum of the 4-hydroxybenzoic acid cocrystal of Compound 1 is shown in FIG. 49.

¹H NMR (DMSO-d⁶) δ 12.39 ppm (1H, bs), 10.20 ppm (1H, bs), 8.60 ppm/8.49 ppm/8.35 ppm/8.25 and 8.21 ppm (3H, bdₛ), 8.04 ppm (1H, t), 7.78 ppm (2H, m), 7.68 ppm (1H, bd), 6.81 ppm (2H, m), 5.20-4.82 ppm (2H, bmₛ), 1.34 ppm (6H, bd).

The XRPD pattern of 4-hydroxybenzoic acid cocrystal of Compound 1 is shown in FIG. 50. The peak positions, peak heights, and relative intensities of the peaks in the XRPD pattern are listed in Table 25.

TABLE 25
XRPD Peaks of 4-hydroxybenzoic
acid cocrystal of Compound 1

| Pos. | Intensity | Rel. Int. |
|---|---|---|
| [°2θ] | [Counts] | [%] |
| 5.90 | 10515 | 19.2 |
| 6.67 | 25400 | 100.0 |
| 7.63 | 8476 | 13.1 |
| 7.94 | 7857 | 11.3 |
| 14.18 | 6591 | 14.2 |
| 14.64 | 14113 | 58.9 |
| 15.81 | 8325 | 26.6 |
| 17.52 | 10365 | 40.3 |
| 18.91 | 7512 | 26.4 |
| 20.36 | 5428 | 13.9 |
| 20.59 | 14718 | 69.1 |
| 22.46 | 4890 | 12.9 |
| 24.42 | 6264 | 23.8 |
| 26.40 | 9246 | 39.1 |
| 26.88 | 8643 | 32.6 |
| 27.71 | 4956 | 15.6 |
| 29.07 | 4358 | 14.7 |

### Example 24: Preparation and Characterization of Crystalline Alpha-Ketoglutaric Acid Cocrystal of Compound 1

Compound 1 (300 mg) and alpha-ketoglutaric acid (1 eq.) were placed in ethyl acetate (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

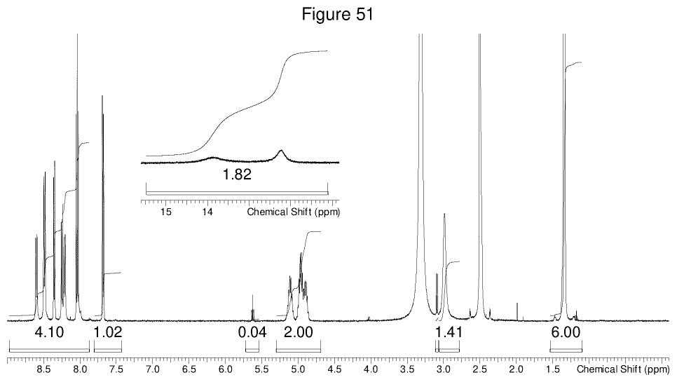

The ¹H NMR spectrum of the alpha ketoglutaric acid cocrystal of Compound 1 is shown in FIG. 51.

¹H NMR (DMSO-d⁶) δ 13.88 ppm (1H, bs), 12.22 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 2.99 ppm (4H, bs), 1.34 ppm (6H, bd)

The XRPD pattern of alpha-ketoglutaric acid cocrystal of Compound 1 is shown in FIG. 52. The peak positions, peak heights, and relative intensities of the peaks in the XRPD pattern are listed in Table 26.

TABLE 26
XRPD Peaks of alpha-ketoglutaric
acid cocrystal of Compound 1

| Pos. | Intensity | Rel. Int. |
|---|---|---|
| [°2θ] | [Counts] | [%] |
| 5.89 | 57745 | 100.0 |
| 7.39 | 23206 | 36.9 |
| 7.95 | 17001 | 23.4 |
| 14.30 | 8866 | 12.1 |
| 14.95 | 11414 | 15.8 |
| 15.12 | 10362 | 13.6 |
| 16.17 | 9249 | 12.0 |
| 17.30 | 20482 | 40.3 |
| 17.61 | 13825 | 25.1 |
| 19.00 | 12575 | 23.9 |
| 19.74 | 10442 | 15.7 |
| 21.01 | 14593 | 27.7 |
| 23.11 | 7806 | 11.7 |
| 26.22 | 14050 | 30.4 |
| 26.89 | 20659 | 48.5 |

### Example 25: Preparation and Characterization of Crystalline Malonic Acid Cocrystal of Compound 1

Compound 1 (300 mg) and malonic acid (1 eq.) were placed in ethyl acetate (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

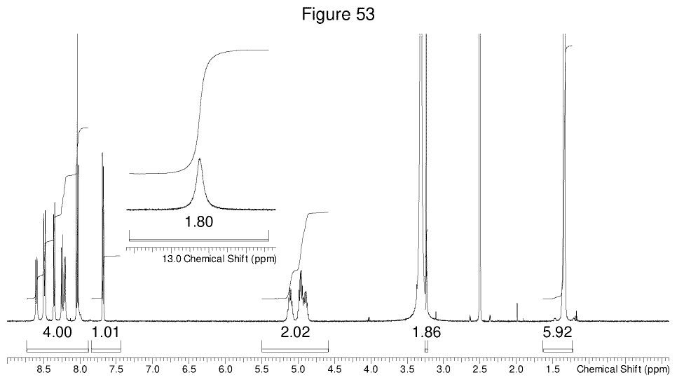

The ¹H NMR spectrum of the malonic acid cocrystal of Compound 1 is shown in FIG. 53.

¹H NMR (DMSO-d⁶) δ 12.62 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 3.24 ppm (2H, bs), 1.34 ppm (6H, bd)

The XRPD pattern of malonic acid cocrystal of Compound 1 is shown in FIG. 54. The peak positions, peak heights, and relative intensities of the peaks in the XRPD pattern are listed in Table 27.

TABLE 27
XRPD Peaks of malonic acid
cocrystal of Compound 1

| Pos. | Intensity | Rel. Int. |
|---|---|---|
| [°2θ] | [Counts] | [%] |
| 7.97 | 75334 | 100.0 |
| 13.74 | 23994 | 29.8 |
| 14.74 | 16555 | 19.6 |
| 16.07 | 20295 | 24.5 |
| 16.26 | 12561 | 13.2 |
| 18.95 | 9456 | 10.0 |
| 19.34 | 24841 | 28.2 |
| 19.47 | 16246 | 18.8 |
| 21.01 | 10315 | 11.2 |
| 21.85 | 9256 | 10.8 |
| 23.70 | 9396 | 10.8 |
| 25.92 | 16085 | 20.6 |
| 27.15 | 14882 | 18.0 |

### Example 26: Preparation and Characterization of Crystalline Salicylic Acid Cocrystal of Compound 1

Compound 1 (300 mg) and salicylic acid (1 eq.) were placed in ethyl acetate (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

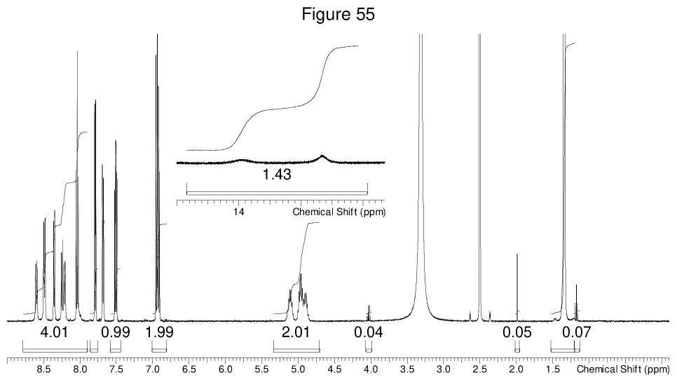

The ¹H NMR spectrum of the salicylic acid cocrystal of Compound 1 is shown in FIG. 55.

¹H NMR (DMSO-d⁶) δ 13.90 ppm (1H, bs), 11.32 ppm (1H, bs), 8.60 ppm/8.49 ppm/8.35 ppm/8.25 and 8.21 ppm (3H, bdₛ), 8.04 ppm (1H, t), 7.79 ppm (1H, dd), 7.68 ppm (1H, bd), 7.51 ppm (1H, ddd), 6.97-6.89 ppm (2H, m), 5.20-4.82 ppm (2H, bmₛ), 1.34 ppm (6H, bd)

The XRPD pattern of salicylic acid cocrystal of Compound 1 is shown in FIG. 56. The peak positions, peak heights, and relative intensities of the peaks in the XRPD pattern are listed in Table 28.

TABLE 28
XRPD Peaks of salicylic acid
cocrystal of Compound 1

| Pos. | Intensity | Rel. Int. |
|---|---|---|
| [°2θ] | [Counts] | [%] |
| 6.19 | 21066 | 70.0 |
| 6.77 | 22952 | 80.7 |
| 7.29 | 22530 | 80.4 |
| 13.89 | 18419 | 78.2 |
| 14.69 | 14865 | 60.8 |
| 16.36 | 21083 | 100.0 |
| 17.35 | 19627 | 77.6 |
| 18.24 | 17338 | 82.0 |
| 18.37 | 12653 | 50.4 |
| 19.02 | 11802 | 53.3 |
| 20.76 | 14467 | 68.1 |
| 21.21 | 19177 | 91.6 |
| 25.74 | 14990 | 68.8 |
| 26.32 | 16855 | 51.8 |
| 26.89 | 12866 | 54.3 |

### Example 27: Preparation and Characterization of Crystalline L-Tartaric Acid Cocrystal of Compound 1

Compound 1 (150 mg) and L-tartaric acid (1 eq.) were placed in ethyl acetate (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

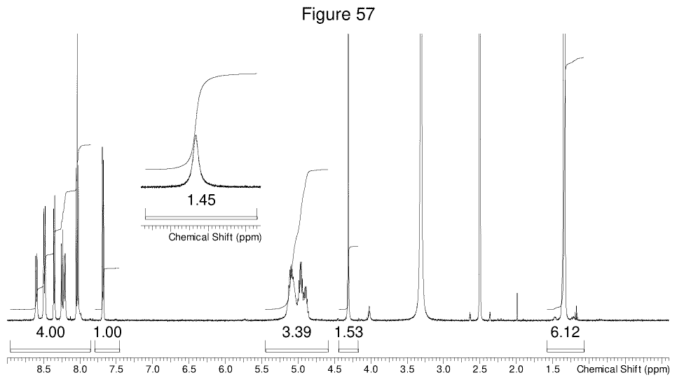

The ¹H NMR spectrum of the L-tartaric acid cocrystal of Compound 1 is shown in FIG. 57.

¹H NMR (DMSO-d⁶) δ 12.66 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 4.31 ppm (2H, s), 1.34 ppm (6H, bd)

The XRPD pattern of L-tartaric acid cocrystal of Compound 1 is shown in FIG. 58. The peak positions, peak heights, and relative intensities of the peaks in the XRPD pattern are listed in Table 29.

TABLE 29
XRPD Peaks of L-tartaric acid
cocrystal of Compound 1

| Pos. | Intensity | Rel. Int. |
|---|---|---|
| [°2θ] | [Counts] | [%] |
| 5.74 | 17794 | 50.0 |
| 10.16 | 11461 | 30.5 |
| 11.84 | 13992 | 45.6 |
| 12.95 | 8958 | 23.8 |
| 13.62 | 17502 | 62.8 |
| 14.31 | 11106 | 36.7 |
| 17.98 | 22388 | 100.0 |
| 18.95 | 8182 | 22.5 |
| 19.07 | 9622 | 31.5 |
| 20.41 | 8615 | 29.2 |
| 20.67 | 9495 | 35.7 |
| 20.89 | 8995 | 32.2 |
| 21.90 | 9709 | 37.6 |
| 25.02 | 6998 | 24.8 |
| 29.10 | 9276 | 40.8 |

### Example 28: Preparation and Characterization of Crystalline Urea Cocrystal of Compound 1

Compound 1 (300 mg) and urea (1 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

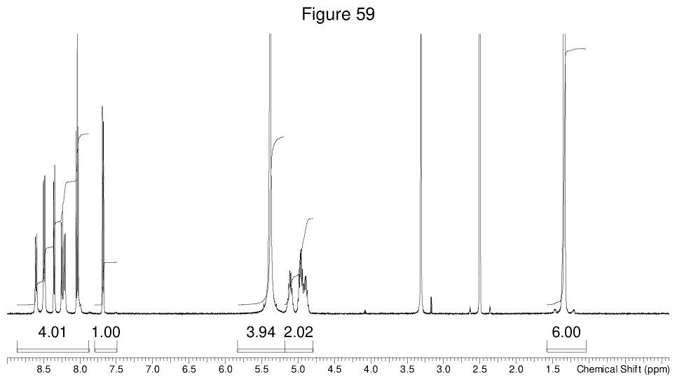

The ¹H NMR spectrum of the urea cocrystal of Compound 1 is shown in FIG. 59.

¹H NMR (DMSO-d⁶) δ 8.60 ppm/8.49 ppm/8.35 ppm/8.25 and 8.21 ppm (3H, bdₛ), 8.04 ppm (1H, t), 7.68 ppm (1H, bd), 5.39 ppm (4H, bs), 5.20-4.82 ppm (2H, bmₛ), 1.34 ppm (6H, bd)

The XRPD pattern of urea cocrystal of Compound 1 is shown in FIG. 60. The peak positions, peak heights, and relative intensities of the peaks in the XRPD pattern are listed in Table 30.

TABLE 30
XRPD Peaks of urea
cocrystal of Compound 1

| Pos. | Intensity | Rel. Int. |
|---|---|---|
| [°2θ] | [Counts] | [%] |
| 8.19 | 11721 | 16.6 |
| 8.79 | 20114 | 36.7 |
| 13.76 | 44100 | 100.0 |
| 14.93 | 12351 | 22.4 |
| 15.18 | 9630 | 16.1 |
| 16.34 | 15175 | 30.8 |
| 17.34 | 9718 | 17.5 |
| 18.76 | 9920 | 17.6 |
| 19.54 | 12744 | 23.7 |
| 20.15 | 7399 | 11.6 |
| 21.33 | 8237 | 13.9 |
| 22.50 | 12110 | 24.5 |
| 24.87 | 11737 | 23.3 |
| 25.62 | 10575 | 20.0 |
| 26.43 | 17211 | 37.8 |
| 27.14 | 10095 | 20.4 |
| 29.14 | 6127 | 11.4 |

### Example 29: Preparation and Characterization of Crystalline Pyroglutamic Acid Cocrystal of Compound 1

Compound 1 (300 mg) and pyroglutamic acid (1 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before filtration.

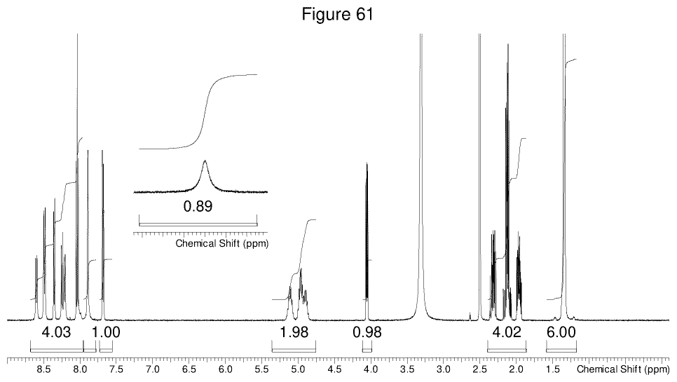

The ¹H NMR spectrum of the pyroglutamic acid cocrystal of Compound 1 is shown in FIG. 61.

¹H NMR (DMSO-d⁶) δ 12.75 ppm (1H, bs), 8.60 ppm/8.49 ppm/8.35 ppm/8.25 and 8.21 ppm (3H, bdₛ), 8.04 ppm (1H, t), 7.89 ppm (1H, bs), 7.68 ppm (1H, bd), 5.20-4.82 ppm (2H, bmₛ), 4.05 ppm (1H, ddd), 2.40-1.90 ppm (4H, ms), 1.34 ppm (6H, bd) The XRPD pattern of pyroglutamic acid cocrystal of Compound 1 is shown in FIG. 62.

The peak positions, peak heights, and relative intensities of the peaks in the XRPD pattern are listed in Table 31.

TABLE 31
XRPD Peaks of pyroglutamic
acid cocrystal of Compound 1

| Pos. | Intensity | Rel. Int. |
|---|---|---|
| [°2θ] | [Counts] | [%] |
| 6.20 | 72049 | 100.0 |
| 7.72 | 30184 | 39.0 |
| 12.57 | 24126 | 34.6 |
| 13.87 | 40634 | 64.5 |
| 14.19 | 26175 | 38.0 |
| 17.27 | 19107 | 29.4 |
| 17.44 | 17832 | 27.6 |
| 18.20 | 20995 | 34.3 |
| 20.78 | 19413 | 31.9 |
| 20.89 | 18827 | 29.8 |
| 23.20 | 13680 | 23.4 |
| 25.30 | 52099 | 96.6 |
| 25.89 | 20299 | 37.1 |
| 26.24 | 31931 | 60.5 |
| 27.26 | 32805 | 57.6 |
| 27.77 | 12611 | 21.4 |

### Example 30: Preparation and Characterization of Crystalline Caproic Acid Cocrystal of Compound 1

Compound 1 (300 mg) and caproic acid (2 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

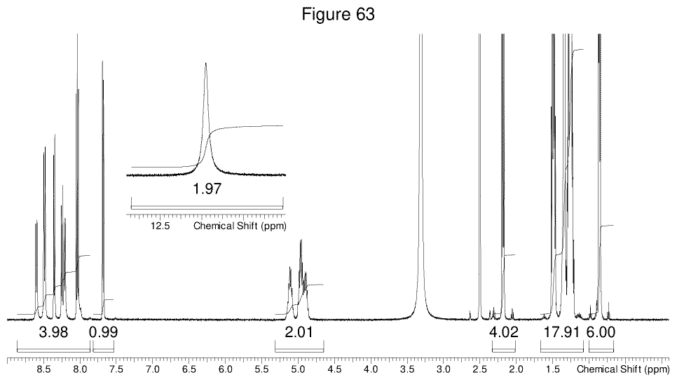

The ¹H NMR spectrum of the caproic acid cocrystal of Compound 1 is shown in FIG. 63.

¹H NMR (DMSO-d⁶) δ 11.95 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 2.18 ppm (1H, t), 1.49 ppm (2H, qi), 1.40-1.20 ppm (4H, ms), 1.34 ppm (6H, bd), 0.86 ppm (3H, t)

The XRPD pattern of caproic acid cocrystal of Compound 1 is shown in FIG. 64. The peak positions, peak heights, and relative intensities of the peaks in the XRPD pattern are listed in Table 32.

TABLE 32
XRPD Peaks of caproic acid
cocrystal of Compound 1

| Pos. | Intensity | Rel. Int. |
|---|---|---|
| [°2θ] | [Counts] | [%] |
| 8.36 | 24903 | 24.0 |
| 8.55 | 60645 | 58.5 |
| 9.80 | 40236 | 39.6 |
| 12.17 | 15414 | 13.3 |
| 12.30 | 29240 | 29.7 |
| 14.25 | 49839 | 55.1 |
| 14.39 | 13944 | 12.8 |
| 17.17 | 16605 | 16.5 |
| 18.96 | 26477 | 30.5 |
| 19.33 | 25978 | 28.0 |
| 19.60 | 31370 | 32.8 |
| 20.64 | 37725 | 46.9 |
| 20.98 | 37133 | 43.6 |
| 21.28 | 35599 | 40.7 |
| 23.10 | 78011 | 100.0 |
| 23.27 | 12739 | 11.6 |
| 23.81 | 12813 | 13.9 |
| 24.45 | 34643 | 37.1 |
| 25.80 | 22515 | 26.8 |
| 26.32 | 13664 | 14.3 |

### Example 31: Preparation and Characterization of Crystalline Glycerol Cocrystal of Compound

Compound 1 (300 mg) and glycerol (1 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

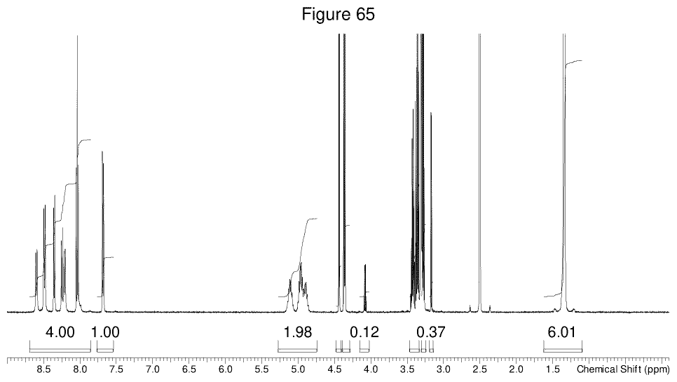

The ¹H NMR spectrum of the glycerol cocrystal of Compound 1 is shown in FIG. 65.

¹H NMR (DMSO-d⁶) δ 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 4.44 ppm (1H, d), 4.36 ppm (2H, t), 3.42 ppm (1H, qi), 3.36 ppm (2H, td), 3.28 ppm (2H, td), 1.34 ppm (6H, bd) The XRPD pattern of glycerol cocrystal of Compound 1 is shown in FIG. 66. The peak positions, peak heights, and relative intensities of the peaks in the XRPD pattern are listed in Table 33.

TABLE 33
XRPD Peaks of glycerol
cocrystal of Compound 1

| Pos. | Intensity | Rel. Int. |
|---|---|---|
| [°2θ] | [Counts] | [%] |
| 5.91 | 12892 | 38.0 |
| 8.41 | 12436 | 37.3 |
| 9.93 | 8299 | 19.3 |
| 11.85 | 15449 | 55.9 |
| 12.95 | 9698 | 29.5 |
| 13.21 | 8378 | 19.3 |
| 14.32 | 12884 | 40.0 |
| 16.35 | 8375 | 22.3 |
| 17.99 | 22359 | 100.0 |
| 18.55 | 12558 | 45.4 |
| 18.96 | 10856 | 36.0 |
| 19.99 | 8139 | 19.6 |
| 20.56 | 7710 | 18.9 |
| 20.89 | 11837 | 40.5 |
| 21.92 | 10220 | 35.1 |
| 22.41 | 7568 | 19.6 |

### Example 32: Preparation and Characterization of Crystalline L-Lysine Cocrystal of Compound 1

Compound 1 (300 mg) and L-lysine (2 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

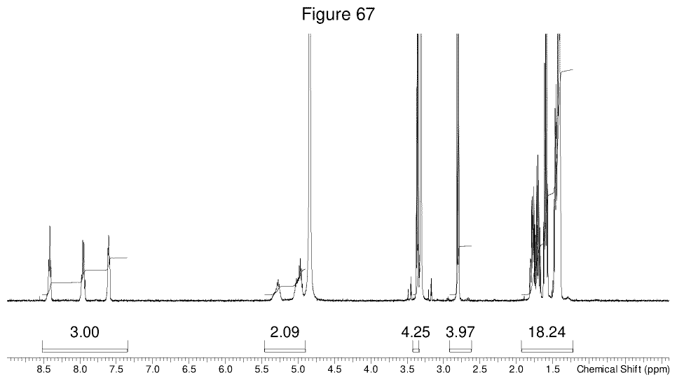

The ¹H NMR spectrum of the L-lysine cocrystal of Compound 1 is shown in FIG. 67.

¹H NMR (DMSO-d⁶) δ 8.41 ppm/7.95 ppm/7.61 ppm (3H, ms), 5.41-4.92 ppm (2H, bmₛ), 3.36 ppm (1H, t), 2.80 ppm (2H, t), 1.83-1.65 ppm (2H, m), 1.60 ppm (2H, qi), 1.50-1.36 ppm (2H, m), 1.41 ppm (6H, bm)

The XRPD pattern of L-lysine cocrystal of Compound 1 is shown in FIG. 68. The peak positions, peak heights, and relative intensities of the peaks in the XRPD pattern are listed in Table 34.

TABLE 34
XRPD Peaks of L-lysine
cocrystal of Compound 1

| Pos. | Intensity | Rel. Int. |
|---|---|---|
| [°2θ] | [Counts] | [%] |
| 5.00 | 25460 | 100.0 |
| 6.68 | 18905 | 71.1 |
| 12.61 | 18376 | 83.5 |
| 13.27 | 11240 | 40.5 |
| 13.69 | 14261 | 55.8 |
| 14.28 | 8065 | 20.7 |
| 14.87 | 9492 | 29.4 |
| 17.21 | 7666 | 20.5 |
| 17.55 | 14154 | 51.9 |
| 17.79 | 11299 | 34.6 |
| 18.50 | 16657 | 70.0 |
| 19.26 | 8684 | 20.3 |
| 19.58 | 10186 | 31.0 |
| 20.51 | 9334 | 27.8 |
| 21.03 | 12184 | 45.4 |
| 21.45 | 10466 | 36.8 |
| 22.58 | 13948 | 56.6 |
| 23.45 | 16663 | 69.4 |
| 24.44 | 12336 | 50.2 |
| 25.24 | 9952 | 38.9 |

### Example 33: Preparation and Characterization of Crystalline S-Proline Cocrystal of Compound 1

Compound 1 (300 mg) and S-proline (1 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

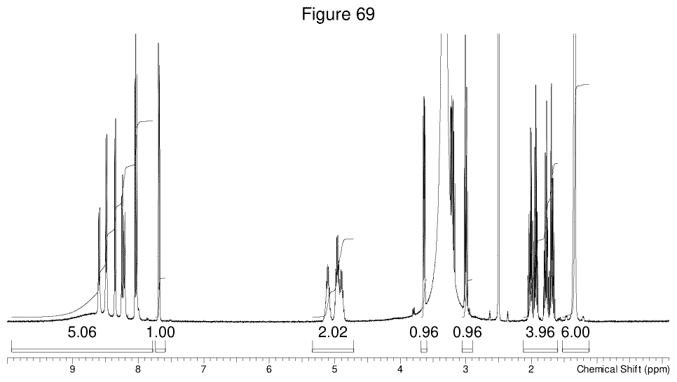

The ¹H NMR spectrum of the S-proline cocrystal of Compound 1 is shown in FIG. 69.

¹H NMR (DMSO-d⁶) δ 8.60 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 3.64 ppm (1H, dd), 3.20 ppm (1H, m), 3.00 ppm (1H, td), 1.97 ppm (2H, m), 1.73 ppm (2H, m), 1.34 ppm (6H, bd)

The XRPD pattern of S-proline cocrystal of Compound 1 is shown in FIG. 70. The peak positions, peak heights, and relative intensities of the peaks in the XRPD pattern are listed in Table 35.

TABLE 35
XRPD Peaks of S-proline
cocrystal of Compound 1

| Pos. | Intensity | Rel. Int. |
|---|---|---|
| [°2θ] | [Counts] | [%] |
| 5.91 | 19300 | 100.0 |
| 7.19 | 15199 | 67.6 |
| 14.02 | 10906 | 35.5 |
| 14.58 | 7370 | 12.7 |
| 16.22 | 8137 | 19.5 |
| 17.43 | 9407 | 19.0 |
| 18.03 | 8907 | 12.3 |
| 19.76 | 7926 | 26.5 |
| 25.58 | 8582 | 18.3 |
| 26.36 | 10548 | 46.1 |

### Example 34: Preparation and Characterization of Crystalline Pyruvic Acid Cocrystal of Compound 1

Compound 1 (300 mg) and pyruvic acid (2 eq.) were placed in methanol (3 mL). The mixture was stirred and heated up to 50° C. at 1° C./min, followed by 2 hours at 50° C. and then cooled down to 20° C. at 0.1° C./min. The mixture was then maintained under stirring at 20° C. for 1 day before evaporation under nitrogen.

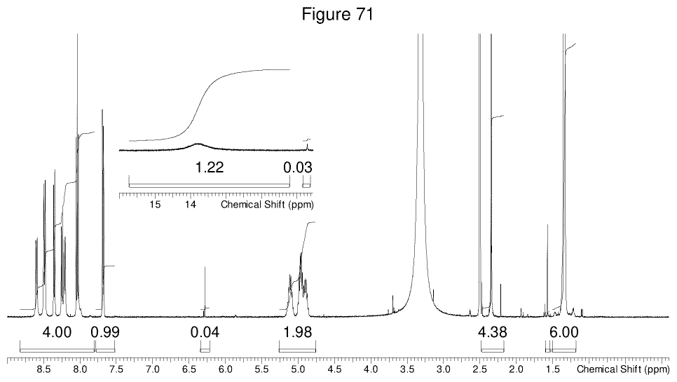

The ¹H NMR spectrum of the pyruvic acid cocrystal of Compound 1 is shown in FIG. 71.

¹H NMR (DMSO-d⁶) δ 13.78 ppm (1H, bs), 8.60 ppm/8.49 ppm/8.35 ppm/8.25 and 8.21 ppm (3H, bdₛ), 8.04 ppm (1H, t), 7.68 ppm (1H, bd), 5.20-4.82 ppm (2H, bmₛ), 2.34 ppm (3H, s), 1.34 ppm (6H, bd)

The XRPD pattern of pyruvic acid cocrystal of Compound 1 is shown in FIG. 72. The peak positions, peak heights, and relative intensities of the peaks in the XRPD pattern are listed in Table 36.

TABLE 36
XRPD Peaks of pyruvic acid
cocrystal of Compound 1

| Pos. | Intensity | Rel. Int. |
|---|---|---|
| [°2θ] | [Counts] | [%] |
| 5.08 | 9534 | 16.7 |
| 5.98 | 10036 | 29.0 |
| 6.61 | 13632 | 63.9 |
| 7.81 | 16578 | 100.0 |
| 10.24 | 5691 | 10.7 |
| 13.73 | 5083 | 14.3 |
| 14.40 | 7749 | 40.4 |
| 15.04 | 5353 | 20.1 |
| 15.30 | 4975 | 15.0 |
| 16.20 | 6512 | 31.0 |
| 16.33 | 5980 | 16.4 |
| 18.43 | 4014 | 13.3 |
| 18.99 | 6406 | 36.8 |
| 19.98 | 4419 | 18.4 |
| 20.25 | 5655 | 31.4 |
| 20.55 | 5543 | 27.3 |
| 21.62 | 3386 | 11.2 |
| 26.41 | 5595 | 39.2 |
| 27.10 | 5428 | 39.1 |

### Example 35: Amorphous Solid Dispersion (ASD) of Solid Forms of Compound 1

Solid form of Compound 1 (salt, cocrystal or free form) (10 mg) was placed in tert-butanol (10 mL) and then water (1 mL) was added for complete dissolution at room temperature. HPMCAS (40 mg) was dissolved in tert-butanol (20 mL) at room temperature. Both solutions were mixed, and the final solution was freeze dried under vacuum at 0.05 mbar and at the end, an amorphous powder was obtained.

Modulated DSC analyses were performed on the amorphous powders for determining the anhydrous glass transition temperatures (Table 37)

TABLE 37

| Compound 1 phase for ASD | |
|---|---|
| preparation with HPMCAS | Anhydrous |
| (20/80) | Tg (° C.) |
| Free Form | 89 |
| Glutaric acid cocrystal | 78 |
| Alpha-ketoglutaric acid cocrystal | 79 |
| Malonic acid cocrystal | 81 |
| Salicylic acid cocrystal | 82 |
| Maleic acid cocrystal | 83 |
| L-Malic acid cocrystal | 84 |
| Citric acid cocrystal | 85 |
| 4-Hydroxybenzoic acid cocrystal | 85 |
| Fumaric acid cocrystal | 87 |
| L-Tartaric acid cocrystal | 89 |
| Gentisic acid cocrystal | 89 |
| Oxalic acid cocrystal | 89 |
| 3-Hydroxy-2-naphtoic acid | 89 |
| cocrystal | |
| Pyroglutamic acid cocrystal | 90 |
| Glycerol cocrystal | 90 |
| Urea cocrystal | 91 |
| S-Proline cocrystal | 91 |
| Benzoic acid cocrystal | 93 |
| Glycolic acid cocrystal | 95 |
| D-Gluconic acid cocrystal | 95 |
| Caproic acid cocrystal | 97 |
| Glycine cocrystal | 98 |
| L-Serine cocrystal | 99 |
| Pyruvic acid cocrystal | 101 |
| L-Lysine cocrystal | 106 |
| Hydrochloric acid salt | 96 |
| Camphor-10-sulfonic acid salt | 99 |
| Hydrobromic acid salt | 100 |
| Benzenesulfonic acid salt | 101 |
| Ortho-phosphoric acid salt | 101 |
| Ethanesulfonic acid salt | 102 |
| p-Toluenesulfonic acid salt | 102 |
| Sulfuric acid salt | 102 |
| Naphthalene-2-sulfonic acid salt | 104 |
| Ethane-1,2-disulfonic acid salt | 118 |
| Naphthalene-1,5-disulfonic acid | 119 |
| salt | |

Developing an amorphous solid dispersion (ASD) is a formulation strategy that is usually used to manage poorly soluble drug substances, as Compound 1 is.

ASD may allow to reduce dosage or increase the load of drug substance in the drug product. It may also offer more possibilities if the pharmaceutical composition should meet specific requirements, for example if it is intended to be used by pediatric population or by patients that encounter difficulties to swallow tablets (as using ASD may allow to reduce tablet's size).

ASD may also allow to use innovative technologies as 3D printing that could be useful to obtain personalized formulations for the patients. Compared to conventional tablet presses, printed pills can be coated with a more porous surface, which helps them dissolve more quickly and without additional liquid intake.

For an amorphous solid dispersion (ASD), a high glass transition temperature (Tg) generally indicates better stability. Increasing the Tg value of an ASD may be valuable to ensure a better stability of the resulting ASD and maximize its potential uses.

The polymer selection is an important aspect of the ASD formulation. In some cases, the polymer selection can be made for other specific characteristics, like controlled release properties, resulting in the ASD having a low Tg value and consequently a potential poor stability.

The approach used in this study is to play on the solid phase of the drug through the salification or cocrystallization in order to increase the Tg value of the resulting ASD. The results highlight that the salification of Compound 1 is an interesting solution to increase the Tg value of the resulting ASD.

1. A pharmaceutically acceptable salt of a compound of formula (I) 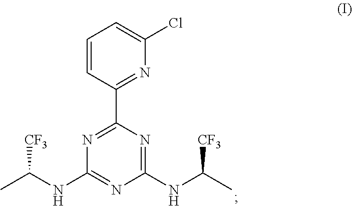 [SMILES: C[C@@H](Nc1nc(N[C@H](C)C(F)(F)F)nc(-c2cccc(Cl)n2)n1)C(F)(F)F]
wherein the pharmaceutically acceptable salt is formed using benzene sulfonic acid, (+)-camphor-10-sulfonic acid, ethane-1,2-disulfonic acid, ethanesulfonic acid, hydrobromic acid, hydrochloric acid, naphthalene-1,5-disulfonic acid, naphthalene-2-sulfonic acid, p-toluenesulfonic acid, sulfuric acid or ortho phosphoric acid.

2. A pharmaceutically acceptable salt according to claim 1, wherein the pharmaceutically acceptable salt is crystalline.

3. A pharmaceutically acceptable salt according to claim 2, wherein the pharmaceutically acceptable salt is formed using benzene sulfonic acid and the X-ray powder diffraction pattern comprises at least two peak positions, in degrees 2-theta (±0.2 degrees 2-theta), selected from the group consisting of 5.83, 8.17, 14.40 and 17.90.

4. A pharmaceutically acceptable salt according to claim 2, wherein the pharmaceutically acceptable salt is formed using (+)-camphor-10-sulfonic acid and the X-ray powder diffraction pattern comprises at least two peak positions, in degrees 2-theta (±0.2 degrees 2-theta), selected from the group consisting of 8.29, 10.19, 12.07, 18.12 and 19.97.

5. A pharmaceutically acceptable salt according to claim 2, wherein the pharmaceutically acceptable salt is formed using ethane-1,2-disulfonic acid and the X-ray powder diffraction pattern comprises at least two peak positions, in degrees 2-theta (±0.2 degrees 2-theta), selected from the group consisting of 5.59, 14.75, 18.08 and 18.87.

6. A pharmaceutically acceptable salt according to claim 5, wherein the ethane-1,2-disulfonic salt is characterized by a differential scanning calorimetry thermogram comprising an endothermic peak having an onset temperature of 304.30° C. (±2.0° C.).

7. A pharmaceutically acceptable salt according to claim 2, wherein the pharmaceutically acceptable salt is formed using ethanesulfonic acid and the X-ray powder diffraction pattern comprises at least two peak positions, in degrees 2-theta (±0.2 degrees 2-theta), selected from the group consisting of 6.86, 13.97, 14.27, 17.00 and 17.72.

8. A pharmaceutically acceptable salt according to claim 2, wherein the pharmaceutically acceptable salt is formed using hydrobromic acid and the X-ray powder diffraction pattern comprises at least two peak positions, in degrees 2-theta (±0.2 degrees 2-theta), selected from the group consisting of 5.74, 8.56, 13.63, 14.90 and 15.08.

9. A pharmaceutically acceptable salt according to claim 2, wherein the pharmaceutically acceptable salt is formed using hydrochloric acid and the X-ray powder diffraction pattern comprises at least two peak positions, in degrees 2-theta (±0.2 degrees 2-theta), selected from the group consisting of 5.73, 9.71, 13.78, 14.64 and 16.25.

10. A pharmaceutically acceptable salt according to claim 2, wherein the pharmaceutically acceptable salt is formed using naphthalene-1,5-disulfonic acid and the X-ray powder diffraction pattern comprises at least two peak positions, in degrees 2-theta (±0.2 degrees 2-theta), selected from the group consisting of 5.52, 13.79, 14.17, 19.28, 19.63 and 19.97.

11. A pharmaceutically acceptable salt according to claim 10, wherein the naphthalene-1,5-disulfonic salt is characterized by a differential scanning calorimetry thermogram comprising an endothermic peak having an onset temperature of 324.30° C. (±2.0° C.).

12. A pharmaceutically acceptable salt according to claim 2, wherein the pharmaceutically acceptable salt is formed using naphthalene-2-sulfonic acid and the X-ray powder diffraction pattern comprises at least two peak positions, in degrees 2-theta (±0.2 degrees 2-theta), selected from the group consisting of 8.41, 13.65, 26.09 and 26.47.

13. A pharmaceutically acceptable salt according to claim 2, wherein the pharmaceutically acceptable salt is formed using p-toluenesulfonic acid and the X-ray powder diffraction pattern comprises at least two peak positions, in degrees 2-theta (±0.2 degrees 2-theta), selected from the group consisting of 5.29, 8.37, 8.66, 12.96, 13.47, 20.42 and 20.64.

14. A pharmaceutically acceptable salt according to claim 2, wherein the pharmaceutically acceptable salt is formed using sulfuric acid and the X-ray powder diffraction pattern comprises at least two peak positions, in degrees 2-theta (±0.2 degrees 2-theta), selected from the group consisting of 5.80, 8.50, 13.62, 14.21, 14.87, and 18.04.

15. A pharmaceutically acceptable salt according to claim 2, wherein the pharmaceutically acceptable salt is formed using ortho phosphoric acid and the X-ray powder diffraction pattern comprises at least two peak positions, in degrees 2-theta (±0.2 degrees 2-theta), selected from the group consisting of 3.65, 5.47, 7.04, 11.01, 14.27, 14.69, 14.91 and 18.35.

16. A pharmaceutical composition comprising a pharmaceutically acceptable salt according to claim 1 and one or more pharmaceutical excipients.

17. The pharmaceutical composition of claim 16, wherein the pharmaceutical composition comprises 1-10% w/w of the compound of formula (I).

18. (canceled)

19. The pharmaceutical composition of claim 16, wherein the pharmaceutical composition comprises about 10 mg or about 40 mg of the compound of formula (I).

20. An amorphous solid dispersion prepared from a pharmaceutically acceptable salt according to claim 1 and a polymer.

21. An amorphous solid dispersion prepared from a pharmaceutically acceptable salt according to claim 20, wherein the polymer is selected from the group consisting of hydroxypropyl methyl cellulose (HPMC), hydroxypropyl methylcellulose acetate succinate (HPMCAS), hydroxypropyl methyl cellulose phthalate (HPMCP), hydroxypropyl cellulose (HPC), ethylcellulose, cellulose acetate phthalate, and polyvinylpyrrolidone (PVP), or a mixture thereof.

22. An amorphous solid dispersion prepared from a pharmaceutically acceptable salt according to claim 21, wherein the polymer is HPMCAS.

23. A pharmaceutical composition comprising a solid dispersion according to claim 20 and one or more acceptable excipients.

24-28. (canceled)

29. A method for treating a cancer characterized by the presence of an IDH1 and/or IDH2 mutation comprising administering to a patient in need thereof an amorphous solid dispersion according to claim 20.

30. The method of claim 29, wherein the cancer is characterized by the presence of an IDH1 mutation.

31. The method of claim 30, wherein the IDH1 mutation is an R132X mutation.

32. The method of claim 31, wherein the IDH1 mutation is an R132H, R132C, R132S, R132L or R132G mutation.

33. The method of claim 30 wherein the IDH1 mutation results in accumulation of R(−)-2-hydroxyglutarate in the patient.

34. The method of claim 29, wherein the cancer is characterized by the presence of an IDH2 mutation.

35. The method of claim 34, wherein the IDH2 mutation is an R140X mutation.

36. The method of claim 35, wherein the IDH2 mutation is an R140Q, R140W, or R140L mutation.

37. The method of claim 34, wherein the IDH2 mutation is an R172X mutation.

38. The method of claim 37, wherein the IDH2 mutation is an R172K or R172G mutation.

39. The method of claim 34 wherein the IDH2 mutation results in accumulation of R(−)-2-hydroxyglutarate in the patient.

40. The method of claim 29, wherein the cancer is characterized by the presence of an IDH1 mutation and an IDH2 mutation.

41. The method of claim 40, wherein the IDH1 and IDH2 mutations collectively result in accumulation of R(−)-2-hydroxyglutarate in a patient.

42. The method of claim 29, wherein the cancer is a brain tumor.

43. The pharmaceutically acceptable salt, pharmaceutical composition or amorphous solid dispersion for use according to claim 42, wherein the brain tumor is glioma.

44. The pharmaceutically acceptable salt, pharmaceutical composition or amorphous solid dispersion for use according to claim 43, wherein the glioma is a low grade glioma or a secondary high grade glioma.

45. The method of claim 43, wherein the glioma is a secondary high grade glioma, and the secondary high grade glioma is glioblastoma.

46-47. (canceled)

48. The method of claim 29, wherein the pharmaceutically acceptable salt is administered in an amount of about 10 mg, about 20 mg or about 40 mg per day, based on the amount of the compound of formula (I).

49. (canceled)

50. The method of claim 29 wherein the pharmaceutically acceptable salt is administered in an amount of about 10 mg, about 20 mg or about 40 mg, twice per day, based on the amount of the compound of formula (I).
